SEQUENCE LISTING (80 sequences)

SEQ ID NO: 1 - DNA, 1113 bp, Homo sapiens
atggcgcggcccgggctcattcattccgcgccgggcctgccagacacctgcgcccttctgcagccgcccgccgcatccgccgccgcagcccccagcatgtcgggcccagacgtcgagacgccgtccgccatccagatctgccggatcatgcggccagatgatgccaacgtggccggcaatgtccacggggggaccatcctgaagatgatcgaggaggcaggcgccatcatcagcacccggcattgcaacagccagaacggggagcgctgtgtggccgccctggctcgtgtcgagcgcaccgacttcctgtctcccatgtgcatcggtgaggtggcgcatgtcagcgcggagatcacctacacctccaagcactctgtggaggtgcaggtcaacgtgatgtccgaaaacatcctcacaggtgccaaaaagctgaccaataaggccaccctgtggtatgtgcccctgtcgctgaagaatgtggacaaggtcctcgaggtgcctcctgttgtgtattcccggcaggagcaggaggaggagggccggaagcggtatgaagcccagaagctggagcgcatggagaccaagtggaggaacggggacatcgtccagccagtcctcaacccagagccgaacactgtcagctacagccagtccagcttgatccacctggtggggccttcagactgcaccctgcacggctttgtgcacggaggtgtgaccatgaagctcatggatgaggtcgccgggatcgtggctgcacgccactgcaagaccaacatcgtcacagcttccgtggacgccattaattttcatgacaagatcagaaaaggctgcgtcatcaccatctcgggacgcatgaccttcacgagcaataagtccatggagatcgaggtgttggtggacgccgaccctgttgtggacagctctcagaagcgctaccgggccgccagtgccttcttcacctacgtgtcgctgagccaggaaggcaggtcgctgcctgtgccccagctggtgcccgagaccgaggacgagaagaagcgctttgaggaaggcaaagggcggtacctgcagatgaaggcgaagcgacagggccacgcggagcctcagccctag

SEQ ID NO: 2 - DNA, 1512 bp, Homo sapiens
atggaagccatcccatgcgtggtgggggatgaggaggtgtggacgtcggacgtgcagtaccaagtgtcgccttttaaccatggacataaggtggccaagttctgttatgcagacaagagcctgctcaacaaagccattgaggctgccctggctgcccggaaagagtgggacctgaagcctattgcagaccgggcccagatcttcctgaaggcggcagacatgctgagtgggccgcgcagggctgagatcctcgccaagaccatggtgggacagggtaagaccgtgatccaagcggagattgacgctgcagcggaactcatcgacttcttccggttcaatgccaagtatgcggtggagctggaggggcagcagcccatcagcgtgcccccgagcaccaacagcacggtgtaccggggtctggagggcttcgtggcggccatctcgccctttaacttcactgcaatcggcggcaacctggcgggggcaccggccctgatgggcaacgtggtcctatggaagcccagtgacactgccatgctggccagctatgctgtctaccgcatccttcgggaggctggcctgccccccaacatcatccagtttgtgccagctgatgggcccctatttggggacactgtcaccagctcagagcacctctgtggcatcaacttcacaggcagtgtgcccaccttcaaacacctgtggaagcaggtggcccagaacctggaccggttccacaccttcccacgcctggctggagagtgcggcggaaagaacttccacttcgtgcaccgctcggccgacgtggagagcgtggtgagcgggaccctccgctcagccttcgagtacggtggccagaagtgttccgcgtgctcgcgtctctacgtgccgcactcgctgtggccgcagatcaaagggcggctgctggaggagcacagtcggatcaaagtgggcgaccctgcagaggattttgggaccttcttctctgcagtgattgatgccaagtcctttgcccgtatcaagaagtggctggagcacgcacgctcctcacccagcctcaccatcctggccgggggcaagtgtgatgactccgtgggctactttgtggagccctgcatcgtggagagcaaggaccctcaggagcccatcatgaaggaggagatcttcgggcctgtactgtctgtgtacgtctacccggatgacaagtacaaggagacgctgcagctggttgacagcaccaccagctatggcctcacgggggcagtgttctcccaggataaggacgtcgtgcaggaggccacaaaggtgctgaggaatgctgccggcaacttctacatcaacgacaagtccactggctcgatagtgggccagcagccctttgggggggcccgagcctctggaaccaatgacaagccagggggcccacactacatcctgcgctggacgtcgccgcaggtcatcaaggagacacataagcccctgggggactggagctacgcgtacatgcagtga

SEQ ID NO: 3 - DNA, 912 bp, Homo sapiens
atggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccaggattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcgatgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattgaagttaagagctga

SEQ ID NO: 4 - DNA, 1056 bp, Homo sapiens
atgtcgttcttcccggagctttactttaacgtggacaatggctacttggagggactggtgcgcggcctgaaggccggggtgctcagccaggccgactacctcaacctggtgcagtgcgagacgctagaggacttgaaactgcatctgcagagcactgattatggtaacttcctggccaacgaggcatcacctctgacggtgtcagtcatcgatgaccggctcaaggagaagatggtggtggagttccgccacatgaggaaccatgcctatgagccactcgccagcttcctagacttcattacttacagttacatgatcgacaacgtgatcctgctcatcacaggcacgctgcaccagcgctccatcgctgagctcgtgcccaagtgccacccactaggcagcttcgagcagatggaggccgtgaacattgctcagacacctgctgagctctacaatgccattctggtggacacgcctcttgcggcttttttccaggactgcatttcagagcaggaccttgacgagatgaacatcgagatcatccgcaacaccctctacaaggcctacctggagtccttctacaagttctgcaccctactgggcgggactacggctgatgccatgtgccccatcctggagtttgaagcagaccgccgcgccttcatcatcaccatcaattctttcggcacagagctgtccaaagaggaccgtgccaagctctttccacactgtgggcggctctaccctgagggcctggcgcagctggctcgggctgacgactatgaacaggtcaagaacgtggccgattactacccggagtacaagctgctcttcgagggtgcaggtagcaaccctggagacaagacgctggaggaccgattctttgagcacgaggtaaagctgaacaagttggccttcctgaaccagttccactttggtgtcttctatgccttcgtgaagctcaaggagcaggagtgtcgcaacatcgtgtggatcgctgaatgtatcgcccagcgccaccgcgccaaaatcgacaactacatccctatcttctag

SEQ ID NO: 5 - DNA, 396 bp, Homo sapiens
atggatgtcctctttgtagccatctttgctgtgccacttatcctgggacaagaatatgaggatgaagaaagactgggagaggatgaatattatcaggtggtctattattatacagtcacccccagttatgatgactttagtgcagatttcaccattgattactccatatttgagtcagaggacaggctgaacaggttggataaggacataacagaagcaatagagactaccattagtcttgaaacagcacgtgcagaccatccgaagcctgtaactgtgaaaccagtaacaacggaacctagtccagatctgaacgatgccgtgtccagtttgcgaagtcctattcccctcctcctgtcgtgtgcctttgttcaggtggggatgtatttcatgtag

SEQ ID NO: 6 - DNA, 483 bp, Homo sapiens
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagagtcactggaactggaggacccgtcttctgggctgggtgtga

SEQ ID NO: 7 - DNA, 810 bp, Homo sapiens
atgagcgccgcgctcttcagcctggacggcccggcgcgcggcgcgccctggcctgcggagcctgcgcccttctacgaaccgggccgggcgggcaagccgggccgcggggccgagccaggggccctaggcgagccaggcgccgccgcccccgccatgtacgacgacgagagcgccatcgacttcagcgcctacatcgactccatggccgccgtgcccaccctggagctgtgccacgacgagctcttcgccgacctcttcaacagcaatcacaaggcgggcggcgcggggcccctggagcttcttcccggcggccccgcgcgccccttgggcccgggccctgccgctccccgcctgctcaagcgcgagcccgactggggcgacggcgacgcgcccggctcgctgttgcccgcgcaggtggccgcgtgcgcacagaccgtggtgagcttggcggccgcagggcagcccaccccgcccacgtcgccggagccgccgcgcagcagccccaggcagacccccgcgcccggccccgcccgggagaagagcgccggcaagaggggcccggaccgcggcagccccgagtaccggcagcggcgcgagcgcaacaacatcgccgtgcgcaagagccgcgacaaggccaagcggcgcaaccaggagatgcagcagaagttggtggagctgtcggctgagaacgagaagctgcaccagcgcgtggagcagctcacgcgggacctggccggcctccggcagttcttcaagcagctgcccagcccgcccttcctgccggccgccgggacagcagactgccggtaa

SEQ ID NO: 8 - DNA, 1443 bp, Homo sapiens
atgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccatccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataa

SEQ ID NO: 9 - DNA, 519 bp, Homo sapiens
atggcatcagatgaaggcaaactttttgttggagggctgagttttgacaccaatgagcagtcgctggagcaggtcttctcaaagtacggacagatctctgaagtggtggttgtgaaagacagggagacccagagatctcggggatttgggtttgtcacctttgagaacattgacgacgctaaggatgccatgatggccatgaatgggaagtctgtagatggacggcagatccgagtagaccaggcaggcaagtcgtcagacaaccgatcccgtgggtaccgtggtggctctgccgggggccggggcttcttccgtgggggccgaggacggggccgtgggttctctagaggaggaggggaccgaggctatggggggaaccggttcgagtccaggagtgggggctacggaggctccagagactactatagcagccggagtcagagtggtggctacagtgaccggagctcgggcgggtcctacagagacagttatgacagttacgctacacacaacgagtaa

SEQ ID NO: 10 - DNA, 528 bp, Homo sapiens
atgtcgcacggagccgggctcgtccgcaccacgtgcagcagcggcagcgcgctcggacccggggccggcgcggcccagcccagcgcgagccccttggaggggctgctggacctcagctacccccgcacccacgcggccctgctgaaagtggcgcaaatggtcaccctgctgattgccttcatctgtgtgcggagctccctgtggaccaactacagcgcctacagctactttgaagtggtcaccatttgcgacttgataatgatcctcgccttttacctggtccacctcttccgcttctaccgcgtgctcacctgtatcagctggcccctgtcggaacttctgcactatttaatcggtaccctgctcctcctcatcgcctccattgtggcagcttccaagagttacaaccagagcggactggtagccggagcgatctttggtttcatggccaccttcctctgcatggcaagcatatggctgtcctataagatctcgtgtgtaacccagtccacagatgcagccgtctga

SEQ ID NO: 11 - DNA, 1614 bp, Homo sapiens
atggctgcccagtgtgtcacaaaggtggcgctgaatgtttcctgtgccaatcttttggataaagatatagggtcaaagtcagaccctttatgtgtgttgtttttgaatacaagtggtcaacagtggtatgaggttgagcgcacagaaaggattaagaattgcttgaatccccaattttccaagacatttattattgattactactttgaagtggttcagaaattgaaatttggggtttatgacatcgacaacaaaactattgagctgagtgatgatgacttcttaggggaatgtgaatgtacccttggacaaattgtttccagcaagaagctaactcgaccactggtgatgaaaactggcagacctgcaggaaaagggagcattacgatttcagctgaagaaataaaagataatagagtggtcttgtttgaaatggaagccagaaaactggataataaggatctatttggaaagtcagacccatacctggaattccacaagcagacatctgatggaaactggctaatggttcatcggacagaggttgttaaaaacaacttgaatcctgtttggaggcctttcaagatctctcttaactcactgtgttacggagatatggacaaaaccattaaggtggagtgttatgattatgacaatgatgggtcacatgatctcattggaacatttcagaccaccatgacaaaactgaaagaagcctccagaagctcacctgttgaatttgaatgcataaatgagaaaaaaaggcaaaagaaaaaaagctacaagaattcaggtgttatcagtgtgaaacagtgtgagattacagtagaatgcacattccttgactatataatgggaggatgtcagctgaattttactgtgggagtggacttcactggctccaatggtgacccaaggtctccagactcccttcattacatcagccccaatggcgttaatgagtatttgactgctctctggtctgtgggactggtcattcaagattatgatgctgataagatgtttccagcttttggttttggcgctcagatacctcctcagtggcaggtatcacatgaatttccaatgaacttcaacccatccaatccctactgcaatggaatccaaggcattgtagaggcgtatcggtcttgtcttcctcagataaaactctatggaccaactaatttttctccaatcataaatcacgtggccaggtttgctgctgcagccacgcaacagcagacagcttctcaatattttgtgcttttgattattactgatggtgtgatcacagaccttgatgaaaccagacaagctatagttaatgcctccaggctgcctatgtccatcataattgttggagttggaggtgctgacttcagcgccatggagtttctggatggtgatggtggaagtctccgctccccattgggcgaagtggccatcagagatattgtccagtttgtgcctttcagacagttccagaatgctccaaaagaagcacttgctcagtgtgtcttggcagagattccccagcaggtggtgggctacttcaatacatacaaactccttcctcccaagaacccagccacgaaacaacagaagcagtga

SEQ ID NO: 12 - DNA, 933 bp, Homo sapiens
atggtctacaagactctcttcgctctttgcatcttaactgcaggatggagggtacagagtctgcctacatcagctcctttgtctgtttctcttccgacaaacattgtaccaccgaccaccatctggactagctctccacaaaacactgatgcagacactgcctccccatccaacggcactcacaacaactcggtgctcccagttacagcatcagccccaacatctctgcttcctaagaacatttccatagagtccagagaagaggagatcaccagcccaggttcgaattgggaaggcacaaacacagacccctcaccttctgggttctcgtcaacaagcggtggagtccacttaacaaccacgttggaggaacacagctcgggcactcctgaagcaggcgtggcagctacactgtcgcagtccgctgctgagcctcccacactcatctcccctcaagctccagcctcatcaccctcatccctatcaacctcaccacctgaggtcttttctgcctccgttactaccaaccatagctccactgtgaccagcacccaacccactggagctccaactgcaccagagtccccgacagaggagtccagctctgaccacacacccacttcacatgccacagctgagccagtaccccaggagaaaacacccccaacaactgtgtcaggcaaagtgatgtgtgagctcatagacatggagaccaccaccacctttcccagggtgatcatgcaggaagtagaacatgcattaagttcaggcagcatcgccgccattaccgtgacagtcattgccgtggtgctgctggtgtttggagttgcagcctacctaaaaatcaggcattcctcctatggaagacttttggacgaccatgactacgggtcctggggaaactacaacaaccctctgtacgatgactcctaa

SEQ ID NO: 13 - DNA, 492 bp, Homo sapiens
atgtcactcctcttgctggtggtctcagcccttcacatcctcattcttatactgcttttcgtggccactttggacaagtcctggtggactctccctgggaaagagtccctgaatctctggtacgactgcacgtggaacaacgacaccaaaacatgggcctgcagtaatgtcagcgagaatggctggctgaaggcggtgcaggtcctcatggtgctctccctcattctctgctgtctctccttcatcctgttcatgttccagctctacaccatgcgacgaggaggtctcttctatgccaccggcctctgccagctttgcaccagcgtggcggtgtttactggcgccttgatctatgccattcacgccgaggagatcctggagaagcacccgcgagggggcagcttcggatactgcttcgccctggcctgggtggccttccccctcgccctggtcagcggcatcatctacatccacctacggaagcgggagtga

SEQ ID NO: 14 - DNA, 684 bp, Homo sapiens
atgagggtagcaggtgctgcaaagttggtggtagctgtggcagtgtttttactgacattttatgttatttctcaagtatttgaaataaaaatggatgcaagtttaggaaatctatttgcaagatcagcattggacacagctgcacgttctacaaagcctcccagatataagtgtgggatctcaaaagcttgccctgagaagcattttgcttttaaaatggcaagtggagcagccaacgtggtgggacccaaaatctgcctggaagataatgttttaatgagtggtgttaagaataatgttggaagagggatcaatgttgccttggcaaatggaaaaacaggagaagtattagacactaaatattttgacatgtggggaggagatgtggcaccatttattgagtttctgaaggccatacaagatggaacaatagttttaatgggaacatacgatgatggagcaaccaaactcaatgatgaggcacggcggctcattgctgatttggggagcacatctattactaatcttggttttagagacaactgggtcttctgtggtgggaagggcattaagacaaaaagcccttttgaacagcacataaagaacaataaggatacaaacaaatatgaaggatggcctgaagttgtagaaatggaaggatgcatcccccagaagcaagactaa

SEQ ID NO: 15 - DNA, 1329 bp, Homo sapiens
atggcggagggtgaagggtacttcgccatgtctgaggacgagctggcctgcagcccctacatccccctaggcggcgacttcggcggcggcgacttcggcggcggcgacttcggcggcggcgacttcggcggtggcggcagcttcggtgggcattgcttggactattgcgaaagccctacggcgcactgcaatgtgctgaactgggagcaagtgcagcggctggacggcatcctgagcgagaccattccgattcacgggcgcggcaacttccccacgctcgagctgcagccgagcctgatcgtgaaggtggtgcggcggcgcctggccgagaagcgcattggcgtccgcgacgtgcgcctcaacggctcggcagccagccatgtcctgcaccaggacagcggcctgggctacaaggacctggacctcatcttctgcgccgacctgcgcggggaaggggagtttcagactgtgaaggacgtcgtgctggactgcctgttggacttcttacccgagggggtgaacaaagagaagatcacaccactcacgctcaaggaagcttatgtgcagaaaatggttaaagtgtgcaatgactctgaccgatggagtcttatatccctgtcaaacaacagtggcaaaaatgtggaactgaaatttgtggattccctccggaggcagtttgaattcagtgtagattcttttcaaatcaaattagactctcttctgctcttttatgaatgttcagagaacccaatgactgagacatttcaccccacaataatcggggagagcgtctatggcgatttccaggaagcctttgatcacctttgtaacaagatcattgccaccaggaacccagaggaaatccgagggggaggcctgcttaagtactgcaacctcttggtgaggggctttaggcccgcctctgatgaaatcaagacccttcaaaggtatatgtgttccaggtttttcatcgacttctcagacattggagagcagcagagaaaactggagtcctatttgcagaaccactttgtgggattggaagaccgcaagtatgagtatctcatgacccttcatggagtggtaaatgagagcacagtgtgcctgatgggacatgaaagaagacagactttaaaccttatcaccatgctggctatccgggtgttagctgaccaaaatgtcattcctaatgtggctaatgtcacttgctattaccagccagccccctatgtagcagatgccaactttagcaattactacattgcacaggttcagccagtattcacgtgccagcaacagacctactccacttggctaccctgcaattaa

SEQ ID NO: 16 - DNA, 393 bp, Homo sapiens
atgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtag

SEQ ID NO: 17 - DNA, 1146 bp, Homo sapiens
atgtccggggtggtgcggaccctcagccgctgcctgctgccggccgaggccggcggggcccgcgagcgcagggcgggcagcggcgcgcgcgacgcggagcgcgaggcccggaggcgtagccgcgacatcgacgcgctgctggcccgcgagcggcgcgcggtccggcgcctggtgaagatcctgctgctgggcgcgggcgagagcggcaagtccacgttcctcaagcagatgcgcatcatccacggccgcgagttcgaccagaaggcgctgctggagttccgcgacaccatcttcgacaacatcctcaagggctcaagggttcttgttgatgcacgagataagcttggcattccttggcagtattctgaaaatgagaagcatgggatgttcctgatggccttcgagaacaaggcggggctgcctgtggagccggccaccttccagctgtacgtcccggccctgagcgcactctggagggattctggcatcagggaggctttcagccggagaagcgagtttcagctgggggagtcggtgaagtacttcctggacaacttggaccggatcggccagctgaattactttcctagtaagcaagatatcctgctggctaggaaagccaccaagggaattgtggagcatgacttcgttattaagaagatcccctttaagatggtggatgtgggcggccagcggtcccagcgccagaagtggttccagtgcttcgacgggatcacgtccatcctgttcatggtctcctccagcgagtacgaccaggtcctcatggaggacaggcgcaccaaccggctggtggagtccatgaacatcttcgagaccatcgtcaacaacaagctcttcttcaacgtctccatcattctcttcctcaacaagatggacctcctggtggagaaggtgaagaccgtgagcatcaagaagcacttcccggacttcaggggcgacccgcacaggctggaggacgtccagcgctacctggtccagtgcttcgacaggaagagacggaaccgcagcaagccactcttccaccacttcaccaccgccatcgacaccgagaacgtccgcttcgtgttccatgctgtgaaagacaccatcctgcaggagaacctgaaggacatcatgctgcagtga

SEQ ID NO: 18 - DNA, 618 bp, Homo sapiens
atgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaa

SEQ ID NO: 19 - DNA, 360 bp, Homo sapiens
atgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcactccggaacttgtcatctccaacgacaaaaggagcttttgccactga

SEQ ID NO: 20 - DNA, 399 bp, Homo sapiens
atgaaccacattgtgcaaaccttctctcctgtcaacagcggccagcctcccaactacgagatgctcaaggaggagcaggaagtggctatgctgggggtgccccacaaccctgctcccccgatgtccaccgtgatccacatccgcagcgagacctccgtgcctgaccatgtggtctggtccctgttcaacaccctcttcatgaacacctgctgcctgggcttcatagcattcgcgtactccgtgaagtctagggacaggaagatggttggcgacgtgaccggggcccaggcctatgcctccaccgccaagtgcctgaacatctgggccctgattttgggcatcttcatgaccattctgctcatcatcatcccagtgttggtcgtccaggcccagcgatag

SEQ ID NO: 21 - DNA, 8133 bp, Homo sapiens
atgtctgacaaaatgtctagcttcctacatattggagacatttgttctctgtacgcggagggatcgacaaatggatttattagcaccttgggcctggttgatgatcgttgtgttgtacagccagaaaccggggaccttaacaatccacctaagaaattcagagactgcctctttaagctatgtcccatgaaccgctactctgcccaaaagcagttctggaaagccgctaagcctggggccaacagcaccacagacgcagtgctactcaacaaactgcaccacgctgcagacttggaaaagaagcagaatgagacagaaaacaggaaattgctggggaccgtaatccagtatggcaatgtgatccagctcctgcatttgaaaagtaataaatacctaacagtgaataagaggcttcctgctctgttggagaagaatgccatgagagtcacattggacgaggctggaaatgaagggtcctggttttatattcagccattctacaagctgcgatccattggagacagcgtggtcataggtgacaaggtggttctgaaccccgtcaatgctggtcagcccctacatgctagcagccatcaactggtagataacccaggctgcaatgaggtcaattccgtcaactgcaatacaagctggaaaatagtccttttcatgaaatggagtgataacaaagacgacatattaaaggggggtgacgtggtgaggctgtttcatgctgagcaggagaagtttctcacctgtgacgaacacaggaagaagcagcacgtcttcctgagaaccacgggccggcagtcggccacatctgccaccagttcaaaagccctgtgggaggtggaggtggtccagcatgacccatgtcggggcggagcagggtattggaacagccttttccgtttcaagcatctggccacggggcattacttggcagcagaggtagaccctgactttgaggaagaatgcctggagtttcagccctcagtggaccctgatcaggacgcctctcgaagtaggttgcggaatgcccaagaaaagatggtatactccctggtctctgtgcctgaaggcaatgacatctcctccattttcgagctagatcccaccactctgcgtggaggtgacagccttgtcccaaggaactcttatgttcggctcagacacctatgtactaatacctgggttcacagcacaaatattcctattgacaaggaagaagaaaagcccgtgatgctgaaaattggcacctctcctgtgaaggaggataaggaagcatttgccatagttccggtttctcctgctgaagttcgggacctggactttgccaatgatgccagcaaggtgctgggctccattgctgggaagctagagaagggcaccatcacccagaatgaaaggaggtctgtaaccaagctgctagaagatttggtttacttcgtcactggtggaactaattctggtcaagatgttctcgaagttgtcttctccaagcccaacagagaacggcagaaactgatgagagaacagaatattctcaagcagatcttcaagttgttacaagccccattcacagactgcggtgatggcccaatgcttcggctggaagagctcggggaccagcggcacgctcctttcagacacatctgccggctctgctacagggtgctgagacactcgcagcaagactacaggaagaaccaggagtatatagccaagcagtttggcttcatgcagaagcagattggctatgatgtgttggctgaagacactatcactgccctgctccacaataatcggaaactcctggaaaaacacattaccgcggcagagattgacacatttgtcagcctggtgcgaaagaacagggagcccagattcttagattacctctccgacctctgtgtctccatgaacaaatcaattccagtgacccaggaactgatatgtaaagctgtgctgaaccccaccaacgctgacatcctgattgagaccaagttggttctttctcgttttgaatttgaaggtgtctcttccactggagagaatgctctggaggcaggagaagacgaggaagaggtgtggctgttttggagggacagcaacaaagagattcgcagcaagagtgtgagggaattggctcaggatgctaaagaagggcagaaggaggaccgagacgttctcagctactacagatatcagctgaacctctttgcgaggatgtgtctggaccgccaatacctggccatcaacgaaatctcaggccagctggatgtcgatctcattctccgctgcatgtctgacgagaacctgccctatgacctcagggcgtccttctgccgcctcatgcttcacatgcatgtggaccgagatccccaggaacaagtcacccccgtgaaatatgcccgcctctggtcggagattccctcggagatcgccattgacgactatgatagtagtggagcttccaaagatgaaattaaggagagatttgctcagaccatggagtttgtggaggagtatttaagagatgtggtttgtcagaggttccctttctctgataaagagaagaataagcttacgtttgaggttgtaaatttagctaggaatctcatatactttggtttctacaacttctctgaccttctacgattaactaagatccttctggccatattggactgtgtacatgtgacaacaatcttccccattagcaagatggcgaaaggagaagagaataaaggcagtaacgtgatgagatctattcatggcgtgggagagctgatgacccaggtggtgctccggggaggaggctttttgcccatgactcccatggctgctgcccctgaaggcaatgtgaagcaggcagagcctgagaaggaggacatcatggtcatggacaccaagctgaagatcattgagatactccagtttattttgaatgtgaggttggattataggatctcctgcctcctgtgtatatttaagcgagagtttgatgaaagcaattcccagacttcagaaacatcctccggaaacagcagccaagaagggccaagtaatgtaccaggtgctcttgactttgaacacattgaagaacaagcagaaggcatctttggaggaagtgaggagaacaccccactggacttggatgaccacggcggcagaacctttctccgtgtcctgctccacttgacgatgcatgactacccacccctggtgtcaggggccctgcagctcctcttccggcacttcagccagaggcaggaggtgctccaggccttcaaacaggttcaactgctggttaccagccaagatgtggacaactacaaacagatcaaacaagacttggatcaactgaggtccatcgtggaaaagtcagagctttgggtgtacaaagggcagggccccgatgagactatggatggtgcatctggagaaaatgaacataagaaaacggaggagggaaataacaagccacaaaagcatgaaagcaccagcagctacaactacagagtggtcaaagagattttgattcggcttagcaaactctgtgttcaagagagtgcctcagtgagaaagagcaggaagcagcaacagcgtctgctccggaacatgggcgcgcacgccgtggtgctggagctgctgcagattccctatgagaaggccgaagataccaagatgcaagagataatgaggttggctcatgaatttttgcagaatttctgcgcaggcaaccagcagaatcaagctttgctacataaacacataaacctgtttctcaacccagggatcctggaggcagtaaccatgcagcacatcttcatgaacaatttccagctttgcagtgagatcaacgagagagttgttcagcacttcgttcactgcatagagactcacggtcggaatgtccagtatataaagttcttacagacaattgtcaaggcagaagggaaatttattaaaaaatgccaagacatggttatggccgagctggtcaattcgggagaggatgtcctcgtgttctacaacgacagagcctctttccagactctgatccagatgatgcggtcagaacgggatcggatggatgagaacagccctctcatgtaccacatccacttggtcgagctcctggctgtgtgcacggagggtaagaatgtctacacagagatcaagtgcaactccctgctcccgctggatgacatcgttcgcgtggtgacccacgaggactgcatccctgaggttaaaattgcatacattaacttcctgaatcactgctatgtggatacagaggtggaaatgaaggagatttataccagcaatcacatgtggaaattgtttgagaatttccttgtagacatctgcagggcctgtaacaacactagtgacaggaaacatgcagactcgattttggagaagtatgtcaccgaaatcgtcatgagtattgttactactttcttcagctctcccttctcagaccagagtacgactttgcagactcgccagcctgtctttgtgcaactgctgcaaggcgtgttcagggtttaccactgcaactggttaatgccaagccaaaaagcctccgtggagagctgtattcgggtgctgtctgatgtagccaagagccgggccattgccattcccgtggacctggacagccaagtcaacaacctctttctcaagtcccacagcattgtgcagaaaacagccatgaactggcggctctcagcccgcaatgccgcacgcagggactctgttctggcagcttccagagactaccggaatatcattgagagattgcaggacatcgtctccgcgctggaggaccgtctcaggcccctggtgcaggcagagttatctgtgctcgtggatgttctccacagacccgagctgcttttcccagagaacacagacgccagaaggaaatgtgaaagtggcggtttcatttgcaagttaataaagcatacaaaacagctgctagaagaaaatgaagagaagctctgcattaaggtcctacagaccctgagggaaatgatgaccaaagatagaggctatggagaaaagggtgaggcgctcaggcaagttctggtcaaccgttactatggaaacgtcagaccttcgggacgaagagagagccttaccagctttggcaatggcccactgtcagcaggaggacccggcaagcccgggggaggagggggaggttccggatccagctctatgagcaggggtgagatgagtctggccgaggttcagtgtcaccttgacaaggagggggcttccaatctagttatcgacctcatcatgaacgcatccagtgaccgagtgttccatgaaagcattctcctggccattgcccttctggaaggaggcaacaccaccatccagcactcctttttctgtcgcttgacagaagataagaagtcagagaaattctttaaggtgttttatgaccggatgaaggtggcccagcaagaaatcaaagcaacagtgacagtgaacaccagtgacttgggaaataaaaagaaagacgatgaggtagacagggatgccccatcacggaaaaaagctaaagagcccacaacacagataacagaagaggtccgggatcagctcctggaggcctccgctgccaccaggaaagccttcaccactttcaggagggaggctgatcccgacgaccactaccagcctggagagggcacccaggccactgccgacaaggccaaggacgacctggagatgagcgcggtcatcaccatcatgcagcccatcctccgcttccttcagctcctgtgtgaaaaccacaaccgagacctgcagaacttcctccgttgccaaaataacaagaccaactacaatttggtatgtgagaccctgcagtttctggactgtatttgtggaagcacaactggaggccttggtcttctgggcttgtatataaatgaaaagaacgtagcgcttatcaaccaaaccctggaaagtctgaccgaatactgtcaaggaccttgccatgagaaccagaactgcatagccacccatgaatccaatggcattgacatcatcacagccctgatcctcaatgatatcaatcctttgggaaagaagaggatggaccttgtgttagaactgaagaacaatgcctcgaagttgctcctggccatcatggaaagcaggcacgacagtgaaaacgcagagaggatactttataacatgaggcccaaggaactggtggaagtgatcaagaaagcctacatgcaaggtgaagtggaatttgaggatggagaaaacggtgaggatggggcggcgtcccccaggaacgtggggcacaacatctacatattagcccatcagttggctcggcataacaaagaacttcagagcatgctgaaacctggtggccaagtggacggagatgaagccctggagttttatgccaagcacacggcgcagatagagattgtcagattagaccgaacaatggaacagatagtctttcccgtgcccagcatatgtgaattcctaaccaaggagtcaaaactacgaatttactatactacagagagagacgaacaaggcagcaaaatcaatgatttctttctgcggtctgaagacctcttcaatgaaatgaattggcagaagaaactgagagcccagcccgtgttgtactggtgtgcccgcaacatgtctttctggagcagcatttcgtttaacctggccgtcctgatgaacctgctggtggcgtttttctacccgtttaagggagtccgaggaggaaccctggagccccactggtcgggactcctgtggacagccatgctcatctctctggccatcgtcattgccctccccaagccccatggcatccgggccttaattgcctccacaattctacgactgatattttcagtcgggttacaacccacgttgtttcttctgggcgctttcaatgtatgcaataaaatcatctttctaatgagctttgtgggcaactgtgggacattcacaagaggctaccgagccatggttctggatgttgagttcctctatcatttgttgtatctggtgatctgtgccatggggctctttgtccatgaattcttctacagtctgctgctttttgatttagtgtacagagaagagactttgcttaatgtcattaaaagtgtcactcgcaatggacggtccatcatcctgacagcagttctggctctgatcctcgtttacctgttctcaatagtgggctatcttttcttcaaggatgactttatcttggaagtagataggctgcccaatgaaacagctgttccagaaaccggcgagagtttggcaagcgagttcctgttctccgatgtgtgtagggtggagagtggggagaactgctcctctcctgcacccagagaagagctggtccctgcagaagagacggaacaggataaagagcacacatgtgagacgctgctgatgtgcattgtcactgtgctgagtcacgggctgcggagcgggggtggagtaggagatgtactcaggaagccgtccaaagaggaacccctgtttgctgctagagttatttatgacctcttgttcttcttcatggtcatcatcattgttcttaacctgatttttggggttatcattgacacttttgctgacctgaggagtgagaagcagaagaaggaagagatcttgaagaccacgtgctttatctgtggcttggaaagagacaagtttgacaacaagactgtcacctttgaagagcacatcaaggaagaacacaacatgtggcactatctgtgcttcatcgtcctggtgaaagtaaaggactccaccgaatatactgggcctgagagttacgtggcagaaatgatcaaggaaagaaaccttgactggttccccaggatgagagccatgtcattggtcagcagtgattctgaaggagaacagaatgagctgagaaacctgcaggagaagctggagtccaccatgaaacttgtcacgaacctttctggccagctgtcggaattaaaggatcagatgacagaacaaaggaagcagaaacaaagaattggtcttctaggacatcctcctcacatgaatgtcaacccacaacaaccagcataa

SEQ ID NO: 22 - DNA, 996 bp, Homo sapiens
atgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttga

SEQ ID NO: 23 - DNA, 393 bp, Homo sapiens
atgttaccatttctgtttttttccaccctgttttcttccatatttactgaagctcagaagcagtattgggtctgcaactcatccgatgcaagtatttcatacacctactgtgggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattag

SEQ ID NO: 24 - DNA, 1182 bp, Homo sapiens
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 25 - DNA, 594 bp, Homo sapiens
atgtttggcctcaaaagaaacgcggtaatcggactcaacctctactgtgggggggccggcttgggggccggcagcggcggcgccacccgcccgggagggcgacttttggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatag

SEQ ID NO: 26 - DNA, 186 bp, Homo sapiens
atggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctga

SEQ ID NO: 27 - DNA, 1623 bp, Homo sapiens
atggctcgtcatttgcgcgaataccaggacctcctcaacgtcaagatggctctggatatagaaatcgctgcgtacagaaaactcctggagggtgaagagactagatttagcacatttgcaggaagcatcactgggccactgtatacacaccgacccccaatcacaatatccagtaagattcagaaacccaaggtggaagctcccaagcttaaggtccaacacaaatttgtcgaggagatcatagaggaaaccaaagtggaggatgagaagtcagaaatggaagaggccctgacagccattacagaggaattggccgtttccatgaaggaagagaagaaagaagcagcagaagaaaaggaagaggaacccgaagctgaagaagaagaagtagctgccaaaaagtctccagtgaaagcaactgcacctgaagttaaagaagaggaaggggaaaaggaggaagaagaaggccaggaagaagaggaggaagaagatgagggagctaagtcagaccaagccgaagagggaggatccgagaaggaaggctctagtgaaaaagaggaaggtgagcaggaagaaggagaaacagaagctgaagctgaaggagaggaagccgaagctaaagaggaaaagaaagtggaggaaaagagtgaggaagtggctaccaaggaggagctggtggcagatgccaaggtggaaaagccagaaaaagccaagtctcctgtgccaaaatcaccagtggaagagaaaggcaagtctcctgtgcccaagtcaccagtggaagagaaaggcaagtctcctgtgcccaagtcaccagtggaagagaaaggcaagtctcctgtgccgaaatcaccagtggaagagaaaggcaagtctcctgtgtcaaaatcaccagtggaagagaaagccaaatctcctgtgccaaaatcaccagtggaagaggcaaagtcaaaagcagaagtggggaaaggtgaacagaaagaggaagaagaaaaggaagtcaaggaagctcccaaggaagagaaggtagagaaaaaggaagagaaaccaaaggatgtgccagagaagaagaaagctgagtcccctgtaaaggaggaagctgtggcagaggtggtcaccatcaccaaatcggtaaaggtgcacttggagaaagagaccaaagaagaggggaagccactgcagcaggagaaagagaaggagaaagcgggaggagagggaggaagtgaggaggaagggagtgataaaggtgccaagggatccaggaaggaagacatagctgtcaatggggaggtagaaggaaaagaggaggtagagcaggagaccaaggaaaaaggcagtgggagggaagaggagaaaggcgttgtcaccaatggcctagacttgagcccagcagatgaaaagaaggggggtgataaaagtgaggagaaagtggtggtgaccaaaacggtagaaaaaatcaccagtgaggggggagatggtgctaccaaatacatcactaaatctgtaaccgtcactcaaaaggttgaagagcatgaagagacctttgaggagaaactagtgtctactaaaaaggtagaaaaagtcacttcacacgccatagtaaaggaagtcacccagagtgactaa

SEQ ID NO: 28 - DNA, 825 bp, Homo sapiens
atggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatga

SEQ ID NO: 29 - DNA, 1605 bp, Homo sapiens
atgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatga

SEQ ID NO: 30 - DNA, 2214 bp, Homo sapiens
atggggggatgcacggtgaagcctcagctgctgctcctggcgctcgtcctccacccctggaatccctgtctgggtgcggactcggagaagccctcgagcatccccacagataaattattagtcataactgtagcaacaaaagaaagtgatggattccatcgatttatgcagtcagccaaatatttcaattatactgtgaaggtccttggtcaaggagaagaatggagaggtggtgatggaattaatagtattggagggggccagaaagtgagattaatgaaagaagtcatggaacactatgctgatcaagatgatctggttgtcatgtttactgaatgctttgatgtcatatttgctggtggtccagaagaagttctaaaaaaattccaaaaggcaaaccacaaagtggtctttgcagcagatggaattttgtggccagataaaagactagcagacaagtatcctgttgtgcacattgggaaacgctatctgaattcaggaggatttattggctatgctccatatgtcaaccgtatagttcaacaatggaatctccaggataatgatgatgatcagctcttttacactaaagtttacattgatccactgaaaagggaagctattaacatcacattggatcacaaatgcaaaattttccagaccttaaatggagctgtagatgaagttgttttaaaatttgaaaatggcaaagccagagctaagaatacattttatgaaacattaccagtggcaattaatggaaatggacccaccaagattctcctgaattattttggaaactatgtacccaattcatggacacaggataatggctgcactctttgtgaattcgatacagtcgacttgtctgcagtagatgtccatccaaacgtatcaataggtgtttttattgagcaaccaaccccttttctacctcggtttctggacatattgttgacactggattacccaaaagaagcacttaaactttttattcataacaaagaagtttatcatgaaaaggacatcaaggtattttttgataaagctaagcatgaaatcaaaactataaaaatagtaggaccagaagaaaatctaagtcaagcggaagccagaaacatgggaatggacttttgccgtcaggatgaaaagtgtgattattactttagtgtggatgcagatgttgttttgacaaatccaaggactttaaaaattttgattgaacaaaacagaaagatcattgctcctcttgtaactcgtcatggaaagctgtggtccaatttctggggagcattgagtcctgatggatactatgcacgatctgaagattatgtggatattgttcaagggaatagagtaggagtatggaatgtcccatatatggctaatgtgtacttaattaaaggaaagacactccgatcagagatgaatgaaaggaactattttgttcgtgataaactggatcctgatatggctctttgccgaaatgctagagaaatgggtgtatttatgtacatttctaatagacatgaatttggaaggctattatccactgctaattacaatacttcccattataacaatgacctctggcagatttttgaaaatcctgtggactggaaggaaaagtatataaaccgtgattattcaaagattttcactgaaaatatagttgaacagccctgtccagatgtcttttggttccccatattttctgaaaaagcctgtgatgaattggtagaagaaatggaacattacggcaaatggtctgggggaaaacatcatgatagccgtatatctggtggttatgaaaatgtcccaactgatgatatccacatgaagcaagttgatctggagaatgtatggcttcattttatccgggagttcattgcaccagttacactgaaggtctttgcaggctattatacgaagggatttgcactactgaattttgtagtaaaatactcccctgaacgacagcgttctcttcgtcctcatcatgatgcttctacatttaccataaacattgcacttaataacgtgggagaagactttcagggaggtggttgcaaatttctaaggtacaattgctctattgagtcaccacgaaaaggctggagcttcatgcatcctgggagactcacacatttgcatgaaggacttcctgttaaaaatggaacaagatacattgcagtgtcatttatagatccctaa

SEQ ID NO: 31 - DNA, 1482 bp, Homo sapiens
atggccctcttcggggccctcttcctagcgctgctggcaggcgcacatgcagagttcccaggctgcaagatccgcgtcacctccaaggcgctggagctggtgaagcaggaggggctgcgctttctggagcaagagctggagactatcaccattccggacctgcggggcaaagaaggccacttctactacaacatctctgaggtgaaggtcacagagctgcaactgacatcttccgagctcgatttccagccacagcaggagctgatgcttcaaatcaccaatgcctccttggggctgcgcttccggagacagctgctctactggttcttctatgatgggggctacatcaacgcctcagctgagggtgtgtccatccgcactggtctggagctctcccgggatcccgctggacggatgaaagtgtccaatgtctcctgccaggcctctgtctccagaatgcacgcggccttcgggggaaccttcaagaaggtgtatgattttctctccacgttcatcacctcagggatgcgcttcctcctcaaccagcagatctgccctgtcctctaccacgcagggacggtcctgctcaactccctcctggacaccgtgcctgtgcgcagttctgtggacgagcttgttggcattgactattccctcatgaaggatcctgtggcttccaccagcaacctggacatggacttccggggggccttcttccccctgactgagaggaactggagcctccccaaccgggcagtggagccccagctgcaggaggaagagcggatggtgtatgtggccttctctgagttcttcttcgactctgccatggagagctacttccgggcgggggccctgcagctgttgctggtgggggacaaggtgccccacgacctggacatgctgctgagggccacctactttgggagcattgtcctgctgagcccagcagtgattgactccccattgaagctggagctgcgggtcctggccccaccgcgctgcaccatcaagccctctggcaccaccatctctgtcactgctagcgtcaccattgccctggtcccaccagaccagcctgaggtccagctgtccagcatgactatggacgcccgtctcagcgccaagatggctctccgggggaaggccctgcgcacgcagctggacctgcgcaggttccgaatctattccaaccattctgcactggagtcgctggctctgatcccattacaggcccctctgaagaccatgctgcagattggggtgatgcccatgctcaatgagcggacctggcgtggggtgcagatcccactacctgagggcatcaactttgtgcatgaggtggtgacgaaccatgcgggattcctcaccatcggggctgatctccactttgccaaagggctgcgagaggtgattgagaagaaccggcctgctgatgtcagggcgtccactgcccccacaccgtccacagcagctgtctga

SEQ ID NO: 32 - DNA, 1674 bp, Homo sapiens
atggacggcattgtcccagatatagccgttggtacaaagcggggatctgacgagcttttctctacttgtgtcactaacggaccgtttatcatgagcagcaactcggcttctgcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggccttcatcgagatgaacacggaggaggctgccaacaccatggtgaactactacacctcggtgacccctgtgctgcgcggccagcccatctacatccagttctccaaccacaaggagctgaagaccgacagctctcccaaccaggcgcgggcccaggcggccctgcaggcggtgaactcggtccagtcggggaacctggccttggctgcctcggcggcggccgtggacgcagggatggcgatggccgggcagagccccgtgctcaggatcatcgtggagaacctcttctaccctgtgaccctggatgtgctgcaccagattttctccaagttcggcacagtgttgaagatcatcaccttcaccaagaacaaccagttccaggccctgctgcagtatgcggaccccgtgagcgcccagcacgccaagctgtcgctggacgggcagaacatctacaacgcctgctgcacgctgcgcatcgacttttccaagctcaccagcctcaacgtcaagtacaacaatgacaagagccgtgactacacacgcccagacctgccttccggggacagccagccctcgctggaccagaccatggccgcggccttcggtgcacctggtataatctcagcctctccgtatgcaggagctggtttccctcccacctttgccattcctcaagctgcaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagagagagtcacaccccaaagcctctttattcttttcggcgtctacggtgacgtgcagcgcgtgaagatcctgttcaataagaaggagaacgccctagtgcagatggcggacggcaaccaggcccagctggccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgccctcagtctccgaggaggatctcaaggtcctgttttccagcaatgggggcgtcgtcaaaggattcaagttcttccagaaggaccgcaagatggcactgatccagatgggctccgtggaggaggcggtccaggccctcattgacctgcacaaccacgacctcggggagaaccaccacctgcgggtctccttctccaagtccaccatctag

SEQ ID NO: 33 - DNA, 543 bp, Homo sapiens
atggcgcccggagtggcccgcgggccgacgccgtactggaggttgcgcctcggtggcgccgcgctgctcctgctgctcatcccggtggccgccgcgcaggagcctcccggagctgcttgttctcagaacacaaacaaaacctgtgaagagtgcctgaagaacgtctcctgtctttggtgcaacactaacaaggcttgtctggactacccagttacaagcgtcttgccaccggcttccctttgtaaattgagctctgcacgctggggagtttgttgggtgaactttgaggcgctgatcatcaccatgtcggtagtcgggggaaccctcctcctgggcattgccatctgctgctgctgctgctgcaggaggaagaggagccggaagccggacaggagtgaggagaaggccatgcgtgagcgggaggagaggcggatacggcaggaggaacggagagcagagatgaagacaagacatgatgaaatcagaaaaaaatatggcctgtttaaagaagaaaacccgtatgctagatttgaaaacaactaa

SEQ ID NO: 34 - DNA, 2484 bp, Homo sapiens
atggcctctgctgacaagaatggcgggagcgtgtcctctgtgtccagcagccgcctgcagagccggaagccacccaacctctccatcaccatcccgccacccgagaaagagacccaggcccctggcgagcaggacagcatgctgcctgagaggaagaacccagcctacttgaagagcgtcagcctccaggagccacgcagccgatggcaggagagttcagagaagcgccctggcttccgccgccaggcctcactgtcccagagcatccgcaagggcgcagcccagtggtttggagtcagcggcgactgggaggggcagcggcagcagtggcagcgccgcagcctgcaccactgcagcatgcgctacggccgcctgaaggcctcgtgccagcgtgacctggagctccccagccaggaggcaccgtccttccagggcactgagtccccaaagccctgcaagatgcccaagattgtggatccgctggcccggggccgggccttccgccacccggaggagatggacaggccccacgccccgcacccaccgctgacccccggagtcctgtccctcacctccttcaccagtgtccgttctggctactcccacctgccacgccgcaagagaatgtctgtggcccacatgagcttgcaagctgccgctgccctcctcaaggggcgctcggtgctggatgccaccggacagcggtgccgggtggtcaagcgcagctttgccttcccgagcttcctggaggaggatgtggtcgatggggcagacacgtttgactcctccttttttagtaaggaagaaatgagctccatgcctgatgatgtctttgagtcccccccactctctgccagctacttccgagggatcccacactcagcctcccctgtctcccccgatggggtgcaaatccctctgaaggagtatggccgagccccagtccccgggccccggcgcggcaagcgcatcgcctccaaggtgaagcactttgcctttgatcggaagaagcggcactacggcctcggcgtggtgggcaactggctgaaccgcagctaccgccgcagcatcagcagcactgtgcagcggcagctggagagcttcgacagccaccggccctacttcacctactggctgaccttcgtccatgtcatcatcacgctgctggtgatttgcacgtatggcatcgcacccgtgggctttgcccagcacgtcaccacccagctggtgctgcggaacaaaggtgtgtacgagagcgtgaagtacatccagcaggagaacttctgggttggccccagctcgattgacctgatccacctgggggccaagttctcaccctgcatccggaaggacgggcagatcgagcagctggtgctgcgcgagcgagacctggagcgggactcaggctgctgtgtccagaatgaccactccggatgcatccagacccagcggaaggactgctcggagactttggccacttttgtcaagtggcaggatgacactgggccccccatggacaagtctgatctgggccagaagcggacttcgggggctgtctgccaccaggaccccaggacctgcgaggagccagcctccagcggtgcccacatctggcccgatgacatcactaagtggccgatctgcacagagcaggccaggagcaaccacacaggcttcctgcacatggactgcgagatcaagggccgcccctgctgcatcggcaccaagggcagctgtgagatcaccacccgggaatactgtgagttcatgcacggctatttccatgaggaagcaacactctgctcccaggtgcactgcttggacaaggtgtgtgggctgctgcccttcctcaaccctgaggtcccagatcagttctacaggctctggctgtctctcttcctacatgctggcgtggtgcactgcctcgtgtctgtggtctttcaaatgaccatcctgagggacctggagaagctggccggctggcaccgtatcgccatcatcttcatcctcagtggcatcacaggcaacctcgccagtgccatctttctcccataccgggcagaggtgggcccggccggctcacagttcggcctcctcgcctgcctcttcgtggagctcttccagagctggccgctgctggagaggccctggaaggccttcctcaacctctcggccatcgtgctcttcctgttcatctgtggcctcctgccctggatcgacaacatcgcccacatcttcggcttcctcagtggcctgctgctggccttcgccttcctgccctacatcaccttcggcaccagcgacaagtaccgcaagcgggcactcatcctggtgtcactgctggcctttgccggcctcttcgccgccctcgtgctgtggctgtacatctaccccattaactggccctggatcgagcacctcacctgcttccccttcaccagccgcttctgcgagaagtatgagctggaccaggtgctgcactga

SEQ ID NO: 35 - DNA, 711 bp, Homo sapiens
atgctgagcaagggtctgaagcggaaacgggaggaggaggaggagaaggaacctctggcagtcgactcctggtggctagatcctggccacacagcggtggcacaggcacccccggccgtggcctctagctccctctttgacctctcagtgctcaagctccaccacagcctgcagcagagtgagccggacctgcggcacctggtgctggtcgtgaacactctgcggcgcatccaggcgtccatggcacccgcggctgccctgccacctgtgcctagcccacctgcagcccccagtgtggctgacaacttactggcaagctcggacgctgccctttcagcctccatggccagcctcctggaggacctcagccacattgagggcctgagtcaggctccccaacccttggcagacgaggggccaccaggccgtagcatcgggggagcagcgcccagcctgggtgccttggacctgctgggcccagccactggctgtctactggacgatgggcttgagggcctgtttgaggatattgacacctctatgtatgacaatgaactttgggcaccagcctctgagggcctcaaaccaggccctgaggatgggccgggcaaggaggaagctccggagctggacgaggccgaattggactacctcatggatgtgctggtgggcacacaggcactggagcgaccgccggggccagggcgctga

SEQ ID NO: 36 - DNA, 735 bp, Homo sapiens
atggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggtgggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggtgagttcaggacatctcgggagcacccccaccccttcaccactacccttggccccctccttgtgttccaaagccaccaccacaggacaggactttctgcagactggtctcttctaacaggctggatgtccttggggggcccatcctgtcccgagccaacatag

SEQ ID NO: 37 - DNA, 1311 bp, Homo sapiens
atggatcagccttttactgtgaattctctgaaaaagttagctgctatgcctgaccatacagatgtttccctaagcccagaagagcgagtccgtgccctaagcaagcttggttgtaatatcaccatcagtgaagacatcactccacgacgttactttaggtctggagtagagatggagaggatggcgtctgtgtatttggaagaaggaaatttggaaaatgcctttgttctttataataaatttataaccttatttgtagaaaagcttcctaaccatcgagattaccagcaatgtgcagtacctgaaaagcaggatattatgaagaaactgaaggagattgcattcccaaggacagatgaattgaaaaacgaccttttaaagaaatataacgtagaataccaagaatatttgcaaagcaaaaacaaatataaagctgaaattctcaaaaaattggagcatcagagattgatagaggcagaaaggaagcggattgctcagatgcgccagcagcagctagaatcggagcagtttctgtttttcgaagatcaactcaagaagcaagagttagcccgaggtcaaatgcgaagtcagcaaacctcagggctgtcagagcagattgatgggagcgctttgtcctgcttttccacacaccagaacaattccttgctgaatgtatttgcagatcaacctaataaaagtgatgcaaccaattatgctagccactctcctcctgtaaacagggccttaacaccagctgctactctaagtgctgttcagaatttagtggttgaaggactgcgatgtgtagttttgccagaagatctttgccacaaatttctgcaactggcagaatctaatacagtgagaggaatagaaacctgtggaatactctgtggaaaactgacacataatgaatttactattacccatgtaattgtgccaaagcagtctgcgggaccagactattgtgacatggagaatgtagaggaattattcaatgttcaggatcaacatgatctcctcactctaggatggatccatacacatcccactcaaactgcatttttatccagcgttgatcttcacactcactgttcctatcaactcatgttgccagaggccattgccattgtttgctcaccaaagcataaagacactggcatcttcaggctcaccaatgctggcatgcttgaggtttctgcttgtaaaaaaaagggctttcatccacacaccaaggagcccaggctgttcagtatatgcaaacatgtgttggtaaaagacataaaaataattgtgttggatctgaggtga

SEQ ID NO: 38 - DNA, 564 bp, Homo sapiens
atgggcgctgcccactccgcgtctgaggaggtgcgggagctcgagggcaagaccggcttctcatcggatcagatcgagcagctccatcggagatttaagcagctgagtggagatcagcctaccattcggaacctgcgcaagggacccagtggcctggctgatgagatcaatttcgaggacttcctgaccatcatgtcctacttccggcccatcgacaccaccatggacgaggaacaggtggagctgtcccggaaggagaagctgagatttctgttccacatgtacgactcggacagcgacggccgcatcactctggaagaatatcgaaatgtggtcgaggagctgctgtcgggaaaccctcacatcgagaaggagtccgctcgctccatcgccgacggggccatgatggaggcggccagcgtgtgcatggggcagatggagcctgatcaggtgtacgaggggatcaccttcgaggacttcctgaagatctggcaggggatcgacattgagaccaagatgcacgtccgcttccttaacatggaaaccatggccctctgccactga

SEQ ID NO: 39 - DNA, 624 bp, Homo sapiens
atggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctga

SEQ ID NO: 40 - DNA, 2697 bp, Homo sapiens
atggtgtgggaccggcaaaccaagatggagtatgagtggaaacctgacgagcaagggcttcagcaaatcctgcagctgttgaaggagtcccagtccccagacaccaccatccagagaaccgtgcaacaaaaactggaacaacttaatcagtatccagactttaacaactacttgatttttgttcttacaaaattaaaatctgaagatgaacccacaagatcattgagtggtcttatcttgaagaataatgtgaaagcacactttcagaacttcccaaatggtgtaacagactttattaaaagtgaatgtttaaataatattggtgactcctctcctctgattagagccactgttggtattttgatcacaactatagcctccaagggagaattgcagaattggcctgacctcttaccaaaactctgtagcctgttggattctgaagattataatacctgtgagggagcatttggtgcccttcagaagatttgtgaagattctgctgagattttagacagtgatgttttagatcgtcctctcaacatcatgattcccaaatttttacagttcttcaagcatagtagtccaaaaataaggtctcacgctgttgcatgtgtcaatcagtttatcatcagtaggactcaagctctaatgttgcacattgattcttttattgagaatctctttgcattagctggtgatgaagaaccagaggtacggaaaaatgtgtgccgagcacttgtgatgttgctcgaagttcgaatggatcgcctgcttcctcacatgcataatatagttgagtacatgctacagaggactcaagatcaagatgaaaatgtggctttagaagcctgtgaattttggctaactttagctgaacagccaatatgcaaagatgtactcgtaaggcatcttcctaagttgattcctgtgttagtgaatggcatgaagtactcagacatagatattatcctacttaagggtgatgttgaagaagacgaaacgattcctgatagtgaacaggatatacggccacgttttcaccgatcgaggacggtggctcagcagcatgatgaagatggaattgaagaggaagatgatgatgatgatgaaattgatgatgatgatacaatttctgactggaatctaagaaaatgttctgctgctgccctggatgttcttgcaaatgtgtatcgtgatgaactgctgccacatattttgccccttttgaaagaattactttttcatcatgaatgggttgttaaagaatcaggcattttggttttaggagcaattgctgaaggttgcatgcagggcatgattccatacttgcctgagcttattcctcaccttattcagtgcctctctgataaaaaggctcttgtgcgttccataacatgctggactcttagccgctatgcacactgggtggtcagccagccgccagacacgtacctgaagccattaatgacagaattgctaaagcgcatcctggacagcaacaagagagtacaagaagctgcctgcagtgcctttgctaccctagaagaggaggcttgtacagaacttgttccttaccttgcttatatacttgataccctggtctttgcatttagtaaataccagcataagaacctgctcattctttacgatgccataggaacattagcagattcagtaggacatcatttaaacaaaccagaatatattcagatgctaatgcctccactgatccagaaatggaacatgttaaaggatgaagataaagatctcttccctttacttgagtgcctatcttcagttgccacagcactgcagtctggattccttccgtactgtgaacctgtgtatcagcgttgtgtaaacctagtacagaagactcttgcacaagccatgctaaacaatgctcaaccagatcaatatgaagctccagataaagattttatgatagtggctcttgatttactgagtggcctggctgaaggacttggaggcaacattgaacagctggtagcccgaagtaacatcctgacactaatgtatcagtgcatgcaggataaaatgccagaagttcgacagagttcttttgccctgttaggtgacctcacaaaagcttgctttcagcatgttaagccttgtatagctgatttcatgccaatattgggaaccaacctaaatccagaattcatttcagtctgcaacaatgccacatgggcaattggagaaatctccattcaaatgggtatagagatgcagccttatattcctatggtgttgcaccagcttgtagaaatcattaacagacccaacacaccaaagacgttgttagagaatacagcaataacaattggtcgtcttggttacgtttgtcctcaagaggtggcccccatgctacagcagtttataagaccctggtgcacctctctgagaaacataagagacaatgaggaaaaggattcagcattccgtggaatttgtaccatgatcagtgtgaatcccagtggcgtaatccaagattttatatttttttgtgatgccgttgcatcatggattaacccaaaagatgatctcagagacatgttctgtaagatccttcatggatttaaaaatcaagttggcgatgaaaattggaggcgtttctctgaccagtttcctcttcccttaaaagagcgtcttgcagctttttatggtgtttaa

SEQ ID NO: 41 - DNA, 1176 bp, Homo sapiens
atggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtga

SEQ ID NO: 42 - DNA, 357 bp, Homo sapiens
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaatctacttttttacttga

SEQ ID NO: 43 - DNA, 642 bp, Homo sapiens
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattga

SEQ ID NO: 44 - DNA, 1017 bp, Homo sapiens
atgaccaccaccctcgtgtctgccaccatcttcgacttgagcgaagttttatgcaagggtaacaagatgctcaactatagtgctcccagtgcagggggttgcctgctggacagaaaggcagtgggcacccctgctggtgggggcttccctcggaggcactcagtcaccctgcccagctccaagttccaccagaaccagctcctcagcagcctcaagggtgagccagcccccgctctgagctcgcgagacagccgcttccgagaccgctccttctcggaagggggcgagcggctgctgcccacccagaagcagcccgggggcggccaggtcaactccagccgctacaagacggagctgtgccgcccctttgaggaaaacggtgcctgtaagtacggggacaagtgccagttcgcacacggcatccacgagctccgcagcctgacccgccaccccaagtacaagacggagctgtgccgcaccttccacaccatcggcttttgcccctacgggccccgctgccacttcatccacaacgctgaagagcgccgtgccctggccggggcccgggacctctccgctgaccgtccccgcctccagcatagctttagctttgctgggtttcccagtgccgctgccaccgccgctgccaccgggctgctggacagccccacgtccatcaccccaccccctattctgagcgccgatgacctcctgggctcacctaccctgcccgatggcaccaataacccttttgccttctccagccaggagctggcaagcctctttgcccctagcatggggctgcccgggggtggctccccgaccaccttcctcttccggcccatgtccgagtcccctcacatgtttgactctccccccagccctcaggattctctctcggaccaggagggctacctgagcagctccagcagcagccacagtggctcagactccccgaccttggacaactcaagacgcctgcccatcttcagcagactttccatctcagatgactaa

SEQ ID NO: 45 - DNA, 18 bp, Artificial Sequence
  Description of Artificial Sequence Synthetic primer
taccccttcaacgccaag

SEQ ID NO: 46 - DNA, 20 bp, Artificial Sequence
  Description of Artificial Sequence Synthetic primer
cctggaaccgttctttcagt

SEQ ID NO: 47 - DNA, 20 bp, Artificial Sequence
  Description of Artificial Sequence Synthetic primer
ctgtaggagctggagtggga

SEQ ID NO: 48 - DNA, 20 bp, Artificial Sequence
  Description of Artificial Sequence Synthetic primer
ttcttcctttcgacccattg

SEQ ID NO: 49 - DNA, 19 bp, Artificial Sequence
  Description of Artificial Sequence Synthetic primer
gacaagttttggtggcacg

SEQ ID NO: 50 - DNA, 20 bp, Artificial Sequence
  Description of Artificial Sequence Synthetic primer
cacgtggaatacacctgcaa

SEQ ID NO: 51 - DNA, 19 bp, Artificial Sequence
  Description of Artificial Sequence Synthetic primer
accgaggcactcagaggag

SEQ ID NO: 52 - DNA, 19 bp, Artificial Sequence
  Description of Artificial Sequence Synthetic primer
gccattagcgcatcacagt

SEQ ID NO: 53 - DNA, 20 bp, Artificial Sequence
  Description of Artificial Sequence Synthetic primer
acagtgggggtgtacgagtc

SEQ ID NO: 54 - DNA, 21 bp, Artificial Sequence
  Description of Artificial Sequence Synthetic primer
gatgtcatcctcctcctcctc

SEQ ID NO: 55 - DNA, 20 bp, Artificial Sequence
  Description of Artificial Sequence Synthetic primer
aatgagctggaagatgacgc

SEQ ID NO: 56 - DNA, 20 bp, Artificial Sequence
  Description of Artificial Sequence Synthetic primer
gaaggtcagcttgtatcggc

SEQ ID NO: 57 - DNA, 20 bp, Artificial Sequence
  Description of Artificial Sequence Synthetic primer
atgtcgtctggtccctgttc

SEQ ID NO: 58 - DNA, 19 bp, Artificial Sequence
  Description of Artificial Sequence Synthetic primer
ccaaccatcttcctgtccc

SEQ ID NO: 59 - DNA, 20 bp, Artificial Sequence
  Description of Artificial Sequence Synthetic primer
caggacctgctcaatgtcaa

SEQ ID NO: 60 - DNA, 20 bp, Artificial Sequence
  Description of Artificial Sequence Synthetic primer
caaagccaatccgacactct

SEQ ID NO: 61 - DNA, 21 bp, Artificial Sequence
  Description of Artificial Sequence Synthetic primer
ggaactagggggaaaatcaca

SEQ ID NO: 62 - DNA, 19 bp, Artificial Sequence
  Description of Artificial Sequence Synthetic primer
gtcaaagggcatctcccat

SEQ ID NO: 63 - DNA, 21 bp, Artificial Sequence
  Description of Artificial Sequence Synthetic primer
ggtcgtgtttccaaagagaaa

SEQ ID NO: 64 - DNA, 21 bp, Artificial Sequence
  Description of Artificial Sequence Synthetic primer
tgcagccaatattctcctctc

SEQ ID NO: 65 - DNA, 17 bp, Artificial Sequence
  Description of Artificial Sequence Synthetic primer
cagcctccggagaggaa

SEQ ID NO: 66 - DNA, 20 bp, Artificial Sequence
  Description of Artificial Sequence Synthetic primer
cagtcccttctgtcccttca

SEQ ID NO: 67 - DNA, 21 bp, Artificial Sequence
  Description of Artificial Sequence Synthetic primer
tcagaccaatgccagaggagc

SEQ ID NO: 68 - DNA, 22 bp, Artificial Sequence
  Description of Artificial Sequence Synthetic primer
gctgtctgttgagggaaagctg

SEQ ID NO: 69 - DNA, 23 bp, Artificial Sequence
  Description of Artificial Sequence Synthetic primer
gtcctggagatggaatacagatc

SEQ ID NO: 70 - DNA, 21 bp, Artificial Sequence
  Description of Artificial Sequence Synthetic primer
ctgacaagccactgctagcac

SEQ ID NO: 71 - DNA, 22 bp, Artificial Sequence
  Description of Artificial Sequence Synthetic primer
cagcaagatgaagagcgacgtc

SEQ ID NO: 72 - DNA, 21 bp, Artificial Sequence
  Description of Artificial Sequence Synthetic primer
catgtcctgctctgagctctc

SEQ ID NO: 73 - DNA, 21 bp, Artificial Sequence
  Description of Artificial Sequence Synthetic primer
agcatccacctgagcttcctg

SEQ ID NO: 74 - DNA, 22 bp, Artificial Sequence
  Description of Artificial Sequence Synthetic primer
ctggcagaaaggatgatgaccc

SEQ ID NO: 75 - DNA, 22 bp, Artificial Sequence
  Description of Artificial Sequence Synthetic primer
ttcctgccaacaagacacggtg

SEQ ID NO: 76 - DNA, 22 bp, Artificial Sequence
  Description of Artificial Sequence Synthetic primer
agaccaatgaaccagccctgag

SEQ ID NO: 77 - DNA, 24 bp, Artificial Sequence
  Description of Artificial Sequence Synthetic primer
atgctgggactcatgtatgtctac

SEQ ID NO: 78 - DNA, 27 bp, Artificial Sequence
  Description of Artificial Sequence Synthetic primer
cttctcatactgtgaattgttcttgac

SEQ ID NO: 79 - DNA, 23 bp, Artificial Sequence
  Description of Artificial Sequence Synthetic primer
gtcgggatataagcccagaagag

SEQ ID NO: 80 - DNA, 24 bp, Artificial Sequence
  Description of Artificial Sequence Synthetic primer
caggagtctctgaagaaatttccg

(19) United States
(12) Patent Application Publication
(10) Pub. No.: US 2014/0194310 A1
(43) Pub. Date: Jul. 10, 2014

(54) GENES DYSREGULATED IN AUTISM AS BIOMARKERS AND TARGETS FOR THERAPEUTIC PATHWAYS

(71) Applicants: Daniel H. Geschwind, Santa Monica, CA (US); Irina Voineagu, Los Angeles, CA (US)

(72) Inventors: Daniel H. Geschwind, Santa Monica, CA (US); Irina Voineagu, Los Angeles, CA (US)

(21) Appl. No.: 14/119,755

(22) PCT Filed: May 24, 2012
(86) PCT No.: PCT/US12/39269

§ 371 Date: Jan. 27, 2014

Related U.S. Application Data
(60) Provisional application No. 61/489,471, filed on May 24, 2011.

(51) Int. Cl. C12Q 1/68

(52) U.S. Cl. CPC C12Q 1/6883
USPC 506/9; 435/6.12

(57) ABSTRACT

The disclosed invention comprises methods and materials for screening cells for genetic profiles associated with autism spectrum disorders. The methods typically involve isolating a cell from an individual and then observing the expression profile of one or more genes in the cell, wherein certain expression patterns of the genes observed are associated with autism spectrum disorders.

### REFERENCE TO RELATED APPLICATIONS

This application claims priority under Section 119(e) from U.S. Provisional Application Ser. No. 61/489,471, filed May 24, 2011, the contents of which are incorporated herein by reference.

### STATEMENT OF GOVERNMENT SUPPORT

This invention was made with Government support of Grant No. MH081754, awarded by the National Institutes of Health. The Government has certain rights in this invention.

### FIELD OF THE INVENTION

The invention relates to methods and materials for observing gene expression profiles that are associated with conditions such as autism.

### BACKGROUND OF THE INVENTION

Autism comprises a behaviorally defined spectrum of disorders characterized by impairment of social interaction, deficiency or abnormality of speech development, and limited activities and interest. To standardize the diagnosis of autism spectrum disorders (ASD), diagnostic criteria have been defined by the World Health Organization (International Classification of Diseases, 10th Revision (ICD-10), 1992) and the American Psychiatric Association (Diagnostic and Statistical Manual of Mental Disorders, 4th edition, Text Revision. Washington D.C., American Psychiatric Association, 2000 (DSM-IV)).

Genetic factors are significant determinants of autism spectrum disorders (see, e.g. Geschwind et al., (2007), Curr Opin Neurobiol, 17, 103-11). It has been shown that individuals with ASD carry chromosomal abnormality at a greater frequency than the general population (see, e.g. Veenstra-Vanderweele et al., (2004), Annu Rev Genomics Hum Genet, 5, 379-405; Vorstman et al., (2006), Mol Psychiatry, 11, 1, 18-28; Jacquemont et al. (2006), J Med Genet, 43, 843-9; Szatmari et al. (2007), Nat Genet, 39, 319-28; Sebat et al. (2007), Science, 316, 445-9). Maternally inherited duplication of 15q11-13 (dup15q) is the most common chromosomal abnormality in ASD. Over-expression of genes located in the duplicated region, including cytoplasmic FMR1 interacting protein 1 (CYFIPI), was shown in lymphoblastoid cell lines from ASD with dup15q (see, e.g. Nishimura et al. (2007), Hum Mol Genet, 16, 1682-98). A cryptic deletion located at the boundary of the first exon and first intron of ataxin-2 binding protein-1 (A2BP1) was identified in a female with ASD, resulting reduced mRNA expression in the individual's lymphocytes (see, e.g. Martin et al. (2007), Am J Med Genet B Neuropsychiatr Genet). Loss of copy number of neurexin 1 (NRXN1) was identified in two females sibs with ASD but not in either parent (see, e.g. Szatmari et al. (2007), Nat Genet, 39, 319-28). Loss of copy number and decreased expression of SH3 and multiple ankyrin repeat domains 3 (SHANK3) were identified in four individuals with ASD (see, e.g. Jeffries et al., (2005), Am J Med Genet A, 137, 139-47; and Durand et al. (2007), Nat Genet, 39, 25-7). A common ‘C’ allele in the promoter region of met proto-oncogene (MET) has also been shown to have a strong association with ASD (see, e.g. Campbell et al., (2006), Proc Natl Acad Sci USA, 103, 16834-9). The ‘C’ variant causes a twofold decrease in MET promoter activity. Such findings provide evidence that dysregulation of gene expression may affect susceptibility to and/or cause ASD.

Transcriptome profiling using DNA microarray represents an efficient manner in which to uncover an unanticipated relationship between gene expression alterations and neuropsychiatric diseases (see, e.g. Geschwind, D. H. (2003), Lancet Neurol, 2, 275-82; and Mimics et al., (2006), Biol Psychiatry, 60, 163-76). Several studies have suggested that blood-derived cells can be used to identify candidate genes in neuropsychiatric diseases, including ASD. Hu et al. analyzed gene expression profiling of lymphoblastoid cells from monozygotic (MZ) twins discordant in severity of ASD (see, e.g. Hu et al., (2006), BMC Genomics, 7, 118). Several genes were differentially expressed between MZ twins, suggesting candidate genes for ASD may be differentially expressed in lymphoblastoid cells from individuals with ASD. Previously analyses include genome-wide expression profiles of lymphoblastoid cells from ASD with full mutation of FMR1 (FMR1-FM) or dup15q, each of which account for 1-2% of ASD cases in large series, and non-autistic controls (see, e.g. Nishimura et al. (2007), Hum Mol Genet, 16, 1682-98). The gene expression profiles clearly distinguished ASD from controls and separated individuals with ASD based on their genetic etiology. The expression profiles also revealed shared pathways between ASD with FMR1-FM and ASD with dup15q.

While progress in understanding genetic factors associated with autism spectrum disorders has been made, specific assays for constellations of genetic factors associated with autism spectrum disorders would be a significant benefit to medical personnel. Tests for genetic factors associated with autism spectrum disorders are valuable for the diagnosis of this syndrome, as well as useful for research on the genetic mechanisms involved in autism spectrum disorders. Moreover, while there is no known medical treatment for autism, success has been reported for early intervention with behavioral therapies. In this context, such assays would facilitate the early identification of the disease, one now typically diagnosed between ages three and five. The increasing prevalence of autism spectrum disorders over recent years as well as the lack of effective means of genetic diagnosis, prevention or treatment make identifying ASD biomarkers and defining molecular pathways implicated in ASD a compelling goal.

### SUMMARY OF THE INVENTION

Autism spectrum disorder is a heterogeneous condition and is likely to result from the combined effects of multiple, subtle genetic changes interacting with environmental factors. The disclosure provided herein characterizes genome-wide expression profiles of postmortem brain tissue from several brain regions in ASD patients and controls in order to identify genes showing consistent changes in mRNA levels in ASD brain. The ASD brain transcriptome is further analyzed using a network-based approach (co-expression network analysis), to identify groups of functionally related genes that are dysregulated at a transcriptional level in ASD brain. The experimental data presented herein highlights genes that are dysregulated at a transcriptional level in ASD in disease-relevant tissue and further defines sets of co-expressed genes that are useful as biomarkers for ASD, as well as being targets for therapeutic interventions.

The invention disclosed herein has a number of embodiments. Illustrative embodiments of the invention include methods of identifying a human cell having a gene expression profile associated with autism spectrum disorders by observing the expression of at least one gene in a test human cell, where the expression of that gene is observed to be dysregulated in individuals diagnosed with autism spectrum disorders (e.g. one or more of the genes disclosed in Tables A and B below). An illustrative embodiment of the invention is a method of identifying a test mammalian cell as having a gene expression profile observed in individuals diagnosed with autism by observing the expression of at least one gene comprising a sequence selected from the group consisting of SEQ ID NOs: 1-44 in the test mammalian cell in order to see if the test cell has a gene expression profile that is observed in individuals diagnosed with autism. In such methods, one can, for example, determine if levels of mRNA expression are at least two standard deviations away from levels of mRNA expression that are commonly observed in individuals not affected with autism. Alternatively in such methods, one can, for example, determine if the levels of mRNA expression are at least 20, 30, 40, 50, 60 or 70% above or below the levels of mRNA expression that are commonly observed in individuals not affected with autism.

In certain embodiments, methods of the invention are used to facilitate the diagnosis of an autism spectrum disorder. For example, in some embodiments of the invention, the cellular gene expression examined by such methods is that found in a test cell obtained from an individual identified as being predisposed to and/or exhibiting a behavior associated with autism spectrum disorders. Typically this cellular gene expression is compared to cellular gene expression in a control cell, for example, one obtained from an individual previously identified as not being predisposed to and/or exhibiting a behavior associated with autism spectrum disorders. In certain embodiments, the test cell examined by this method and the control cell are obtained from individuals who are related as siblings or as a parent and a child. Typically one or more cells used in these methods are leukocytes obtained from the peripheral blood.

In illustrative methods for observing an expression profile of one or more genes, mRNA expression is observed, for example by using a using quantitative PCR (qPCR) technique. In certain embodiments of the invention, the expression profile of the genes in is observed using a microarray of polynucleotides. Alternatively, polypeptide expression is observed and quantified, for example by using an antibody specific for a polypeptide encoded by a gene whose expression is shown to be dysregulated in autism spectrum disorders (e.g. using an ELISA technique or the like). Alternatively, the expression profile of a gene is observed using a single nucleotide polymorphism (SNP) detection or Southern blotting technique (e.g. to identify polymorphisms, deletions and/or duplications in genomic sequences).

Embodiments of the invention include kits comprising, for example, a first container, a label on said container, and a composition contained within said container; wherein the composition includes polymerase chain reaction (PCR) primer effective in the quantitative real time analysis of the mRNA expression levels of one or more genes disclosed herein whose expression is shown to be dysregulated in autism spectrum disorders (e.g. one or more of the 444 genes identified herein such as those disclosed in Table A or B); the label on said container, or a package insert included in said container indicates that the composition can be used to observe expression levels of these genes in at least one type of human leukocyte; a second container comprising a pharmaceutically-acceptable buffer; and instructions for using the PCR primer to obtain an expression profile of the one or more genes. Optionally the kit comprises 2, 3, 4, 5, 6, 7, 8, 9 or 10 polymerase chain reaction (PCR) primers effective in the quantitative real time analysis of the mRNA expression levels of different genes disclosed in the Tables below.

In some embodiments of the invention, one can observe an expression profile of at least, 2, 3, 4, 5, 6, 7, 8, 9 or 10 or more genes whose expression is shown to be dysregulated in autism spectrum disorders (e.g. using microarray technologies). In certain embodiments of the invention, the method is performed on a plurality of individuals and the results are then categorized based upon similarities or differences in their gene expression profiles. Optionally, the expression profile(s) is observed and/or collected and/or stored using a computer system comprising a processor element and a memory storage element adapted to process and store data from one or more expression profiles (e.g. in a library of such profiles). In this context, certain embodiments of the invention comprise an electronically searchable library of profiles, wherein the profiles include an individual's gene expression data in combination with other diagnostic data, for example assessments of behavior associated with autism spectrum disorders.

Other embodiments of this invention comprise methods of screening compounds that can modulate the mRNA and/or protein expression of a gene disclosed herein (e.g. those disclosed in Table A or B). Illustrative methods can include the steps of contacting a cell that expresses an endogenous or exogenous mRNA and/or protein with one or more test compounds and then determining if the one or more compounds modulates mRNA and/or protein expression in the cell (e.g. by qPCR techniques practiced on the cell in the presence and absence of the one or more compounds). A related embodiment of this invention comprises a method of screening compounds that interact with an mRNA or protein of a gene disclosure herein. Illustrative methods can include the steps of contacting one or more compounds with the mRNA or protein, and then determining if a compound interacts with the mRNA or protein (e.g. by binding techniques that separating compounds that interact with the mRNA or protein from compounds that do not).

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIG. 1 shows a diagram depicting a number of genes showing significant expression differences between frontal and temporal cortex in control samples (top) and autism samples (bottom) at FDR <0.05 (left). Top 20 genes differentially expressed between frontal and temporal cortex in control samples (right). All of the genes shown are also differentially expressed between frontal and temporal cortex in fetal midgestation brain (see, e.g. Johnson, M. B. et al. Neuron 62, 494-509 (2009), but show no significant expression differences between frontal and temporal cortex in autism. The horizontal bars depict P values for differential expression between frontal and temporal cortex in the autism and control groups.

FIG. 2 shows A2BP1-dependent differential splicing events. (A) Top A2BP1-specific differential splicing events. Differential splicing events showing the most significant differences in alternative splicing between low-A2BP1 autism cases and controls as well as differential splicing differences consistent with the A2BP1 binding site position. The horizontal axis depicts the percentage of transcripts including the alternative exon. Lower Bar, autism samples; Top Bar, control samples. (B) Relevant gene ontology categories enriched in the set of genes containing exons differentially spliced between low-A2BP1 autism cases and controls.

FIG. 3 shows data illustrating the quantitative RT-PCR validation of DE genes. Average fold changes in the autism group (n>=5) relative to matched controls (n>=5) are shown (y-axis) for genes whose expression changes detected by arrays were validated by qRT-PCR. Top-upregulated genes, Bottom-downregulated genes. Error bars represent standard deviation of the mean.

FIG. 4 provides normalized expression values and ratios of temporal to frontal expression levels for selected genes showing attenuation of regional gene expression in ASD. FC•fold change, AF-ASD frontal cortex, AT-ASD temporal cortex, CF control frontal cortex, CT•control temporal cortex. Frontal and temporal cortex from the same brain are connected by a line.

FIG. 5 provides data showing A2BP1 expression values and A2BP1-dependent differential splicing events. (A) A2BP1 expression values as measured by microarrays. Expression values averages for two probes with highest expression level are plotted for both ASD and control groups. Black lines mark the mean and standard deviations from the mean. Smaller grey lines-Control samples used for RNA seq, “*”-ASD samples used for RNA seq. “**”-independent ASD samples used for RT PCR validation of DS events. (B) Semiquantatitive RT-PCR validation of DS events. A-pooled ASD samples (n=2-3), C-pooled control samples (n=2-3). Top panel: validation of DS events using the samples analyzed by RNA-seq. panel validation of DS events using additional independent ASD cases with low A2BP1 levels (shown as “*” in (A)).

FIGS. 6A-6I provide a Table showing GWAS p-values for genes in the M12 module, for which a SNP was mapped (see methods in the Example below) and the associated P-value was available (see, also Wang et al., Am. J. Hum. Genet. 81, 1278-1283 (2007) and/or Wang, K. et al. Nature 459, 528-533 (2009)).

FIG. 7 shows an embodiment of an illustrative computer system that can be used with embodiments of the invention.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. It must also be noted that as used herein and in the appended claims, the singular forms “a”, “and”, and “the” include plural referents unless the context clearly dictates otherwise. All numbers recited in the specification and associated claims that refer to values that can be numerically characterized with a value other than a whole number (e.g. a number of standard deviations from a mean) are understood to be modified by the term “about”.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

### ILLUSTRATIVE EMBODIMENTS OF THE INVENTION

Autism is part of a spectrum of disorders including Asperger syndrome (AS) and other pervasive developmental disorders (PPD). The term “autism” is used herein according to its art accepted meaning and encompasses conditions of impaired social interaction and communication with restricted repetitive and stereotyped patterns of behavior, interests and activities present before the age of 3, to the extent that health may be impaired. AS is typically distinguished from other autistic disorders by a lack of a clinically significant delay in language development in the presence of the impaired social interaction and restricted repetitive behaviors, interests, and activities that characterize the autism-spectrum disorder (ASD). PPD-NOS (PPD, not otherwise specified) is typically used to categorize children who do not meet the strict criteria for autism but who come close, either by manifesting atypical autism or by nearly meeting the diagnostic criteria in two or three of the key areas.

The disclosure provided herein identifies genes that are observed to be Dysregulated in Autism Spectrum Disorders (e.g. autism as diagnosed by an Autism Diagnostic Interview (ADI-R), an Autism Diagnostic Observation Schedule (ADOS), an IQ surrogate test based on Raven's Progressive Matrices, observations of restricted repetitive behaviors or speech delay or the like). In the instant disclosure, these genes are collectively referred to as “DASD genes” for purposes of convenience. Human DASD genes useful in embodiments of the invention are shown for example in the Tables and Figures provided herein. Voineagu et al., Nature 474, 380-384 (2011) (the contents of which are incorporated by reference) includes illustrative information relating to these genes. Because the genes disclosed herein are known in the art and further because of the high level of skill possessed by artisans in this technical field, the information as disclosed herein places artisans in possession of the polynucleotide and polypeptide sequences of these genes by providing them with the specific disclosure which allows them to retrieve this sequence information from library sources such as GenBank and/or UniProtKB/Swiss-Prot with only minimal effort. As is know in the art, GenBank® is the NIH genetic sequence database, an annotated collection of all publicly available DNA sequences; and UniProtKB/Swiss-Prot is a curated protein sequence database which provides a high level of annotation (e.g. technical references describing the features of these genes), a minimal level of redundancy and high level of integration with other databases. The DASD gene polynucleotide and polypeptide sequence information can be retrieved from GenBank and/or UniProtKB/Swiss-Prot library databases by, for example, querying these databases using the DASD disclosure information as provided herein and/or incorporated by reference into the instant specification (e.g. the gene name, gene symbol, gene RefSeq number, gene locus etc.).

As disclosed in detail below, this disclosure provides methods and materials that can be used in the diagnosis and treatment of autism spectrum disorders, and autism-associated disorders. In typical embodiments of the invention one observes an expression profile of at least one gene disclosed herein, wherein a dysregulated expression profile provides evidence of an autism spectrum disorder. Embodiments of invention can be used for example in the diagnosis of (including a predisposition to), and/or treatment of autism spectrum disorders such as Asperger syndrome, pervasive developmental disorder, mental retardation, speech delay, and other associated psychiatric and neurological phenomena.

The invention disclosed herein has a number of embodiments. One embodiment is a method of identifying an individual having a gene expression profile associated with autism spectrum disorders comprising: observing an expression profile of one or more DASD genes in a test cell obtained from the individual (e.g. mRNA expression in a peripheral blood leukocyte obtained from an individual suspected of having a form of autism); wherein a determination that the expression of one (or more) DASD genes in the test cell exhibits a statistically significant difference from the expression of these DASD gene(s) as observed in control cell(s) (e.g. mRNA expression in a peripheral blood leukocyte obtained from a non-effected sibling) identifies the test cell as having a gene expression profile associated with autism spectrum disorders. In certain embodiments of the invention, the dysregulation of a group of genes and/or a specific pattern of changes in their expression (e.g. certain genes being overexpressed and other genes being under expressed) in such test cells as compared to control cells is used to characterize autism and/or identify individuals who have a high probability of having an ASD.

In typical embodiments of the invention, the gene expression profile comprises data relating to the levels of mRNA expressed by one or more DASD genes in the cell. In embodiments of the invention, gene expression can be qualified or quantified using a comparison of expression in a test cell relative to a mean expression observed in a control cell. For example, in some embodiments of the invention, mRNA expression levels of one or more DASD gene(s) in a test cell that is/are at least one, two, three, four or five standard deviations from the mean mRNA expression level(s) of these gene(s) as observed in control cell(s) identifies the test cell as having an expression profile associated with autism spectrum disorders. In other embodiments of the invention, mRNA expression levels of one or more DASD gene(s) in a test cell that is/are at least at least 20, 30, 40, 50, 60 or 70% above or below the mean mRNA expression level(s) of these gene(s) as observed in control cell(s) identifies the test cell as having an expression profile associated with autism spectrum disorders.

Typically in such methods of observing an expression profile of a DASD gene, mRNA expression is observed, for example by using a using quantitative PCR (qPCR) technique. In certain embodiments of the invention, the expression profile of the DASD gene in the test cell is observed using a microarray of polynucleotides. Alternatively, DASD polypeptide expression is observed, for example by using an antibody specific for a polypeptide encoded by a DASD gene (e.g. using an ELISA technique or the like). Alternatively, the expression profile is observed using Southern blotting (e.g. to identify deletions in or duplications of DASD genomic sequences).

Autism spectrum disorder is a heterogeneous condition that appears to result from the combined effects of multiple, subtle genetic changes interacting with environmental factors. Consequently, in some embodiments of the invention, an expression profile of at least, 2, 3, 4, 5, 6, 7, 8, 9 or 10, 15, 20, 25, 30, 35, 40 or more DASD genes are observed in order to obtain a detailed profile of these multiple genetic changes and/or to stratify individuals into subsets of autism spectrum disorders (e.g. using microarray technologies). For example, in certain embodiments of the invention, the method is performed on a plurality of individuals and then segregated based upon similarities or differences in their gene expression profiles. Optionally, the expression profile(s) of the test mammalian cell is observed using a computer system comprising a processor element and a memory storage element adapted to process and store data from one or more expression profiles (e.g. in a library of such profiles). In this context, one embodiment of the invention comprises an electronically searchable library of profiles, wherein the profiles include individual's gene expression data in combination with other diagnostic data, for example assessments of whether the individual exhibits behavior associated with an autism spectrum disorder (e.g. behavioral test data such as that obtained in an Autism Diagnostic Interview (ADI-R)).

In typical embodiments of the invention, these methods are used to facilitate diagnosis of an autism spectrum disorder in an individual. In this context, a cell examined in the methods of the invention can be a leukocyte obtained from the peripheral blood of the individual. In such cells, one can, for example examine the expression profile of one or more genes selected from the group consisting of: ACOT7, ALDH4A1, ATP1B1, ATP2B2, ATP6VOD₁, Clorf54, CD74, CEBPD, CFLAR, CIRBP, CMKOR1, CMTM7, CPNE3, DKFZP56400823, DNAJB1, ELMOD1, EMP3, FAM3C, FAM46A, G1P3, GNA12, HSPB1, ID3, IFITM2, IFITM3, INPPL1, ITGB5, ITPR1, JUN, LOC400566, LY96, LYPD1, MAP2K1, MCL1, MT2A, NEFM, NQ01, P4HA1, PITPNC1, PLEKHC1, PLOD2, PLTP, PREPL, PRKCB1, PTBP1, PTTGIIP, RHBDF2, SERTAD1, SLC29A1, SLC2A5, SOX9, STAMBPL1, TESC, TIMP1, TNFRSFIA, TNPO1, TXNIP, UCHL1, VAMP1, VHL, VIM, ZFP36, ZFP36L1. In some embodiments of the invention, the expression of all genes in this group are examined. In other embodiments of the invention, the expression of one or more of the genes in this group is not examined (e.g. by examining the expression of only 1, 2, 3, 4, 5, 6, 7, 8, 9 or 10 or 20 or 30 or 40 etc. genes in this group). As is known in the art, individuals diagnosed with autism shown dysregulated gene expression in leukocytes (see, e.g. Nishimura et al., Human Molecular Genetics 2007 16(14): 1682-1698; and Hu et al., BMC Genomics 2006, 7: 1-18). Moreover, gene ontology enrichment analysis disclosed in the Example below showed that genes upregulated in autistic cortex were enriched for gene ontology categories implicated in immune and inflammatory response. Genes including those noted immediately above and/or identified in Table B were shown to have overlapping expression patterns with brain and blood cells in one or more data sets, confirming their utility as peripheral biomarkers.

In certain embodiments of the invention, the test cell is obtained from an individual previously identified as exhibiting a behavior associated with autism spectrum disorders. In some embodiments of the invention, the test cell is obtained from an individual identified as having a family member previously identified as exhibiting a behavior associated with autism spectrum disorders. In typical embodiments, the control mammalian cell is obtained from an individual previously identified as not exhibiting a behavior associated with autism spectrum disorders. Embodiments of the invention include methods which perform a further diagnostic procedure for autism spectrum disorders on an individual identified as having a gene expression profile associated with autism spectrum disorders (e.g. a procedure following standard validating measures, such as the Autism Diagnostic Interview (ADI-R)). Optionally, the test mammalian cell and the control mammalian cell are obtained from individuals who are related as siblings or as a parent and a child.

Embodiments of the invention further include a kit comprising: a first container, a label on said container, and a composition contained within said container; wherein the composition includes polymerase chain reaction (PCR) primer effective in the quantitative real time analysis of the mRNA expression levels of one or more DASD genes, the label on said container, or a package insert included in said container indicates that the composition can be used to observe expression levels of one or more DASD genes in at least one type of human leukocyte; a second container comprising a pharmaceutically-acceptable buffer; and instructions for using the PCR primer to obtain an expression profile of the one or more DASD genes. Optionally the kit comprises 2, 3, 4, 5, 6, 7, 8, 9 or 10 polymerase chain reaction (PCR) primers effective in the quantitative real time analysis of the mRNA expression levels of different DASD genes.

In certain embodiments of the invention, a kit further comprises a computer readable a memory storage element adapted to process and store data from one or more expression profiles. In some of these embodiments, the memory storage element organizes expression profile data into a format adapted for electronic comparisons with a library of expression profile data.

As noted above, embodiments of the invention compare DASD gene expression in a test cell (e.g. a cell obtained from an individual suspected of having an autism spectrum disorder) with DASD gene expression in a normal cell (e.g. a cell obtained from an individual not having an autism spectrum disorder) in order to determine if the test cell exhibits altered DASD gene expression. In addition to using normal cells as a comparative sample for DASD expression, in certain situations one can also use a predetermined normative value such as a predetermined normal sequence, and/or level of DASD mRNA or polypeptide expression (see, e.g., Greyer et al., J. Comp. Neurol. 1996 Dec. 9; 376(2):306-14 and U.S. Pat. No. 5,837,501) to evaluate levels of DASD expression in a given sample. The term “status” in this context is used according to its art accepted meaning and refers to the condition or state of a gene and its products. Typically, skilled artisans use a number of parameters to evaluate the condition or state of a gene and its products. These include, but are not limited to the level, sequence of and biological activity of expressed gene products (such as DASD mRNA, polynucleotides and polypeptides). In certain embodiments of the invention, the expression of a DASD gene product is characterized by observing how far the expression level of a DASD mRNA in a sample deviates from a mean expression level of that mRNA in control cells in order to obtain a statistical measure of precision. Standard deviation is a measure of the variability or dispersion of a data set, in this case, the levels of mRNA expression of selected genes. Standard deviation in this context allows determinations of how spread out a set of expression values is and how a given sample fits into such analyses. Illustrative statistical methods for determining such values can be found for example in Cui et al., Genome Biol. (2003) 4:210; Tusher et al., Proc. Natl. Acad. Sci. USA (2001) 98:5116-5121; Jeffery et al., BMC Bioinformatics (2006) 7:359; and Breitling et al., FEBS Lett. (2004) 573:83-92, the contents of which are incorporated by reference.

As discussed in detail below, the status of a DASD gene can be analyzed by a number of techniques that are well known in the art. Typical protocols for evaluating the status of the DASD gene and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). The status of a DASD gene in a biological sample is evaluated by various methods utilized by skilled artisans including, but not limited to single nucleotide polymorphism analyses and genomic Southern analysis (to examine, for example perturbations in DASD genomic sequences), Northern analysis and/or PCR analysis of DASD mRNA (to examine, for example alterations in the polynucleotide sequences or expression levels of DASD mRNAs), and, Western and/or immunohistochemical analysis (to examine, for example alterations in polypeptide sequences, alterations in expression levels of DASD proteins etc.). Detectable DASD polynucleotides include, for example, a DASD gene or fragment thereof, DASD mRNA, alternative splice variants, DASD mRNAs, and recombinant DNA or RNA molecules comprising a DASD polynucleotide.

By examining a biological sample obtained from an individual (e.g. a peripheral blood leukocyte) for evidence altered gene expression of one or more genes whose expression is dysregulated in individuals diagnosed with autism spectrum disorders, medical personnel can obtain information useful in the identification, treatment and/or management of these disorders. Typically, the methods comprise detecting in a sample from a subject the presence of altered DASD gene expression, the presence of the alteration being indicative of the presence of, or predisposition to autism, an autism spectrum disorder, or an autism-associated disorder. In this context, “altered gene expression” encompasses altered DASD mRNA and/or polypeptide levels; altered DASD polynucleotide and polypeptide sequences, altered DASD genomic DNA methylation patterns and the like, alterations that are typically absent in individuals not having an autism spectrum disorder. In such examinations, the status of one or more DASD polynucleotides and/or polypeptides in a biological sample of interest (e.g. a peripheral blood leukocyte obtained from an individual suspected of having an autism spectrum disorder) can be compared to a standard or control, for example, or to the status of the DASD polynucleotide(s) or polypeptide(s) in a corresponding normal sample (e.g. a peripheral blood leukocyte obtained from a non-effected sibling or another individual not having a autism spectrum disorder). An alteration in the status of DASD gene expression in the biological sample (as compared to a control or standardized sample and/or value) then provides evidence of an autism spectrum disorder.

As noted above, embodiments of invention provide methods that comprise for example observing the expression status of one or more DASD genes in a subject in order to obtain diagnostically and/or prognostically useful information. Such methods typically use a leukocyte obtained from a subject to assess the status of a DASD gene. However, the sample may be a variety of biological samples derived from a subject, which contains nucleic acids or polypeptides. Examples of such samples include fluids, tissues, cell samples, organs, biopsies, etc. Most preferred samples are blood and other leukocyte containing tissues etc. Pre-natal diagnosis may also be performed by testing for example fetal cells or placental cells. Other biological samples from which DASD genes and/or the products of DASD genes can be isolated is suitable. The sample may be collected according to conventional techniques and used directly for diagnosis or stored. The sample may be treated prior to performing the method, in order to render or improve availability of nucleic acids and/or polypeptides for testing. Treatments include, for example, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof.

The isolation of biological samples from a subject which contain nucleic acids and/or polypeptides is well know in the art. For example, certain embodiments isolate leukocytes from the circulating blood in order to assess the status of DASD genes in these cells. In such embodiments, blood is typically collected from subjects into heparinized blood collection tubes by personnel trained in phlebotomy using sterile technique. The collected blood samples can be divided into aliquots and centrifuged, and the buffy coat layer can then be removed (this fraction contains the leukocytes). RNA can then be extracted using a commercial RNA purification kit (e.g. RNeasy; Qiagen, Valencia, Calif.). RNA quality can be determined, for example, with an A260/A280 ratio and capillary electrophoresis on an apparatus such as an Agilent 2100 Bioanalyzer automated analysis system (Agilent Technologies, Palo Alto, Calif.).

In typical embodiments of the invention, a sample is contacted with reagents such as probes, primers or ligands (e.g. antibodies) in order to assess the presence of altered gene expression of a DASD gene. Such methods may be performed by a wide variety of apparatuses used in the art, such as a plate, tube, well, glass, etc. In specific embodiments, the contacting is performed on a substrate coated with the reagent, such as a nucleic acid array or a specific ligand (e.g. antibody) array. The substrate may be solid or semi-solid substrate such as any support comprising glass, plastic, nylon, paper, metal, polymers and the like. The substrate may be of various forms and sizes, such as a chip, a slide, a membrane, a bead, a column, a gel, etc. The contacting may be made under any condition suitable for a complex to be formed between the reagent and the nucleic acids or polypeptides of the sample.

A wide variety of methods known in the art can be used to examine the expression of DASD polypeptides and polynucleotides in cells such as peripheral blood leukocytes. For example, certain embodiments of methods which examine DASD polynucleotides and polypeptides in such cells are analogous to those methods from well-established diagnostic assays known in the art such as those that observe the expression of biomarkers such as prostate specific antigen (PSA) polynucleotides and polypeptides. For example, just as PSA polynucleotides are used as probes (for example in Northern analysis, see, e.g., Sharief et al., Biochem. Mol. Biol. Int. 33(3):567-74 (1994)) and primers (for example in PCR analysis, see, e.g., Okegawa et al., J. Urol. 163(4): 1189-1190 (2000)) to observe the presence and/or the level of PSA mRNAs in methods of monitoring PSA expression, the DASD polynucleotides identified herein can be utilized in the same way to observe DASD overexpression or underexpression or other alterations in these genes. Similarly, just as PSA polypeptides are used to generate antibodies specific for PSA which can then be used to observe the presence and/or the level of PSA proteins in methods to monitor PSA protein expression (see, e.g., Stephan et al., Urology 55(4):560-3 (2000)) in prostate cells (see, e.g., Alanen et al., Pathol. Res. Pract. 192(3):233-7 (1996)), the DASD polypeptides described herein can be utilized to generate antibodies for use in detecting DASD expression in peripheral blood leukocytes and the like. Accordingly, the status of DASD gene products provides information useful for predicting a variety of factors including the presence of and/or susceptibility to autism spectrum disorders. As discussed in detail herein, the status of DASD gene products in patient samples can be analyzed by a variety protocols that are well known in the art including immunohistochemical analysis, the variety of Northern blotting techniques including in situ hybridization, RT-PCR analysis (e.g. quantitative RT-PCR), Western blot analysis, polynucleotide and polypeptide microarray analysis and the like.

Exemplary embodiments of the invention include methods for identifying a cell that overexpresses or underexpresses DASD polynucleotides and/or polypeptides. One such embodiment of the invention is an assay that quantifies the expression of the DASD gene in a cell by detecting the absence/presence and/or relative levels of DASD mRNA concentrations in the cell. Methods for the evaluation of particular mRNAs in cells are well known and include, for example, hybridization assays using complementary DNA probes (such as in situ hybridization using labeled DASD riboprobes, Northern blot and related techniques) and various nucleic acid amplification assays (such as qPCR using complementary primers specific for DASD, and other amplification type detection methods, such as, for example, branched DNA, SISBA, TMA and the like).

Embodiments of the invention include methods for detecting a DASD mRNA in a biological sample by generating cDNA in the sample by reverse transcription using at least one primer; amplifying the cDNA so produced using an DASD polynucleotides as sense and antisense primers to amplify DASD cDNAs therein; and detecting the presence of the amplified DASD cDNA. One exemplary PCR method that can be used in embodiments of the invention is a real-time quantitative PCR (qPCR) assay. Such real-time assays provide a large dynamic range of detection and a highly sensitive methods for determining the amount of DNA template of interest. When qPCR follows a reverse transcription reaction, it can be used to quantify RNA templates as well. In addition, qPCR makes quantification of DNA and RNA much more precise and reproducible because it relies on the analysis of PCR kinetics rather than endpoint measurements. Illustrative qPCR assays are disclosed for example in U.S. Patent Application Nos.: 2006/0008809; 2003/0219788; 2006/0051787; and 2006/0099620, the contents of which are incorporated by reference.

Some embodiments of the invention can use next-generation sequencing technologies for the expression profiling of DASD genes, for example those that are commercially available from vendors such as APPLIED BIOSYSTEMS and ILLUMINA (e.g. ILLUMINA Ref8 v3 microarrays). Typically in these embodiments, one can count the number of copies of each DASD gene that is expressed in order to provide assays that quantify the expression levels of all mRNA molecules in a cell. Because such methods are based on sequencing and not hybridization, they can provide an unbiased, probe-less measurement of all mRNA molecules in a sample. Illustrative aspects of such technologies are disclosed for example in U.S. Patent Application Publication No. 20080262747, the contents of which are incorporated by reference.

Another embodiment of the invention is a method of detecting DASD genes having altered copy numbers (i.e. genes having a copy numbers that is above or below the number of copies observed in cells obtained from normal individuals) and/or another chromosomal rearrangement in a biological sample by isolating genomic DNA from the sample; amplifying the isolated genomic DNA using DASD polynucleotides as sense and antisense primers; and detecting the presence of the altered DASD gene. Any number of appropriate sense and antisense probe combinations can be designed from the nucleotide sequence provided for the DASD and used for this purpose.

The invention also provides assays for detecting the presence of a DASD protein in a tissue or other biological sample and the like. Methods for detecting a DASD-related protein are also well known and include, for example, immunoprecipitation, immunohistochemical analysis, Western blot analysis, molecular binding assays, ELISA, ELIFA and the like. For example, a method of detecting the presence of a DASD-related protein in a biological sample comprises first contacting the sample with a DASD antibody, a DASD-reactive fragment thereof, or a recombinant protein containing an antigen binding region of a DASD antibody; and then detecting the binding of DASD-related protein in the sample. Optionally, DASD polypeptide expression is measured in a tissue microarray.

In another embodiment of the invention, one can evaluate the status DASD nucleotide and amino acid sequences in a biological sample in order to identify perturbations in the structure of these molecules. These perturbations can include insertions, deletions, substitutions, duplications and the like in the coding and regulatory regions of the DASD gene. Such evaluations are useful because perturbations in the nucleotide and amino acid sequences are observed in a large number of proteins associated with a growth dysregulated phenotype (see, e.g., Marrogi et al., 1999, J. Cutan. Pathol. 26(8):369-378). For example, a mutation in the sequence of an DASD 5′ or 3′ regulatory enhancer and/or promoter sequence may provide evidence of dysregulated expression. Such assays therefore have diagnostic and predictive value where a mutation in DASD is indicative of dysregulated expression.

A wide variety of assays for observing perturbations in nucleotide and amino acid sequences are well known in the art. For example, the size and structure of nucleic acid or amino acid sequences of DASD gene products are observed by the Northern, Southern, Western, PCR and DNA sequencing protocols discussed herein. In addition, other methods for observing perturbations in nucleotide and amino acid sequences such as single strand conformation polymorphism analysis are well known in the art (see, e.g., U.S. Pat. Nos. 5,382,510 and 5,952,170, the contents of which are incorporated by reference).

The mutation in a DASD gene may be a single base substitution mutation resulting in an amino acid substitution, a single base substitution mutation resulting in a translational stop, an insertion mutation, a deletion mutation, or a gene rearrangement. The mutation may be located in an intron, an exon of the gene, or a promotor or other regulatory region which affects the expression of the gene. Screening for mutated nucleic acids can be accomplished by direct sequencing of nucleic acids. Nucleic acid sequences can be determined through a number of different techniques which are well known to those skilled in the art, for example by chemical or enzymatic methods. The enzymatic methods rely on the ability of DNA polymerase to extend a primer, hybridized to the template to be sequenced, until a chain-terminating nucleotide is incorporated. The most common methods utilize dideoxynucleotides. Primers may be labelled with radioactive or fluorescent labels. Various DNA polymerases are available including Klenow fragment, AMV reverse transcriptase, Thermus aquaticus DNA polymerase, and modified T7 polymerase.

Ligase chain reaction (LCR) is yet another method of screening for mutated nucleic acids. LCR can be carried out in accordance with known techniques and is especially useful to amplify, and thereby detect, single nucleotide differences between two DNA samples. In general, the reaction is carried out with two pairs of oligonucleotide probes: one pair binds to one strand of the sequence to be detected; the other pair binds to the other strand of the sequence to be detected. The reaction is carried out by, first, denaturing (e.g., separating) the strands of the sequence to be detected, then reacting the strands with the two pairs of oligonucleotide probes in the presence of a heat stable ligase so that each pair of oligonucleotide probes hybridize to target DNA and, if there is perfect complementarity at their junction, adjacent probes are ligated together. The hybridized molecules are then separated under denaturation conditions. The process is cyclically repeated until the sequence has been amplified to the desired degree. Detection may then be carried out in a manner like that described above with respect to PCR.

Southern hybridization is also an effective method of identifying differences in sequences. Hybridization conditions, such as salt concentration and temperature can be adjusted for the sequence to be screened. Southern blotting and hybridizations protocols are described in Current Protocols in Molecular Biology (Greene Publishing Associates and Wiley-Interscience), pages 2.9.1-2.9.10. Probes can be labelled for hybridization with random oligomers (primarily 9-mers) and the Klenow fragment of DNA polymerase. Very high specific activity probe can be obtained using commercially available kits such as the Ready-To-Go DNA Labelling Beads (Pharmacia Biotech), following the manufacturer's protocol. Briefly, 25 ng of DNA (probe) is labelled with ³²P-dCTP in a 15 minute incubation at 37° C. Labelled probe is then purified over a ChromaSpin (Clontech) nucleic acid purification column.

Determinations of the presence of the polymorphic form of a DASD protein can also be carried out, for example, by isoelectric focusing, protein sizing, or immunoassay. In an immunoassay, an antibody that selectively binds to the mutated protein can be utilized (for example, an antibody that selectively binds to the mutated form of DASD encoded protein). Such methods for isoelectric focusing and immunoassay are well known in the art. For example, changes resulting in amino acid substitutions, where the substituted amino acid has a different charge than the original amino acid, can be detected by isoelectric focusing. Isoelectric focusing of the polypeptide through a gel having an ampholine gradient at high voltages separates proteins by their pI. The pH gradient gel can be compared to a simultaneously run gel containing the wild-type protein. Protein sizing techniques such as protein electrophoresis and sizing chromatography can also be used to detect changes in the size of the product.

As an alternative to isoelectric focusing or protein sizing, the step of determining the presence of the mutated polypeptides in a sample may be carried out by an antibody assay with an antibody which selectively binds to the mutated polypeptides (i.e., an antibody which binds to the mutated polypeptides but exhibits essentially no binding to the wild-type polypeptide without the polymorphism in the same binding conditions). Antibodies used to selectively bind the products of the mutated genes can be produced by any suitable technique. For example, monoclonal antibodies may be produced in a hybridoma cell line according to the techniques of Kohler and Milstein, Nature, 265, 495 (1975), which is hereby incorporated by reference. A hybridoma is an immortalized cell line which is capable of secreting a specific monoclonal antibody. The mutated products of genes which are associated with autism may be obtained from a human patient, purified, and used as the immunogen for the production of monoclonal or polyclonal antibodies. Purified polypeptides may be produced by recombinant means to express a biologically active isoform, or even an immunogenic fragment thereof may be used as an immunogen. Monoclonal Fab fragments may be produced in Escherichia coli from the known sequences by recombinant techniques known to those skilled in the art.

Additionally, one can examine the methylation status of the DASD gene in a biological sample. Aberrant demethylation and/or hypermethylation of CpG islands in gene 5′ regulatory regions frequently occurs in immortalized and transformed cells, and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al., Am. J. Pathol. 155(6): 1985-1992 (1999)). A variety of assays for examining methylation status of a gene are well known in the art. For example, one can utilize, in Southern hybridization approaches, methylation-sensitive restriction enzymes which cannot cleave sequences that contain methylated CpG sites to assess the methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Unit 12, Frederick M. Ausubel et al. eds., 1995.

Embodiments of the invention include compositions that can be used for example in various methods disclosed herein. Compositions useful in the methods disclosed herein typically include for example one or more DASD nucleic acid molecules designed for use as a probe such as a PCR primer in a method used to monitor DASD mRNAs or genomic sequences in a cell. Optionally, the probe or primer has 8, 9, 19, 11, 12, 13, 14, 15, 16, 17, 18, 19, 20, 21, 22, 23, 24, 25 or more nucleotides that are complementary to a DASD mRNA. In certain embodiments, the probe or primer comprises 5-25 heterologous polynucleotide sequences (e.g. to facilitate cloning). Typically, the probe or primer will hybridize to the DASD mRNA under “stringent conditions” i.e. those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

Specifically contemplated nucleic acid related embodiments of the invention disclosed herein are genomic DNA, cDNAs, ribozymes, and antisense molecules, as well as nucleic acid molecules based on an alternative backbone, or including alternative bases, whether derived from natural sources or synthesized, and include molecules capable of inhibiting the RNA or protein expression of DASD. For example, antisense molecules can be RNAs or other molecules, including peptide nucleic acids (PNAs) or non-nucleic acid molecules such as phosphorothioate derivatives, that specifically bind DNA or RNA in a base pair-dependent manner. Compositions of the invention include one or more antibodies that bind DASD and which can be used as a probe to monitor DASD polypeptide expression in a cell. A skilled artisan can readily prepare these polynucleotide and polypeptide compounds using the DASD polynucleotides and polynucleotide sequences and associated information that is disclosed herein.

For use in the methods described above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for DASD protein or DASD gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kits of the invention have a number of embodiments. A typical embodiment is a kit comprising a container, a label on the container, and a composition contained within the container; wherein the composition includes: (1) a polynucleotide that hybridizes to a complement of the DASD polynucleotide and/or (2) an antibody that binds the DASD polypeptide, the label on the container indicates that the composition can be used to evaluate the expression level of the DASD gene product in at least one type of mammalian cell (e.g. a human peripheral blood leukocyte), and instructions for using the DASD polynucleotide or antibody for evaluating the presence of DASD RNA, DNA or protein in at least one type of mammalian cell.

Autism is a heterogeneous condition and is likely to result from the combined effects of multiple, genetic changes including copy number variations and single nucleotide polymorphisms, interacting with environmental factors (see, e.g. Folstein et al., (2001) Nat. Rev. Genet., 2, 943-955; Belmonte et al., (2004) Mol. Psychiatry, 9, 646-663; Veenstra-Vanderweele et al., (2004) Annu Rev Genomics Hum Genet, 5, 379-405; and Muhle et al., (2004) Pediatrics, 113, e472-486). Classifications such as a computer based hierarchy of autistic patients based on genotypic and phenotypic information is one effective way to identify more homogeneous subgroups and hasten the identification of genes underlying autism (see, e.g. Folstein et al., (2001) Nat. Rev. Genet., 2, 943-955; Belmonte et al., (2004) Mol. Psychiatry, 9, 646-663; Veenstra-Vanderweele et al., (2004) Annu Rev Genomics Hum Genet, 5, 379-405; and Muhle et al., (2004) Pediatrics, 113, e472-486). About 3% of autistic children have either FMR1-FM or dup(15 q), thus comprising more homogeneous populations with a single major genetic etiology for their autism.

In this context, embodiments of the invention further provides methods of obtaining a gene expression profile associated with autism spectrum disorders and methods of generating a database, or collection, of such profiles. The methods generally involve observing a gene expression profile associated with autism spectrum disorders, storing the data on a computer readable medium (CRM), and linking the data with at least one additional data point such as an individual identifying code and/or familial genetic information and/or the presence or absence of other phenomena (e.g. behavioral phenomena) associated with autism spectrum disorders such as Asperger syndrome, pervasive developmental disorder, mental retardation, speech delay, and other associated psychiatric and neurological phenomena. The profile having this information is then recorded on a CRM.

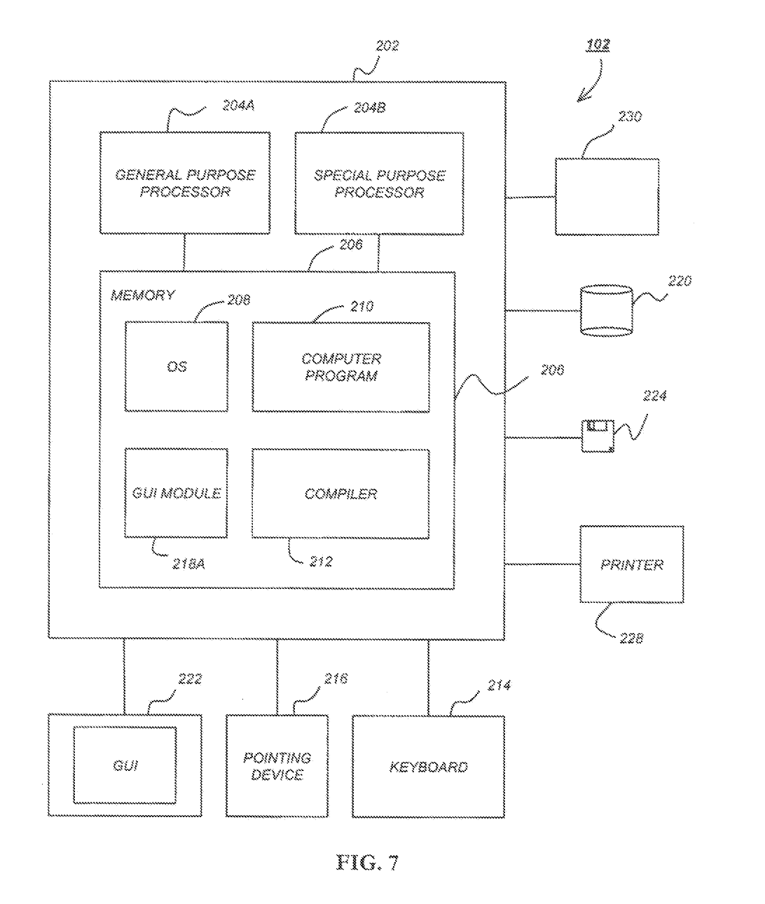

Computer related embodiments of the invention disclosed herein can be performed for example, using one of the many computer systems known in the art. For example, embodiments of the invention can include a searchable database library comprising a plurality of cell profiles recorded on a computer readable medium, each of the profiles comprising further information such as identifying codes and/or familial relationships and/or gene expression and/or behavioral phenomena associated with autism spectrum disorders. In this context, one can then use this library of gene expression and behavioral data to, for example, classify and/or examine etiological subsets of autism as well as to explore the pathophysiology of this condition. In one embodiment of the invention, data obtained from a new test sample is compared to data in such a library in order to, for example, find similar comparative profiles in the library from which diagnostic and/or prognostic information can be inferred. FIG. 7 illustrates an exemplary generalized computer system 202 that can be used to implement elements of the present invention. The computer 202 typically comprises a general purpose hardware processor 204A and/or a special purpose hardware processor 204B (hereinafter alternatively collectively referred to as processor 204) and a memory 206, such as random access memory (RAM). The computer 202 may be coupled to other devices, including input/output (I/O) devices such as a keyboard 214, a mouse device 216 and a printer 228.

In one embodiment, the computer 202 operates by the general purpose processor 204A performing instructions defined by the computer program 210 under control of an operating system 208. The computer program 210 and/or the operating system 208 may be stored in the memory 206 and may interface with the user and/or other devices to accept input and commands and, based on such input and commands and the instructions defined by the computer program 210 and operating system 208 to provide output and results. Output/results may be presented on the display 222 or provided to another device for presentation or further processing or action. In one embodiment, the display 222 comprises a liquid crystal display (LCD) having a plurality of separately addressable liquid crystals. Each liquid crystal of the display 222 changes to an opaque or translucent state to form a part of the image on the display in response to the data or information generated by the processor 204 from the application of the instructions of the computer program 210 and/or operating system 208 to the input and commands. The image may be provided through a graphical user interface (GUI) module 218A. Although the GUI module 218A is depicted as a separate module, the instructions performing the GUI functions can be resident or distributed in the operating system 208, the computer program 210, or implemented with special purpose memory and processors.

Some or all of the operations performed by the computer 202 according to the computer program 210 instructions may be implemented in a special purpose processor 204B. In this embodiment, some or all of the computer program 210 instructions may be implemented via firmware instructions stored in a read only memory (ROM), a programmable read only memory (PROM) or flash memory in within the special purpose processor 204B or in memory 206. The special purpose processor 204B may also be hardwired through circuit design to perform some or all of the operations to implement the present invention. Further, the special purpose processor 204B may be a hybrid processor, which includes dedicated circuitry for performing a subset of functions, and other circuits for performing more general functions such as responding to computer program instructions. In one embodiment, the special purpose processor is an application specific integrated circuit (ASIC).

The computer 202 may also implement a compiler 212 which allows an application program 210 written in a programming language such as COBOL, C++, FORTRAN, or other language to be translated into processor 204 readable code. After completion, the application or computer program 210 accesses and manipulates data accepted from I/O devices and stored in the memory 206 of the computer 202 using the relationships and logic that was generated using the compiler 212. The computer 202 also optionally comprises an external communication device such as a modem, satellite link, Ethernet card, or other device for accepting input from and providing output to other computers.

In one embodiment, instructions implementing the operating system 208, the computer program 210, and the compiler 212 are tangibly embodied in a computer-readable medium, e.g., data storage device 220, which could include one or more fixed or removable data storage devices, such as a zip drive, floppy disc drive 224, hard drive, CD-ROM drive, tape drive, etc. Further, the operating system 208 and the computer program 210 are comprised of computer program instructions which, when accessed, read and executed by the computer 202, causes the computer 202 to perform the steps necessary to implement and/or use the present invention or to load the program of instructions into a memory, thus creating a special purpose data structure causing the computer to operate as a specially programmed computer executing the method steps described herein. Computer program 210 and/or operating instructions may also be tangibly embodied in memory 206 and/or data communications devices 230, thereby making a computer program product or article of manufacture according to the invention. As such, the terms “article of manufacture,” “program storage device,” and “computer program product” as used herein are intended to encompass a computer program accessible from any computer readable device or media.

Of course, those skilled in the art will recognize that any combination of the above components, or any number of different components, peripherals, and other devices, may be used with the computer 202. Although the term “user computer” is referred to herein, it is understood that a user computer 102 may include portable devices such as medication infusion pumps, analyte sensing apparatuses, cellphones, notebook computers, pocket computers, or any other device with suitable processing, communication, and input/output capability.

Embodiments of the invention further comprise, for example, methods of assessing the response of a subject to a treatment of an autism spectrum disorder, or an autism-associated disorder (e.g. treatment comprising the administration of a therapeutic agent), the method comprising detecting altered DASD gene or polypeptide expression (e.g. in multiple genes selected from the group consisting of those whose polynucleotide sequences are shown in SEQ ID NOs: 1-44) in a sample from the treated subject, the presence of the alteration being indicative of a response to the treatment. One embodiment of this invention comprises a method of screening for a compound that modulates DASD mRNA and/or protein expression comprising the steps of contacting a cell that expresses an endogenous or exogenous DASD mRNA and/or protein with one or more compounds and then determining if the one or more compounds modulates DASD mRNA and/or protein expression in the cell (e.g. by qPCR techniques practiced on the cell in the presence and absence of the one or more compounds). Optionally the method comprises observing an effect of a compound on an expression profile of at least one gene comprising a sequence selected from the group consisting of SEQ ID NOs: 1-44, the method comprising the steps of observing an expression profile of the at least one gene in the presence of the compound; and then comparing the expression profile that is observed in the presence of the compound with the expression profile that is observed in the absence of the compound, so that the effect of the compound on an expression profile of the at least one gene is observed.

Another embodiment of this invention comprises a method of screening for a compound that interacts with one or more DASD mRNAs or proteins comprising the steps of contacting one or more compounds with the DASD mRNA and/or protein, and then determining if a compound interacts with the DASD mRNA and/or protein (e.g. by binding techniques that separating compounds that interact with the DASD mRNA and/or protein from compounds that do not). This embodiment of the invention can be used for example to screen chemical libraries for compounds which modulate, e.g., inhibit, antagonize, or agonize or mimic, the expression of a DASD gene as measured by one of the assays disclosed herein. The chemical libraries can be peptide libraries, peptidomimetic libraries, chemically synthesized libraries, recombinant, e.g., phage display libraries, and in vitro translation-based libraries, other non-peptide synthetic organic libraries. Exemplary libraries are commercially available from several sources (e.g. e, Tripos/PanLabs, ChemDesign, Pharmacopoeia). Typical peptide libraries and screening methods that can be used to identify compounds that modulate the expression of and/or interact with DASD protein sequences are disclosed for example in U.S. Pat. Nos. 5,723,286 and 5,733,731, the contents of which are incorporated by reference.

Various aspects of the invention are further described and illustrated by way of the examples that follow, none of which are intended to limit the scope of the invention. Certain disclosure in the examples below can be found in Voineagu et al., Nature 474, 380-384 (2011), the contents of which are incorporated by reference. The disclosure in the Voineagu et al. Nature article is found in U.S. Provisional Application Ser. No. 61/489,471, filed May 24, 2011 (the priority document for the instant application). This Voineagu et al. Nature article provides information that illustrates aspects of the technologies described herein. In addition, certain methods and materials that can be adapted for use with embodiments of the invention can be those found for example in U.S. Patent Application Nos.: 2002/0155450; 2006/0141519; 2007/0134664; and 2009/0011414, the contents of which are incorporated by reference.

### EXAMPLES

### Example 1

### Transcriptomic Analysis of Autistic Brain Reveals Convergent Molecular Pathology

Autism spectrum disorder (ASD) is a common, highly heritable neurodevelopmental condition characterized by marked genetic heterogeneity (see references 1-3, which, like all of the other numerically identified references in this Example, are listed below). Thus, a fundamental question is whether autism represents an etiologically heterogeneous disorder in which the myriad genetic or environmental risk factors perturb common underlying molecular pathways in the brain (4). Here, we demonstrate consistent differences in transcriptome organization between autistic and normal brain by gene co-expression network analysis. Remarkably, regional patterns of gene expression that typically distinguish frontal and temporal cortex are significantly attenuated in the ASD brain, suggesting abnormalities in cortical patterning. We further identify discrete modules of co-expressed genes associated with autism: a neuronal module enriched for known autism susceptibility genes, including the neuronal specific splicing factor A2BP1 (also known as RBFOX1), and a module enriched for immune genes and glial markers. Using high throughput RNA sequencing we demonstrate dysregulated splicing of A2BP1-dependent alternative exons in the ASD brain. Moreover, using a published autism genome-wide association study (GWAS) data set, we show that the neuronal module is enriched for genetically associated variants, providing independent support for the causal involvement of these genes in autism. In contrast, the immune-glial module showed no enrichment for autism GWAS signals, indicating a non-genetic etiology for this process. Collectively, our results provide strong evidence for convergent molecular abnormalities in ASD, and implicate transcriptional and splicing dysregulation as underlying mechanisms of neuronal dysfunction in this disorder.

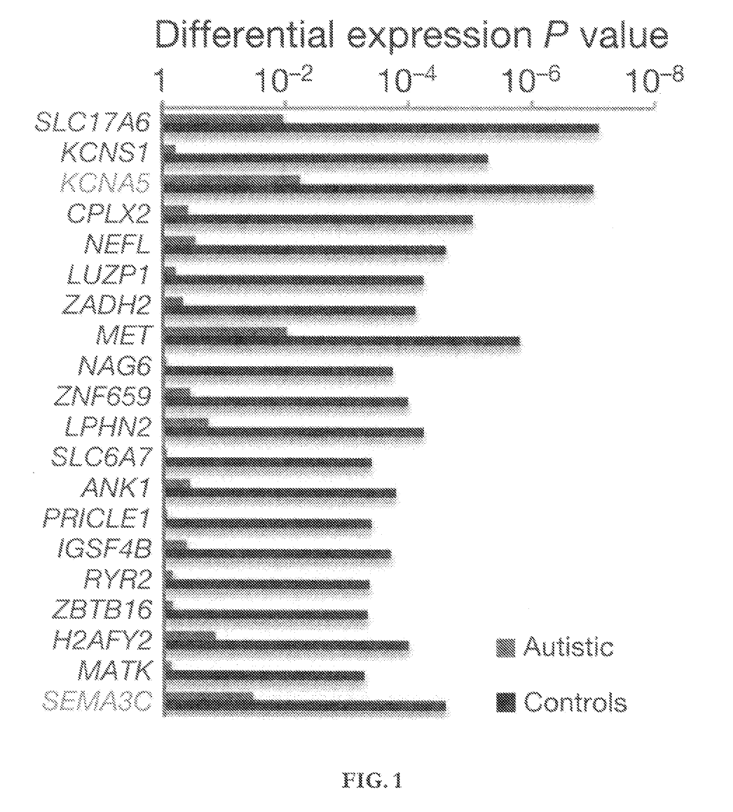

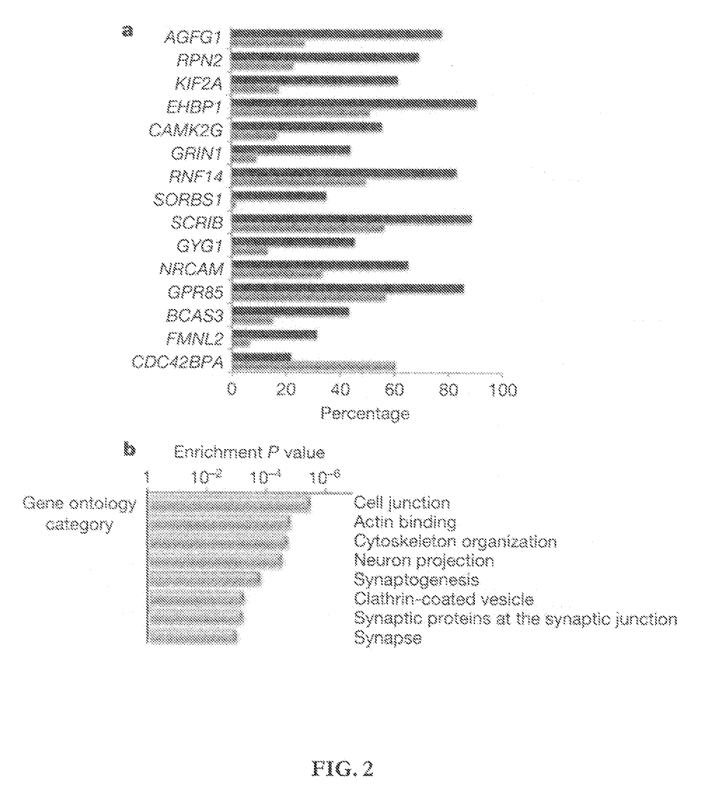

We analyzed post-mortem brain tissue samples from 19 autism cases and 17 controls from the Autism Tissue Project and the Harvard brain bank (see, e.g., supplementary Table 1 in Voineagu et al., Nature 474, 380-384 (2011) using ILLUMINA microarrays. For each individual, we profiled three regions previously implicated in autism (5): superior temporal gyrus (STG, also known as Brodmann's area (BA) 41/42), prefrontal cortex (BA9) and cerebellar vermis. After filtering for high-quality array data (see Methods below), we retained 58 cortex samples (29 autism, 29 controls) and 21 cerebellum samples (11 autism, 10 controls) for further analysis (see Methods for detailed sample description). We identified 444 genes showing significant expression changes in autism cortex samples (DS1, see, e.g., FIG. 1b in Voineagu et al., Nature 474, 380-384 (2011)), and only 2 genes were differentially expressed between the autism and control groups in cerebellum (Methods), indicating that gene expression changes associated with autism were more pronounced in the cerebral cortex, which became the focus of further analysis (see, e.g., supplementary Table 2 in Voineagu et al., Nature 474, 380-384 (2011)). There was no significant difference in age, post mortem interval (PMI), or RNA integrity numbers (RIN) between autism and control cortex samples (see, e.g., supplementary FIG. 1, in Voineagu et al., Nature 474, 380-384 (2011)).

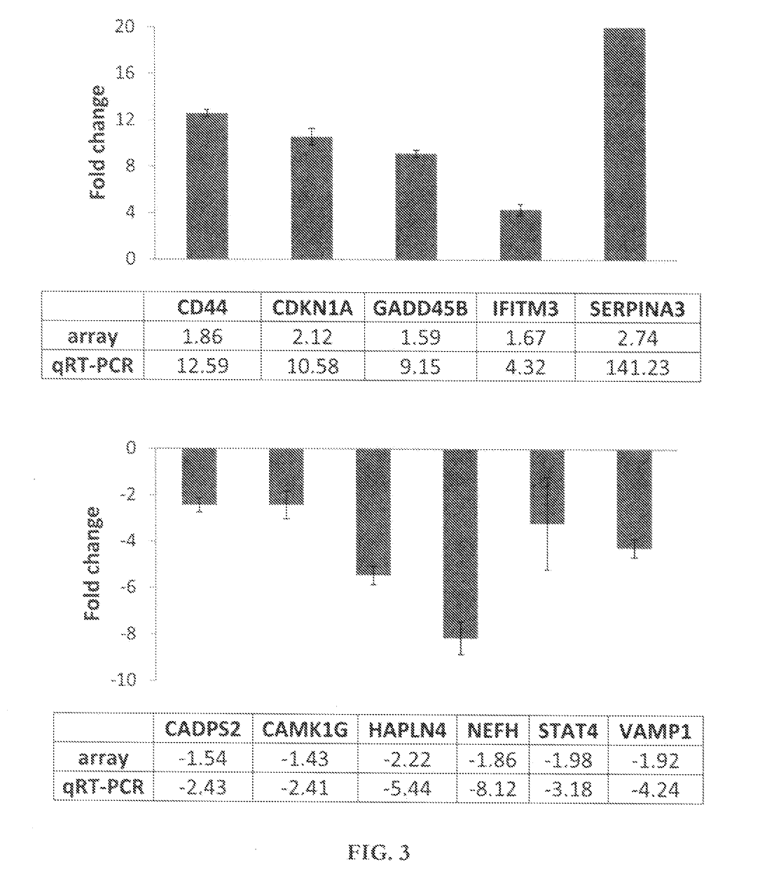

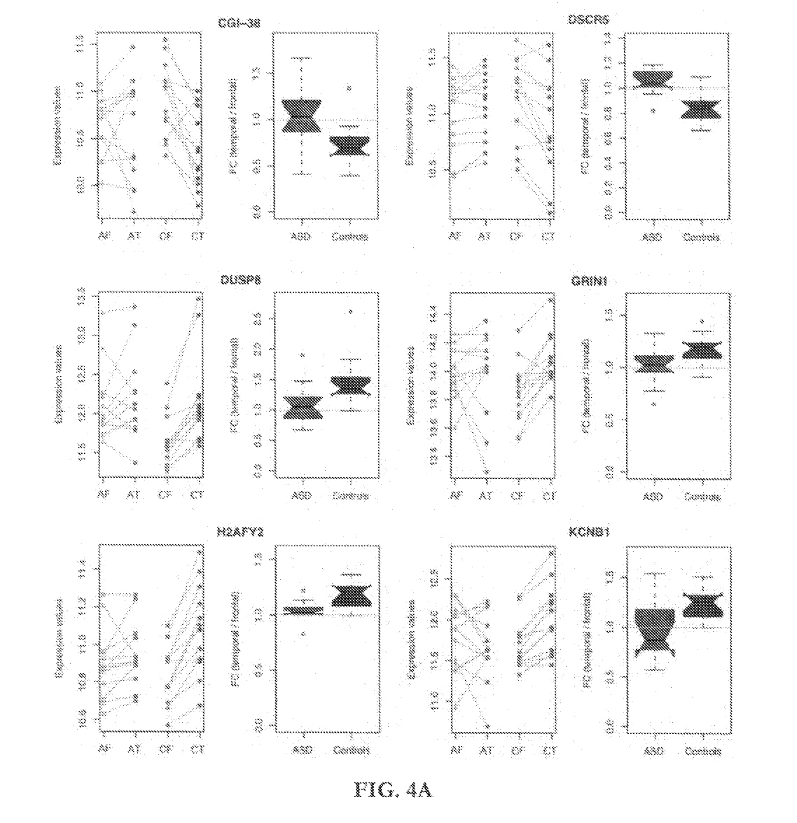

Supervised hierarchical clustering based on the top 200 differentially expressed genes showed distinct clustering of the majority of autism cortex samples (see, e.g., FIG. 1a in Voineagu et al., Nature 474, 380-384 (2011)), including one case that was simultaneously found to have a 15q duplication (see, e.g., supplementary Table 1 in Voineagu et al., Nature 474, 380-384 (2011)), which is known to cause 1% of ASD (6). Cortex samples from ten of the cases coalesced in a single tight-clustering branch of the dendrogram. Clustering was independent of age, sex, RIN, PMI, co-morbidity of seizures, or medication (see, e.g., FIG. 1a and Supplementary FIG. 2c in Voineagu et al., Nature 474, 380-384 (2011)). It is interesting to note that the two ASD cases that cluster with controls (see, e.g., FIG. 1a in Voineagu et al., Nature 474, 380-384 (2011)) are the least severe cases, as assessed by global functioning (see, e.g., supplementary Table 12 in Voineagu et al., Nature 474, 380-384 (2011)). We observed a highly significant overlap between differentially expressed genes in frontal and temporal cortex (P=10⁻⁴⁴; see, e.g., FIG. 1b in Voineagu et al., Nature 474, 380-384 (2011)), supporting the robustness of the data and indicating that the autism-specific expression changes are consistent across these cortical areas. We also validated a cross section of the differentially expressed genes by quantitative reverse transcription PCR (RT-PCR) and confirmed microarray-predicted changes in 83% of the genes tested (see, e.g., FIG. 3 herein and supplementary FIG. 2b in Voineagu et al., Nature 474, 380-384 (2011)). Gene ontology enrichment analysis (Methods) showed that the 209 genes downregulated in autistic cortex were enriched for gene ontology categories related to synaptic function, whereas the upregulated genes (N=235) showed enrichment for gene ontology categories implicated in immune and inflammatory response (see, e.g., supplementary Table 3 in Voineagu et al., Nature 474, 380-384 (2011)).

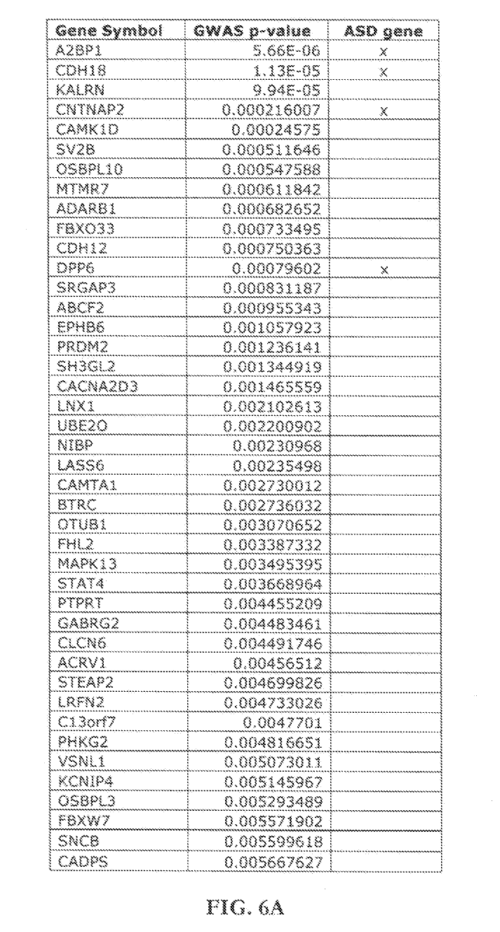

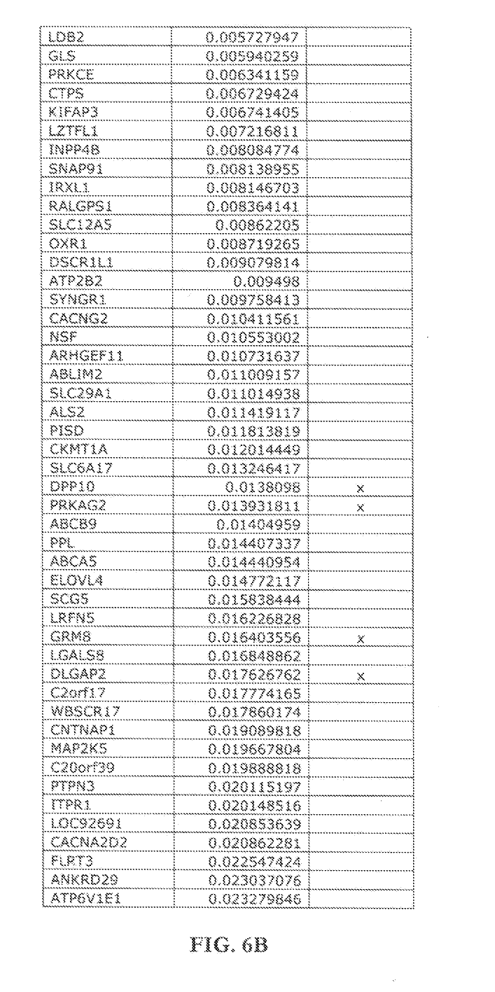

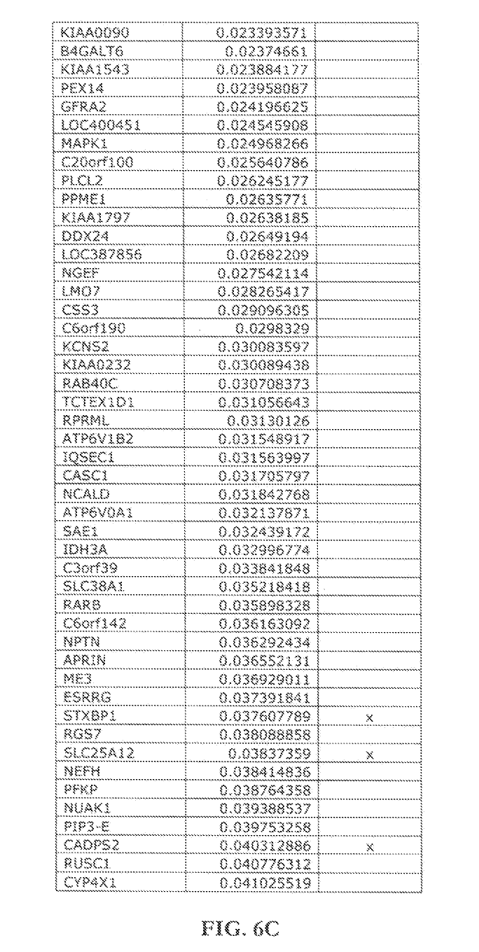

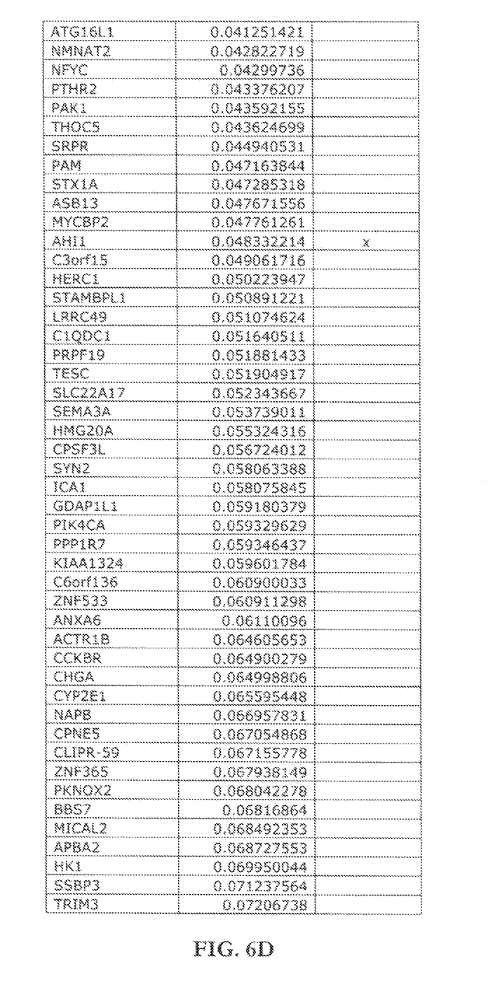

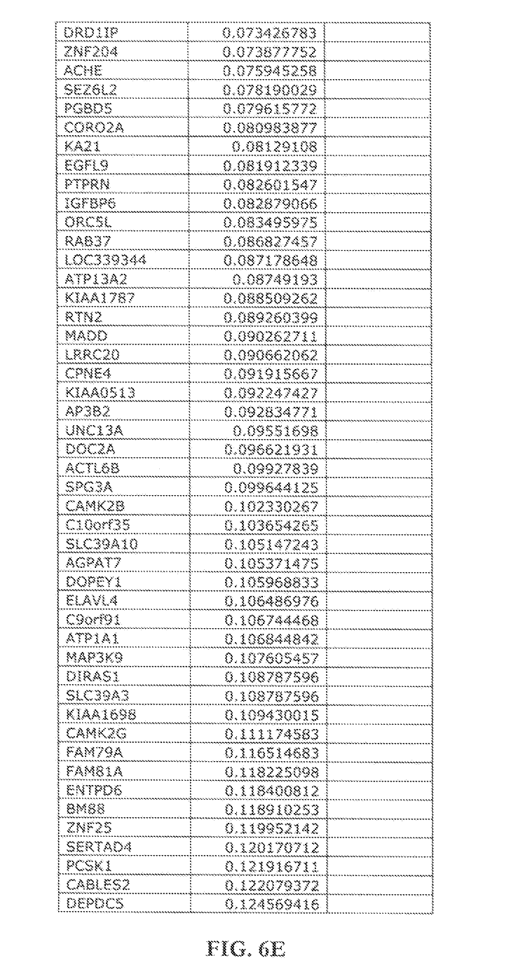

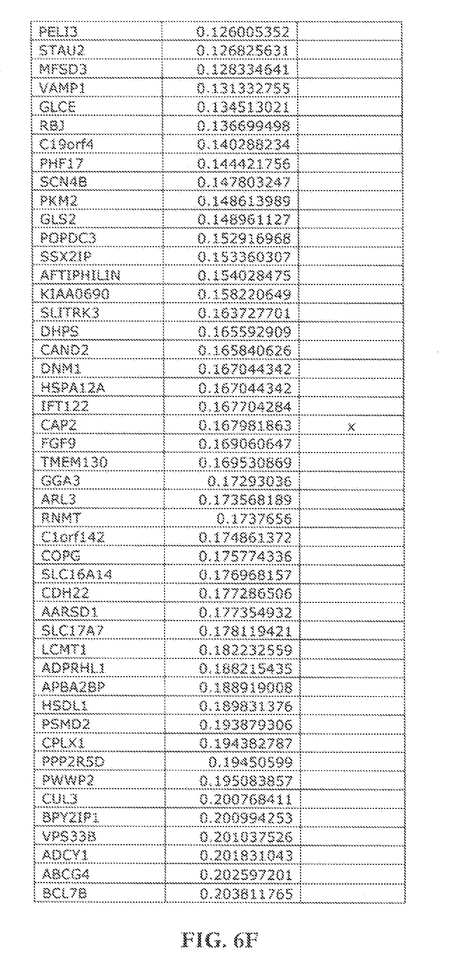

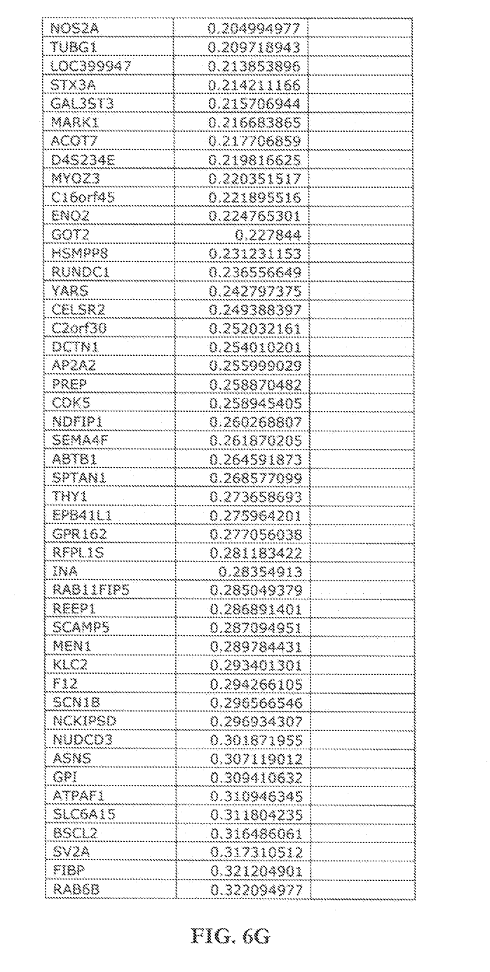

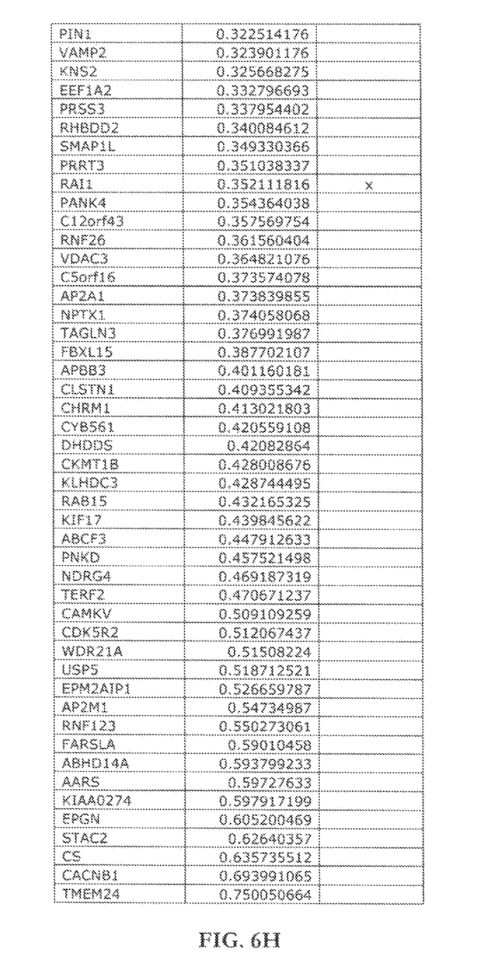

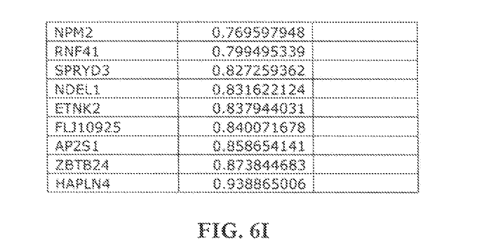

To test whether these findings were replicable, and to further validate the results in an independent data set, we obtained tissue from an additional frontal cortex region (BA44/45) from nine ASD cases and five controls (DS2; see, e.g. supplementary Table 4 in Voineagu et al., Nature 474, 380-384 (2011)). Three of the cases and all of the controls used for validation were independent from our initial cohort. Ninety-seven genes were differentially expressed in BA44/45 in DS2, and 81 of these were also differentially expressed in our initial cohort (P=1.2×10⁻⁹³, hypergeometric test; see, e.g., FIG. 1b, c in Voineagu et al., Nature 474, 380-384 (2011)). Remarkably, the direction of expression differences between autism and controls was the same as in the initial cohort for all but 2 of the 81 overlapping differentially expressed probes. Hierarchical clustering of DS2 samples based on either the top 200 genes differentially expressed in the initial cohort or the 81 overlapping genes showed distinct separation of cases from controls (see, e.g., supplementary FIG. 6 in Voineagu et al., Nature 474, 380-384 (2011)). In addition, comparison of these differentially expressed results with another, smaller study of the STG in ASD (7), revealed significant consistency at the level of differentially expressed genes, including downregulation of DLX1 and AHI1 (see, e.g., supplementary Table 5 in Voineagu et al., Nature 474, 380-384 (2011)). Thus, differential expression analysis produced robust and highly reproducible results, warranting further refined analysis.

We next applied weighted-gene co-expression network analysis (WGCNA) (8,9) to integrate the expression differences observed between autistic and control cerebral cortex into a higher order, systems level context. We first asked whether there are global differences in the organization of the brain transcriptome between autistic and control brain by constructing separate co-expression networks for the autism and control groups (Methods). The control brain network showed high similarity with the previously described human brain co-expression networks (see, e.g., supplementary Table 7 in Voineagu et al., Nature 474, 380-384 (2011)), consistent with the existence of robust modules of co-expressed genes related to specific cell types and biological functions (8). Similarly, the majority of the autism modules (87%) showed significant overlap with the previously described human brain modules (see, e.g., supplementary Table 6 in Voineagu et al., Nature 474, 380-384 (2011)), indicating that many features reflecting the general organization of the autism brain transcriptome are consistent with that of the normal human brain.

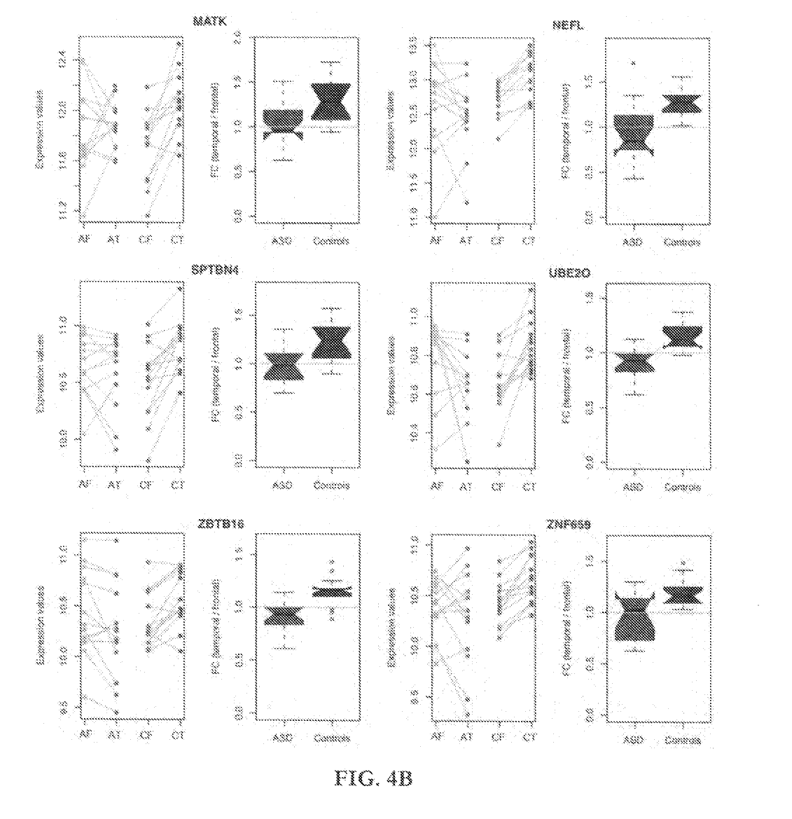

The expression levels of each module were summarized by the first principal component (the module eigengene), and were used to assess whether modules are related to clinical phenotypes or other experimental variables, such as brain region. Two of the control module eigengenes (cM6, cM13) showed significant differences (P<0.05) between the two cortical regions as expected, whereas none of the ASD modules showed any differences between frontal and temporal cortex. This led us to explore the hypothesis that the normal molecular distinctions between the two cortical regions tested were altered in ASD compared with controls. Remarkably, whereas 174 genes were differentially expressed between control BA9 and BA41 (false discovery rate (FDR)<1%), none of the genes were differentially expressed in the same regional comparison among the ASD cases. This was not simply an issue of statistical thresholds, as relaxing the statistical criteria for differential expression to an FDR of 5% identified over 500 differentially expressed genes in controls, and only 8 in ASD brains, confirming the large difference observed in regional cortical differential gene expression between ASD cases and controls (see, e.g., FIG. 1 herein and FIG. 1d, in Voineagu et al., Nature 474, 380-384 (2011)). Analysis of differential expression from a data set (10) of gene expression in developing fetal human brain showed a highly significant (P=5.8×10⁻⁹) overlap of differentially expressed genes with those found in controls in this study, independently confirming that these genes differentiate normal temporal and frontal lobes. We evaluated the homogeneity of gene expression variance across the autism and control groups using Bartlett's test (Methods) which indicated that increased variance was not the major factor responsible for the striking difference in regional gene expression between ASD and controls (see, e.g., FIG. 4 herein and supplementary FIG. 7 and Supplementary Data in Voineagu et al., Nature 474, 380-384 (2011)).

These data suggest that typical regional differences, many of which are observed during fetal development (10), are attenuated in frontal and temporal lobe in autism brain, pointing to abnormal developmental patterning as a potential pathophysiological driver in ASD. This is especially interesting in light of a recent anatomical study of five cases with adult autism which demonstrated a reduction in typical ultra structural differences between three frontal cortical regions in autism (11). Together, these independent studies provide both molecular and structural evidence suggesting a relative diminution of cortical regional identity in autism.

To identify discrete groups of co-expressed genes showing transcriptional differences between autism and controls, we constructed a coexpression network using the entire data set, composed of both autism and control samples (Methods). As previously shown for complex diseases (12,13) co-expression networks allow analysis of gene expression variation related to multiple disease-related and genetic traits. We assessed module eigengene relationship to autism disease status, age, gender, cause of death, co-morbidity of seizures, family history of psychiatric disease, and medication, providing a complementary assessment of these potential confounders to that performed in the standard differential expression analysis (see, e.g., supplementary Table 9 in Voineagu et al., Nature 474, 380-384 (2011)).

The comparison between autism and control groups revealed two network modules whose eigengenes were highly correlated with disease status, and not any of the potential confounding variables (see, e.g., supplementary Table 9 in Voineagu et al., Nature 474, 380-384 (2011)). We found that the top module (M12) showed highly significant enrichment for neuronal markers (see, e.g., supplementary Table 9 in Voineagu et al., Nature 474, 380-384 (2011)), and high overlap with two neuronal modules previously identified as part of the human brain transcriptional network (8): a PVALB+ interneuron module and a module of genes involved in synaptic function. The M12 eigengene was under-expressed in autism cases, indicating that genes in this module were downregulated in the autistic brain (see, e.g., FIG. 2 in Voineagu et al., Nature 474, 380-384 (2011)). Consistent with the pathways identified to be downregulated in autism by differential expression analysis (see, e.g., supplementary Table 3 in Voineagu et al., Nature 474, 380-384 (2011)), the functional enrichment of M12 included the gene ontology categories involved in synaptic function, vesicular transport and neuronal projection.

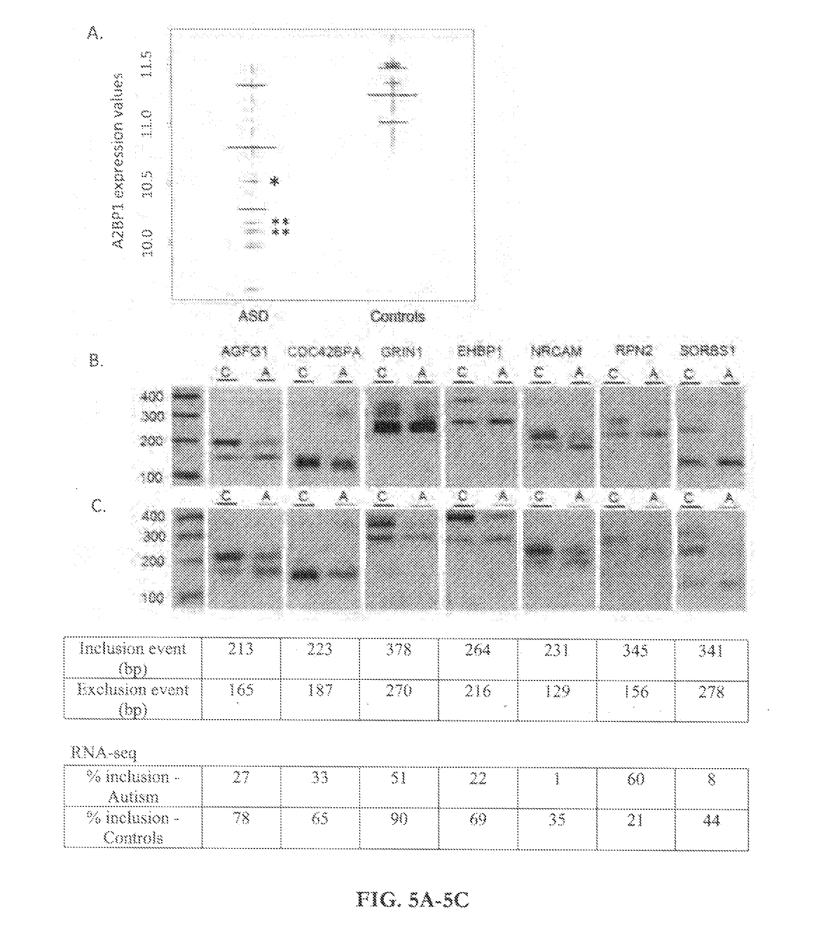

Remarkably, unlike differentially expressed genes, M12 showed significant overrepresentation of known autism susceptibility genes (2) (see, e.g., supplementary Table 10 in Voineagu et al., Nature 474, 380-384 (2011); P=6.1×10⁻⁴), including CADPS2, AHI1, CNTNAP2, and SLC25A12, supporting the increased power of the network-based approach to identify disease-relevant transcriptional changes. A further advantage of network analysis over standard analysis of differential expression is that it allows one to infer the functional relevance of genes based on their network position (9). The hubs of M12, that is, the genes with the highest rank of M12 membership8, were A2BP1, APBA2, SCAMP5, CNTNAP1, KLC2, and CHRM1 (see, e.g., supplementary Data in Voineagu et al., Nature 474, 380-384 (2011)). The first three of these genes have previously been implicated in autism (14-16), whereas the fourth is a homologue of the autism susceptibility gene CNTNAP2 (17). We contemplate the group of genes most strongly connected to the known ASD genes (see, e.g., supplementary FIG. 5 in Voineagu et al., Nature 474, 380-384 (2011)) and emphasize the downregulation of several interneuron markers, such as DLX1 and PVALB, as candidates for future genetic and pathologic investigations.

The second module of co-expressed genes highly related to autism disease status, M16, was enriched for astrocyte markers and markers of activated microglia (see, e.g., supplementary Table 9 in Voineagu et al., Nature 474, 380-384 (2011)), as well as for genes belonging to immune and inflammatory gene ontology categories (see, e.g., FIG. 2 in Voineagu et al., Nature 474, 380-384 (2011)). This module, which was upregulated in ASD brain, showed significant similarity to two modules identified in previous studies of normal human brain (8): an astrocyte module and a microglial module. Consistent with this functional annotation, two of the hubs of the M16 module were known astrocyte markers (ADFP, also known as PLIN2, and IFITM2).

One of the hubs of the M12 module was A2BP1, a neural- and muscle specific alternative splicing regulator (18) and the only splicing factor previously implicated in ASD (16). Because A2BP1 was downregulated in several ASD cases (see, e.g., FIG. 5 herein and supplementary FIG. 8 in Voineagu et al., Nature 474, 380-384 (2011)), this observation provided a unique opportunity to identify potential disease-relevant A2BP1 targets. Whereas A2BP1-regulated alternative exons have been predicted genome-wide (19), few genes have been experimentally validated as A2BP1 targets (20). To identify potential A2BP1-dependent differential splicing events in ASD brain, we performed high-throughput RNA sequencing (RNA-Seq) on three autism samples with significant downregulation of A2BP1 (average fold change by quantitative RT-PCR=5.9) and three control samples with average A2BP1 levels. We identified 212 significant alternative splicing events (see, e.g., supplementary Data in Voineagu et al., Nature 474, 380-384 (2011)). Among these, 36 had been defined (19) as predicted targets of A2BP1/2, which represents a highly significant overlap (P=2.2×10⁻¹⁶). In addition, five previously validated A2BP1 targets showed evidence of alternative splicing, four of which (ATP5C1, ATP2B1, GRIN1 and MEF2C) were confirmed as having differential splicing between ASD samples with low A2BP1 expression and control samples, indicating that we were able to identify a high proportion of the expected A2BP1-dependent differential splicing events. We also observe that alternative exons with increased skipping in ASD relative to control cases are significantly enriched for A2BP1 motifs in adjacent, downstream intronic sequences (P=1.09×10⁻⁷, Fisher's exact test), consistent with previous data (19).

The top gene ontology categories enriched among ASD differential splicing genes highly overlapped with the gene ontology categories found to be enriched in the M12 module (see, e.g., FIG. 2 herein and FIG. 3b in Voineagu et al., Nature 474, 380-384 (2011)). In addition, A2BP1 target genes showed enrichment for actin-binding proteins and genes involved in cytoskeleton reorganization (see, e.g., FIG. 2 herein and FIG. 3b in Voineagu et al., Nature 474, 380-384 (2011)). Among top predicted A2BP1-dependent differential splicing events (see, e.g., FIG. 2 herein and FIG. 3a in Voineagu et al., Nature 474, 380-384 (2011)) are CAMK2G, which also belongs to the M12 module, as well as NRCAM and GRIN1. The latter are proteins involved in synaptogenesis, in which allelic variants have been associated with autism and schizophrenia, respectively (21,22).

RT-PCR assays confirmed a high proportion (85%) of the tested differential splicing changes involving predicted A2BP1 targets (see, e.g., FIG. 5 herein and supplementary FIG. 8 in Voineagu et al., Nature 474, 380-384 (2011)). We further tested the differential splicing events validated by RT-PCR in three independent ASD cases with decreased A2BP1 levels and confirmed the predicted changes in alternative splicing (see, e.g., FIG. 5 herein and supplementary FIG. 8 in Voineagu et al., Nature 474, 380-384 (2011)), indicating that the observed differential splicing events are indeed associated with reduced A2BP1 levels, rather than due to inter-individual variability. The RNA-Seq data thus provides validation of the functional groups of genes identified by coexpression analysis, and evidence for a convergence of transcriptional and alternative-splicing abnormalities in the synaptic and signalling pathogenesis of ASD.

To test whether our findings are more generalizable, and determine whether the autism-associated transcriptional differences observed are likely to be causal, versus collateral effects or environmentally induced changes, we tested whether our co-expression modules or the differentially expressed genes show enrichment for autism genetic association signals. M12 showed highly significant enrichment for association signals (P=5×10⁻⁴), but neither M16 nor the list of differentially expressed genes showed such enrichment (see, e.g., FIG. 4 in Voineagu et al., Nature 474, 380-384 (2011)). As a negative control, we performed the same set-enrichment analysis using two GWAS studies for non-psychiatric disease performed on the same genotyping platform: a genome-wide association for hair colour (23), and a GWAS study of warfarin maintenance dose (24) finding no significant enrichment of the association signal (see, e.g., FIG. 4b, Supplementary FIG. 4 in Voineagu et al., Nature 474, 380-384 (2011)). These results indicate that (1) M12 consists of a set of genes that are supported by independent lines of evidence to be causally involved in ASD pathophysiology, and (2) the upregulation of immune response genes in the autistic brain observed by us and others (25) has no evidence of a common genetic component.

Our system-level analysis of the ASD brain transcriptome demonstrates the existence of convergent molecular abnormalities in ASD for the first time, providing a molecular neuropathological basis for the disease, whose genetic, epigenetic, or environmental etiologies can now be directly explored. The genome-wide analysis performed here significantly extends previous findings implicating synaptic dysfunction, as well as microglial and immune dysregulation in ASD (6) by providing an unbiased systematic assessment of transcriptional alterations and their genetic basis. We show that the transcriptome changes observed in ASD brain converge with GWAS data in supporting the genetic basis of synaptic and neuronal signalling dysfunction in ASD, whereas immune changes have a less pronounced genetic component and thus are most likely either secondary phenomena or caused by environmental factors. Because immune molecules and cells such as microglia have a role in synaptic development and function (26), we speculate that the observed immune upregulation may be related to abnormal ongoing plasticity in the ASD brain. The striking attenuation of gene expression differences observed here between frontal and temporal cortex in ASD is likely to represent a defect of developmental patterning and provides a strong rationale for further studies to assess the pervasiveness of transcriptional patterning abnormalities across the ASD brain. We also demonstrate for the first time alterations in differential splicing associated with A2BP1 levels in the ASD brain, and show that many of the affected exons belong to genes involved in synaptic function. Finally, given current evidence of genetic overlap between ASD and other neurodevelopmental disorders including schizophrenia and attention deficit hyperactivity disorder (ADHD), our data provide a new pathway-based framework from which to assess the enrichment of genetic association signals in other psychiatric disorders.

### Methods

### Brain Tissue.

Post-mortem brain tissue was obtained from the Autism Tissue Project and the Harvard Brain Bank as well as the MRC London Brain bank for Neurodegenerative Disease. Brain tissue samples from 19 autism cases and 17 controls were obtained from the Autism Tissue Project (ATP) and the Harvard Brain Bank. For each brain, tissue was obtained from frontal cortex (BA9), temporal cortex (BA41/42 or BA22) and cerebellum (vermis), with the exception of three controls lacking the cerebellum sample (see, e.g., supplementary Table 1 in Voineagu et al., Nature 474, 380-384 (2011)). For the replication experiment, frontal cortex tissue (BA44/45) from nine ASD cases and five controls were obtained from the ATP and MRCLondon Brain bank for Neurodegenerative Disease respectively (see, e.g., supplementary Table 4 in Voineagu et al., Nature 474, 380-384 (2011)).

For all of the autism cases, clinical information is available upon request from ATP (see www.autismtissueprogram.org), including the ADI-R diagnostic scores. Supplementary Table 12 in Voineagu et al., Nature 474, 380-384 (2011) contains a summary of clinical characteristics. Although autism cases with known genetic causes were not included in this study, one case with a chromosome 15q duplication was identified for AN17138 by high density small nucleotide polymorphism (SNP) arrays (28) during the course of this study. The ATP cases were genotyped with high-density SNP arrays and with two exceptions all are Caucasians. The two Asian samples cluster with the other ASD cases in the current study, and are not distinguishable from the Caucasian cases based on clustering by gene expression.

Microarrays and RNA-Seq.

Total RNA was extracted from 100 mg of tissue using a Qiagen miRNA kit according to the manufacturer's protocol. Expression profiles were obtained using ILLUMINA Ref8 v3 microarrays. RNA-seq was performed on the ILLUMINA GAIIx, as per the manufacturer's instructions. Further detailed information on data analysis is available in Methods. All microarray and RNA-seq data are deposited in GEO under accession number GSE28521.

RNA Extractions and Microarrays.

Total RNA was extracted from approximately 100 mg of frozen tissue, using the Qiagen miRNA kit. RNA concentration was assessed by a NanoDrop and RNA quality was measured using an Agilent Bioanalyzer. All RNA samples included in the expression analysis had an RNA integrity number (RIN)>5. cDNA labelling and hybridizations on ILLUMINA Ref8 v3 microarrays were performed according to the manufacturer's protocol.

Microarray Data Analysis.

Microarray data analysis was performed using the R software and Bioconductor packages. Raw expression data were log 2 transformed and normalized by quantile normalization. Data quality control criteria included high inter-array correlation (Pearson correlation coefficients >0.85) and detection of outlier arrays based on mean inter-array correlation and hierarchical clustering. Probes were considered robustly expressed if the detection P value was <0.05 for at least half of the samples in the data set. Cortex samples (58: 29 autism, 29 controls) and cerebellum samples (21: 11 autism, 10 controls) fulfilled all data quality control criteria. The 29 autism cortex samples included tissue from 13 ASD cases with both frontal and temporal cortex and 3 ASD cases with frontal cortex only (in total 16 frontal cortex and 13 temporal cortex ASD samples). The 29 autism control samples also included tissue from 13 controls with both frontal and temporal cortex and 3 controls with frontal cortex only (in total 16 frontal cortex and 13 temporal cortex control samples).

Initially, all samples were normalized together to assess clustering by brain region. As expected, we observed distinct clustering of cortex and cerebellum samples (see, e.g., supplementary FIG. 2A in Voineagu et al., Nature 474, 380-384 (2011)). For subsequent analyses, cortex samples and cerebellum samples were normalized and analyzed separately.

Differential Expression.

Differential expression was assessed using the SAM package (significance analysis of microarrays, see www-stat.stanford.edu/,tibs/SAM) and unless otherwise specified the significance threshold was FDR<0.05 and fold changes >1.3. Given that SAM is less sensitive in detecting differentially expressed genes for small number of samples, for the replication cohort, the differential expression was assessed by a linear regression method (Limma package, see bioconductor.org/packages/release/bioc/html/limma.html). Our results showing high degree of overlap between genes differentially expressed in the two data sets indicate that the expression differences observed are independent of the analysis methods.

Because 444 genes were differentially expressed between autism and controls in cortex and only 2 genes were differentially expressed between the two groups in cerebellum (FDR<0.05), we tested whether this difference was due to the smaller number of cerebellum samples, by relaxing the statistical criteria to FDR<0.25. We found fewer than 10 differentially expressed genes in cerebellum using the relaxed statistical criteria, supporting the conclusion that genome-wide expression changes in autism were more pronounced in cerebral cortex than in cerebellum.

To account for the fact that the control group of DS1 contained samples from a single female whereas the autism DS1 group included four females, we eliminated from differential expression analysis all probes showing evidence of gender specific gene expression (n=70). We also applied linear regression of expression values against age and sex, and then assessed differential expression between the autism and control groups using the residual values. We observed a 96% overlap between differentially expressed genes using either the residual values or the raw data, indicating that neither age nor sex were major drivers of expression differences between the autism and control groups.

Differential expression between frontal and temporal cortex was assessed by a paired modified t-test (SAM) using the 13 autism and 13 control cases for which RNA samples from both cortex areas passed the quality control criteria. For each of the 510 genes that were differentially expressed in control samples between frontal and temporal cortex, we compared the variance of autism and control expression values in frontal cortex and temporal cortex. The homogeneity of variance (homoscedasticity) of gene expression was assessed using the Barlett test in R. Fifty one genes showed a significant difference in variance (P<0.05, Barlett test) between autism and control groups both in frontal and temporal cortex, and the Barlett test P-values for these genes are listed in Supplementary Data in Voineagu et al., Nature 474, 380-384 (2011).

WGCNA.

Unsigned co-expression networks were built using the WGCNA package in R. Probes with evidence of robust expression (9,914; see above) were included in the network. Network construction was performed using the blockwise Modules function in the WGCNA package (29), which allows the network construction for the entire data set. For each set of genes a pair-wise correlation matrix is computed, and an adjacency matrix is calculated by raising the correlation matrix to a power. The power of 10 was chosen using the scale-free topology criterion (9) and was used for all three networks: the network built using autism samples only, controls samples only or all samples. An advantage of weighted correlation networks is the fact that the results are highly robust with respect to the choice of the power parameter. For each pair of genes, a robust measure of network interconnectedness (topological overlap measure) was calculated based on the adjacency matrix. The topological overlap based dissimilarity was then used as input for average linkage hierarchical clustering. Finally, modules were defined as branches of the resulting clustering tree. To cut the branches, we used the hybrid dynamic tree-cutting because it leads to robustly defined modules (31). To obtain moderately large and distinct modules, we set the minimum module size to 40 genes and the minimum height for merging modules at 0.1. Each module was summarized by the first principal component of the scaled (standardized) module expression profiles. Thus, the module eigengene explains the maximum amount of variation of the module expression levels. For each module, we defined the module membership measure (also known as module eigengene based connectivity kME) as the correlation between gene expression values and the module eigengene. Genes were assigned to a module if they had a high module membership to the module (kME.0.7). An advantage of this definition (and the kME measure) is that it allows genes to be part of more than one module. Genes that did not fulfill these criteria for any of the modules are assigned to the grey module. For the cell type marker enrichment analysis we used the markers defined experimentally in refs (32) and (33) which were previously used to annotate human brain network modules (34,35).

Module visualization: the topological overlap measure was calculated for the top 100 genes in each module ranked by kME. The resulting list of gene pairs was filtered so that both genes in a pair had the highest kME for the module plotted (that is, most module-specific interactions). The resulting top 150 gene pairs were plotted using Visant.

### Gene Ontology Analyses.

Functional enrichment was assessed using the DAVID database see david.abcc.ncifcrf.gov/. For differentially expressed genes and coexpression modules, the background was set to the total list of genes expressed in the brain in the cortex data set. For genes containing differentially spliced exons, the background was set to the total set of genes showing evidence of alternative splicing in our RNA-seq data. The statistical significance threshold level for all gene ontology enrichment analyses was P<0.05 (Benjamin and Hochberg corrected for multiple comparisons).

### Statistical Analyses.

All gene set overlap analyses were performed by assessing cumulative hypergeometric probability using the phyper function in R. The population size was defined as the total number of probes expressed in both data sets. If the comparison involved different platforms, the comparison was done at gene level.

### Quantitative RT-PCR.

One microgram of total RNA was treated with RNase free DNase I (INVITROGEN/Fermentas) and reverse-transcribed using INVITROGEN Superscript II reverse-transcriptase and random hexanucleotide primers (INVITROGEN). Real time PCR was performed on an ABI7900 cycler in 10 ml volume containing iTaq Sybrgreen (BIORAD) and primers at a concentration of 0.5 mM each. The results shown in FIG. 3 (see also supplementary FIG. 2b in Voineagu et al., Nature 474, 380-384 (2011)) represent at least two independent cDNA synthesis experiments for each gene. GAPDH levels were used as an internal control. Statistical significance was assessed by a two-tailed t-test assuming unequal variance.

### Semi-Quantitative RT-PCR.

Total RNA (600 ng) pooled from autism cases (n=2-3) or controls (n=2-3) was reverse-transcribed as described above. cDNA (50 ng) was subjected to 30 cycles of PCR amplification using the primers described in Supplementary Table 11 in Voineagu et al., Nature 474, 380-384 (2011). PCR products were separated on a 3% agarose gel stained with GELSTAR (LONZA).

### RNA Sequencing and Data Analysis.

73-nucleotide reads were generated using an ILLUMINA GAIT sequencer according to the manufacturer's protocol. To generate sufficient read coverage for the quantitative analysis of alternative splicing events, reads for ASD and control brain samples were separately pooled and aligned to an existing database of EST and cDNA-derived alternative splicing junctions using the Basic Local Alignment Tool (BLAT) as described previously (36,37). Reads were considered properly aligned to a splice junction if at least 71 of the 73 nucleotides matched and at least 5 nucleotides mapped to each of the two exons forming the splice junction. Alternative exon inclusion values (“% inc”), representing the proportion of messenger RNA transcripts with the alternatively spliced exon included, were calculated for each mRNA pool as the ratio of reads aligning to the C1-A or A-C2 junctions against reads aligning against all three possible junctions as previously described (36) (C1-A, A-C2, C₁-C₂, see, e.g. supplementary FIG. 3 in Voineagu et al., Nature 474, 380-384 (2011).). Calculated % inc values were considered reliable if at least one of the included junctions as well as the skipped junctions were covered by at least 20 reads. % inc values were compared across samples using Fisher's exact test and the Bonferroni-Hochberg correction to identify differentially spliced exons associated with autism. Differential splicing events were considered significant if they fulfilled both criteria of FDR<0.1 and % inc difference between autism and controls >15%.

### GWAS Set Enrichment Analysis.

GWAS enrichment analysis was performed as previously described in ref. 38 with the main modification that we generated the null distribution, using permutation of gene labels rather than permutation of case/control labels, because the raw genotyping data was not available for all data sets. This approach has been proposed as an acceptable alternative to phenotype label permutation (38) and has been previously used for set enrichment analyses of GWAS data (39). For all genes that met the robust expression criteria in our data set, we mapped the SNPs present on the ILLUMINA 550 k platform located within the transcript boundaries and an additional 20 kb on the 5′ end and 10 kb on the 3′ end. Each gene was assigned a GWAS significance value consisting of the lowest P value of all SNPs mapped to it. A gene set enrichment score (ES) based on the Kolmogorov-Smirnov statistic was calculated as previously described (38) using the 2 log(P-value). The null distribution was generated by 10,000 random permutations of gene labels in the list of genes/P-value pairs and an enrichment score ESp was calculated for each permutation. To correct for the gene set size, the enrichment scores were scaled by subtracting the mean and dividing by the standard deviation of ESp. The resulting z-scores were used to calculate the significance p value.

Primers for q-RT PCR validation of microarray data

| Gene | F/R | Sequence (5′-3′) |
|---|---|---|
| CADPS2 | F | TACCCCTTCAACGCCAAG (SEQ ID NO: 45) |
| CADPS2 | R | CCTGGAACCGTTCTTTCAGT (SEQ ID NO: 46) |
| CAMK1G | F | CTGTAGGAGCTGGAGTGGGA (SEQ ID NO: 47) |
| CAMK1G | R | TTCTTCCTTTCGACCCATTG (SEQ ID NO: 48) |
| CD44 | F | GACAAGTTTTGGTGGCACG (SEQ ID NO: 49) |
| CD44 | R | CACGTGGAATACACCTGCAA (SEQ ID NO: 50) |
| CDKN1A | F | ACCGAGGCACTCAGAGGAG (SEQ ID NO: 51) |
| CDKN1A | R | GCCATTAGCGCATCACAGT (SEQ ID NO: 52) |
| GADD45B | F | ACAGTGGGGGTGTACGAGTC (SEQ ID NO: 53) |
| GADD45B | R | GATGTCATCCTCCTCCTCCTC (SEQ ID NO: 54) |
| HAPLN4 | F | AATGAGCTGGAAGATGACGC (SEQ ID NO: 55) |
| HAPLN4 | R | GAAGGTCAGCTTGTATCGGC (SEQ ID NO: 56) |
| IFITM3 | F | ATGTCGTCTGGTCCCTGTTC (SEQ ID NO: 57) |
| IFITM3 | R | CCAACCATCTTCCTGTCCC (SEQ ID NO: 58) |
| NEFH | F | CAGGACCTGCTCAATGTCAA (SEQ ID NO: 59) |
| NEFH | R | CAAAGCCAATCCGACACTCT (SEQ ID NO: 60) |
| SERPINA3 | F | GGAACTAGGGGGAAAATCACA (SEQ ID NO: 61) |
| SERPINA3 | R | GTCAAAGGGCATCTCCCAT (SEQ ID NO: 62) |
| STAT4 | F | GGTCGTGTTTCCAAAGAGAAA (SEQ ID NO: 63) |
| STAT4 | R | TGCAGCCAATATTCTCCTCTC (SEQ ID NO: 64) |
| VAMP1 | F | CAGCCTCCGGAGAGGAA (SEQ ID NO: 65) |
| VAMP1 | R | CAGTCCCTTCTGTCCCTTCA (SEQ ID NO: 66) |

Primers for semi-quantitative RT PCR validation of differential splicing events

| Gene | | | Inclusion | |
|---|---|---|---|---|
| Exclusion | F/R | Sequence (5′-3′) | event (bp) | event (bp) |
| AGFG1 | F | TCAGACCAATGCCAGAGGAGC (SEQ ID NO: 67) | 213 | 165 |
| AGFG1 | R | GCTGTCTGTTGAGGGAAAGCTG (SEQ ID NO: 68) | 213 | 165 |
| CDC42BPA | F | GTCCTGGAGATGGAATACAGATC (SEQ ID NO: 69) | 342 | 156 |
| CDC42BPA | R | CTGACAAGCCACTGCTAGCAC (SEQ ID NO: 70) | 342 | 156 |
| EHBP1 | F | CAGCAAGATGAAGAGCGACGTC (SEQ ID NO: 71) | 378 | 270 |
| EHBP1 | R | CATGTCCTGCTCTGAGCTCTC (SEQ ID NO: 72) | 378 | 270 |
| GRIN1 | F | AGCATCCACCTGAGCTTCCTG (SEQ ID NO: 73) | 341 | 278 |
| GRIN1 | R | CTGGCAGAAAGGATGATGACCC (SEQ ID NO: 74) | 341 | 278 |
| NRCAM | F | TTCCTGCCAACAAGACACGGTG (SEQ ID NO: 75) | 223 | 187 |
| NRCAM | R | AGACCAATGAACCAGCCCTGAG (SEQ ID NO: 76) | 223 | 187 |
| RPN2 | F | ATGCTGGGACTCATGTATGTCTAC (SEQ ID NO: 77) | 264 | 216 |
| RPN2 | R | CTTCTCATACTGTGAATTGTTCTTGAC (SEQ ID NO: 78) | 264 | 216 |
| SORBS1 | F | GTCGGGATATAAGCCCAGAAGAG (SEQ ID NO: 79) | 231 | 129 |
| SORBS1 | R | CAGGAGTCTCTGAAGAAATTTCCG (SEQ ID NO: 80) | 231 | 129 |

F-forward primer,
R-reverse primer

### REFERENCES

1. Durand, C. M. et al. Nature Genet. 39, 25-27 (2006).
2. Pinto, D. et al. Nature 466, 368-372 (2010).
3. Sebat, J. et al. Science 316, 445-449 (2007).
4. Geschwind, D. H. Cell 135, 391-395 (2008).
5. Amaral et al., Trends Neurosci. 31, 137-145 (2008).
6. Abrahams et al., Nature Rev. Genet. 9, 341-355 (2008).
7. Garbett, K. et al. Neurobiol. Dis. 30, 303-311 (2008).
8. Oldham, M. C. et al. Nature Neurosci. 11, 1271-1282 (2008).
9. Zhang, B. & Horvath, S. (2005) Stat. Appl. Genet. Mol. Biol. 4, 17.
10. Johnson, M. B. et al. Neuron 62, 494-509 (2009).
11. Zikopoulos et al., J. Neurosci. 30, 14595-14609 (2010).
12. Chen, Y. et al. Nature 452, 429-435 (2008).
13. Plaisier, C. L. et al. PLoS Genet. 5, e1000642 (2009).
14. Babatz et al., Autism Res. 2, 359-364 (2009).
15. Castermans, D. et al. Hum. Mol. Genet. 19, 1368-1378 (2010).
16. Martin, C. L. et al. Am. J. Med. Genet. B. Neuropsychiatr. Genet. 144B, 869-876 (2007).
17. Alarcón, M. et al. Am. J. Hum. Genet. 82, 150-159 (2008).
18. Underwood et al., Mol. Cell. Biol. 25, 10005-10016 (2005).
19. Zhang, C. et al. Genes Dev. 22, 2550-2563 (2008).
20. Lee et al., Genes Dev. 23, 2284-2293 (2009).
21. Moy et al., Behay. Brain Res. 205, 123-131 (2009).
22. Zhao, X. et al. Biol. Psychiatry 59, 747-753 (2006).
23. Han, J. et al. PLoS Genet. 4, e1000074 (2008).
24. Cooper, G. M. et al. Blood 112, 1022-1027 (2008).
25. Morgan, J. T. et al. Biol. Psychiatry 68, 368-376 (2010).
26. Boulanger, L. M. Neuron 64, 93-109 (2009).
27. Wang, K. et al. Nature 459, 528-533 (2009).
28. Wintle, R. F. et al. (2010) Autism Res. 4, 89-97 (2011).
29. Langfelder, P. & Horvath, S. WGCNA: an R package for weighted correlation network analysis. BMC Bioinformatics 9, 559 (2008).
31. Langfelder, P., Zhang, B. & Horvath, S. Defining clusters from a hierarchical cluster tree: the Dynamic Tree Cut package for R. Bioinformatics 24, 719-720 (2008).
32. Cahoy, J. D. et al. J. Neurosci. 28, 264-278 (2008).
33. Albright et al. J. Neuroimmunol. 157, 27-38 (2004).
34. Oldham, M. C. et al. Nature Neurosci. 11, 1271-1282 (2008).
35. Miller et al. Proc. Natl. Acad. Sci. USA 107, 12698-12703 (2010).
36. Luco, R. F. et al. Science 327, 996-1000 (2010).
37. Pan et al., Nature Genet. 40, 1413-1415 (2008).
38. Wang et al., Am. J. Hum. Genet. 81, 1278-1283 (2007).
39. Zhang et al., Nucleic Acids Res. 38 (suppl. 2), W90-W95 (2010).

### TABLES

TABLE A
DIFFERENTIAL EXPRESSION IN DASD GENES
Illustrative probes differentially expressed between autism and control
samples are listed below. ILLUMINA Probe ID. FC = fold change.

| Probe ID | Gene Symbol | Cortex_Autism vs Controls_FC |
|---|---|---|
| ILMN_2069224 | PVALB | 0.384281104 |
| ILMN_2165463 | HAPLN4 | 0.450365646 |
| ILMN_1812824 | SST | 0.487002818 |
| ILMN_1801302 | SCN1B | 0.503271564 |
| ILMN_1785202 | STAT4 | 0.519700809 |
| ILMN_1705153 | NEFH | 0.535060416 |
| ILMN_1737611 | VAMP1 | 0.535167674 |
| ILMN_2281786 | RTN1 | 0.548921039 |
| ILMN_1728301 | GAD2 | 0.549928388 |
| ILMN_2292646 | GAD1 | 0.575469672 |
| ILMN_2081813 | PCSK1 | 0.576302013 |
| ILMN_1668035 | CRH | 0.578603948 |
| ILMN_1697512 | SLC32A1 | 0.580940223 |
| ILMN_1748983 | RTN4 | 0.592773439 |
| ILMN_2330845 | NSF | 0.608527579 |
| ILMN_2408080 | SNAP25 | 0.610725452 |
| ILMN_1761903 | KCNS1 | 0.613791146 |
| ILMN_1765966 | CHGB | 0.620605007 |
| ILMN_2413964 | GABRG2 | 0.623626217 |
| ILMN_1757497 | VGF | 0.625709992 |
| ILMN_2071186 | CORT | 0.626115552 |
| ILMN_1772627 | D4S234E | 0.630196216 |
| ILMN_1802633 | GABRA1 | 0.631429459 |
| ILMN_2342554 | TAGLN3 | 0.638467628 |
| ILMN_1672121 | LOC387856 | 0.638807504 |
| ILMN_1779428 | LOC387856 | 0.639435175 |
| ILMN_1684461 | CADPS2 | 0.646152723 |
| ILMN_1674778 | ATP6V1G2 | 0.646571954 |
| ILMN_1788053 | SLC25A12 | 0.646680423 |
| ILMN_2215989 | NEFM | 0.646766037 |
| ILMN_1806473 | NGFRAP1L1 | 0.649374756 |
| ILMN_1763344 | ADCYAP1 | 0.650123932 |
| ILMN_1789166 | SHD | 0.65729442 |
| ILMN_1809613 | NGEF | 0.657683412 |
| ILMN_1704383 | TRIM37 | 0.65772731 |
| ILMN_1713603 | PRKCB1 | 0.658272725 |
| ILMN_1794829 | C6orf117 | 0.65912642 |
| ILMN_2405756 | VAMP1 | 0.659332141 |
| ILMN_1794638 | VIP | 0.660660313 |
| ILMN_1775566 | ATP1A1 | 0.66137894 |
| ILMN_1652540 | C5orf16 | 0.664060869 |
| ILMN_1731062 | NPY | 0.667066471 |
| ILMN_1685834 | AMPH | 0.668795152 |
| ILMN_1758067 | RGS4 | 0.673622398 |
| ILMN_1722559 | NEUROD6 | 0.673944661 |
| ILMN_1735743 | FLJ37440 | 0.676926154 |
| ILMN_1653856 | STS-1 | 0.679758078 |
| ILMN_1718295 | STAC2 | 0.679781142 |
| ILMN_1779241 | CRYM | 0.679937721 |
| ILMN_1673704 | INA | 0.680735329 |
| ILMN_2332250 | ACOT7 | 0.681642287 |
| ILMN_1716019 | RHBDL3 | 0.681797213 |
| ILMN_1707137 | LOC400566 | 0.682741904 |
| ILMN_1716988 | OPN3 | 0.686320687 |
| ILMN_1779343 | SNCB | 0.687477404 |
| ILMN_1774604 | PNKD | 0.687489982 |
| ILMN_2120210 | DSCR1L1 | 0.687632103 |
| ILMN_2165354 | DCAMKL1 | 0.68996275 |
| ILMN_1672094 | DLX1 | 0.691470434 |
| ILMN_1659820 | PTD004 | 0.692654635 |
| ILMN_1809101 | STEAP2 | 0.694583493 |
| ILMN_2080080 | MAP7D2 | 0.695165716 |
| ILMN_1770653 | MAL2 | 0.695318144 |
| ILMN_1662419 | COX7A1 | 0.695872309 |
| ILMN_1804339 | CAMK1G | 0.696983027 |
| ILMN_2255579 | RAB37 | 0.69739304 |
| ILMN_1745817 | NELL1 | 0.697418975 |
| ILMN_1809947 | FLJ30834 | 0.697789248 |
| ILMN_1764780 | SVOP | 0.699929938 |
| ILMN_1756701 | MGC4172 | 0.700484179 |
| ILMN_1788538 | NCALD | 0.700549331 |
| ILMN_1754727 | GPRASP2 | 0.701019441 |
| ILMN_1801703 | CPLX1 | 0.701679843 |
| ILMN_1810604 | ELMOD1 | 0.703235976 |
| ILMN_1731783 | ATP1A1 | 0.703441219 |
| ILMN_1726666 | GPX3 | 0.703449485 |
| ILMN_1679580 | KCNIP4 | 0.703521823 |
| ILMN_1682326 | PCP4 | 0.704164246 |
| ILMN_1685496 | RGS7 | 0.704244753 |
| ILMN_1664071 | TNNT2 | 0.705182859 |
| ILMN_1682799 | STAMBPL1 | 0.705814226 |
| ILMN_1677439 | GLS2 | 0.706002692 |
| ILMN_1721605 | SMYD2 | 0.706030377 |
| ILMN_1803818 | NMNAT2 | 0.706103782 |
| ILMN_1774350 | MYOZ3 | 0.709551452 |
| ILMN_1767365 | PAK1 | 0.710736634 |
| ILMN_1658094 | ZNF365 | 0.712214367 |
| ILMN_2180371 | C12orf24 | 0.713178503 |
| ILMN_2384409 | TAC1 | 0.714342911 |
| ILMN_1716563 | PRKCB1 | 0.714465631 |
| ILMN_1796069 | CBLN2 | 0.714645886 |
| ILMN_1779264 | DSCR2 | 0.71492472 |
| ILMN_1684040 | C6orf190 | 0.715113325 |
| ILMN_1717799 | PRKCE | 0.715200483 |
| ILMN_2365569 | ICA1 | 0.715348345 |
| ILMN_1729075 | PTHR2 | 0.71548016 |
| ILMN_2414878 | STXBP1 | 0.715845667 |
| ILMN_1677746 | CABP1 | 0.716591822 |
| ILMN_1751689 | CHRM1 | 0.717655538 |
| ILMN_1797950 | EXTL2 | 0.718804678 |
| ILMN_1793006 | TAC3 | 0.719187578 |
| ILMN_1690397 | DYNC1I1 | 0.719574163 |
| ILMN_1789505 | ITPR1 | 0.720029469 |
| ILMN_1669410 | CHGA | 0.720484862 |
| ILMN_1654639 | HERC6 | 0.72054581 |
| ILMN_1731612 | UCHL5 | 0.721199072 |
| ILMN_1657855 | ACTL6B | 0.721203267 |
| ILMN_1658499 | SYT13 | 0.721207061 |
| ILMN_1679796 | TOMM20 | 0.722254827 |
| ILMN_1803036 | TARBP1 | 0.722409415 |
| ILMN_1688188 | CADPS | 0.722725629 |
| ILMN_1784783 | NME5 | 0.723678541 |
| ILMN_1723837 | SLC6A17 | 0.723939656 |
| ILMN_1715189 | LHX6 | 0.724004197 |
| ILMN_2344298 | STEAP2 | 0.7244329 |
| ILMN_2357542 | VIP | 0.72454341 |
| ILMN_1699768 | CBLN4 | 0.724750508 |
| ILMN_1808765 | ZNF25 | 0.72506654 |
| ILMN_1656560 | DKFZP564O0823 | 0.72619823 |
| ILMN_2065254 | ELAVL4 | 0.727260104 |
| ILMN_1777261 | FAM3C | 0.727669273 |
| ILMN_2330966 | PTK2B | 0.727707779 |
| ILMN_1729165 | TCEAL6 | 0.729243089 |
| ILMN_2381938 | ATP2B2 | 0.729789904 |
| ILMN_1698726 | SLC25A27 | 0.730023038 |
| ILMN_2042792 | C13orf16 | 0.730431658 |
| ILMN_1778371 | CCBL2 | 0.730979547 |
| ILMN_2359168 | A2BP1 | 0.731078044 |
| ILMN_2082209 | C20orf100 | 0.731282208 |
| ILMN_1713803 | LOC400566 | 0.731732794 |
| ILMN_1668411 | FHL2 | 0.731985286 |
| ILMN_1674661 | CIRBP | 0.732314211 |
| ILMN_2181125 | NAPB | 0.73350304 |
| ILMN_1800270 | GABRG2 | 0.734294209 |
| ILMN_2345319 | PREPL | 0.734922956 |
| ILMN_1758825 | ABLIM2 | 0.735175106 |
| ILMN_1711327 | TRIM37 | 0.735739611 |
| ILMN_1651745 | TMEM25 | 0.735822073 |
| ILMN_1699623 | FAM81A | 0.735837506 |
| ILMN_1786966 | ATRNL1 | 0.735978083 |
| ILMN_1676998 | SCN2B | 0.736444548 |
| ILMN_1656300 | GFRA2 | 0.73647511 |
| ILMN_1656129 | SLC39A10 | 0.736522521 |
| ILMN_2404256 | PTPRT | 0.736687579 |
| ILMN_1659285 | DSCR2 | 0.736975199 |
| ILMN_1685690 | SMCY | 0.737126414 |
| ILMN_1734695 | MAP4 | 0.737278688 |
| ILMN_1669382 | CPLX2 | 0.737419865 |
| ILMN_2210199 | NLGN4Y | 0.738054357 |
| ILMN_1798841 | PLCXD3 | 0.738823623 |
| ILMN_1741156 | ARMCX5 | 0.739252187 |
| ILMN_1720158 | ETS2 | 0.739433777 |
| ILMN_1773307 | NAP1L5 | 0.739497205 |
| ILMN_2056975 | HPRT1 | 0.739677847 |
| ILMN_1814316 | A2BP1 | 0.739897483 |
| ILMN_1665547 | CADPS | 0.740132193 |
| ILMN_1694240 | MAP2K1 | 0.740637561 |
| ILMN_1663092 | CITED2 | 0.741797151 |
| ILMN_1671766 | F12 | 0.744289746 |
| ILMN_1659801 | ATP6V1C1 | 0.744325957 |
| ILMN_1690179 | CRYM | 0.744494171 |
| ILMN_1666904 | SLC17A6 | 0.745337431 |
| ILMN_1805737 | PFKP | 0.745394034 |
| ILMN_1732066 | CKMT1A | 0.745683317 |
| ILMN_1705066 | BTBD11 | 0.745921455 |
| ILMN_1730794 | SERTAD4 | 0.746354594 |
| ILMN_1751569 | C1orf173 | 0.746375475 |
| ILMN_1795826 | ATP6V0D1 | 0.746760234 |
| ILMN_2124241 | MUM1L1 | 0.74676485 |
| ILMN_1728747 | STXBP1 | 0.746770797 |
| ILMN_2288784 | CCDC34 | 0.74710188 |
| ILMN_1754076 | CACNA2D3 | 0.747283189 |
| ILMN_2171295 | PFTK1 | 0.7478083 |
| ILMN_1652244 | POPDC3 | 0.747964426 |
| ILMN_1731507 | A2BP1 | 0.748209685 |
| ILMN_2400292 | MAPK9 | 0.748997044 |
| ILMN_1697418 | RBM9 | 0.749556808 |
| ILMN_2368773 | FAM3C | 0.749756884 |
| ILMN_1807177 | KIAA1797 | 0.749838686 |
| ILMN_1781151 | ARMC8 | 0.749960486 |
| ILMN_1814787 | ICA1 | 0.750939994 |
| ILMN_2044061 | OR2L13 | 0.751897754 |
| ILMN_2075643 | ANKRD29 | 0.752573009 |
| ILMN_1765001 | NAT6 | 0.752975424 |
| ILMN_1669631 | GLRB | 0.753238813 |
| ILMN_1659086 | NEFL | 0.753285609 |
| ILMN_2229379 | KIT | 0.753832901 |
| ILMN_1736015 | PHF17 | 0.75409227 |
| ILMN_1658071 | ATP1B1 | 0.754161622 |
| ILMN_1757387 | UCHL1 | 0.754638858 |
| ILMN_1771652 | BAIAP2L2 | 0.755023826 |
| ILMN_1694799 | PIAS2 | 0.755044426 |
| ILMN_1788886 | TOX | 0.755227223 |
| ILMN_2415073 | ATP2B3 | 0.755531037 |
| ILMN_1750181 | TESC | 0.755901508 |
| ILMN_1666625 | KIF17 | 0.756350784 |
| ILMN_1695947 | SCN4B | 0.756478639 |
| ILMN_1793615 | ME3 | 0.756690571 |
| ILMN_1715384 | B3GNT6 | 0.758204908 |
| ILMN_1803045 | TUBGCP5 | 0.758376695 |
| ILMN_1702296 | CART | 0.758584037 |
| ILMN_2223805 | TSGA14 | 0.758646088 |
| ILMN_1723971 | SLC29A1 | 0.758867247 |
| ILMN_1688452 | LCMT1 | 0.75918473 |
| ILMN_1695003 | PCSK2 | 0.759381713 |
| ILMN_1726743 | MRPS30 | 0.759483809 |
| ILMN_1719998 | C9orf45 | 0.759592351 |
| ILMN_2087941 | ENTPD3 | 0.759680776 |
| ILMN_2218780 | PPM2C | 0.759883578 |
| ILMN_2333319 | PTBP1 | 1.300132976 |
| ILMN_1656368 | ALDH4A1 | 1.300608591 |
| ILMN_2176037 | GNA13 | 1.3014974 |
| ILMN_1733559 | CDC14B | 1.303579166 |
| ILMN_1796734 | SPARC | 1.303906501 |
| ILMN_2376625 | VHL | 1.30431452 |
| ILMN_1758315 | SLC9A9 | 1.304687298 |
| ILMN_1728049 | S100A16 | 1.306483572 |
| ILMN_2286987 | LOC388419 | 1.307115281 |
| ILMN_1771599 | PLOD2 | 1.307729013 |
| ILMN_1693421 | RPN2 | 1.308725149 |
| ILMN_1789830 | CFLAR | 1.309526066 |
| ILMN_2255310 | RPS15A | 1.30991181 |
| ILMN_1815745 | SOX4 | 1.310265138 |
| ILMN_1765446 | EMP3 | 1.310916707 |
| ILMN_1786347 | TNPO1 | 1.312730027 |
| ILMN_1728426 | INPPL1 | 1.31277277 |
| ILMN_1774602 | FBLN2 | 1.313140283 |
| ILMN_1702487 | SGK | 1.313405396 |
| ILMN_1736178 | AEBP1 | 1.315796307 |
| ILMN_1682139 | RAI14 | 1.316216905 |
| ILMN_1703946 | ADORA2B | 1.316279075 |
| ILMN_1727402 | HCLS1 | 1.316576577 |
| ILMN_1714335 | RDH10 | 1.316706415 |
| ILMN_1773389 | PLTP | 1.316991139 |
| ILMN_1690621 | MASS1 | 1.317088722 |
| ILMN_1654966 | SCARA3 | 1.317149318 |
| ILMN_1684391 | PLOD1 | 1.317897837 |
| ILMN_1753342 | SAT | 1.319471258 |
| ILMN_1740466 | FAM46A | 1.31987569 |
| ILMN_1695290 | PLEKHC1 | 1.320032643 |
| ILMN_1668374 | ITGB5 | 1.320222486 |
| ILMN_2177156 | SOX2 | 1.320334654 |
| ILMN_1805466 | SOX9 | 1.320689194 |
| ILMN_1682717 | IER3 | 1.323513955 |
| ILMN_1752075 | MYBPC1 | 1.324220106 |
| ILMN_1668345 | OAF | 1.325429692 |
| ILMN_2176592 | BCHE | 1.325841151 |
| ILMN_2188521 | PVRL3 | 1.325901636 |
| ILMN_1687301 | CSPG2 | 1.327319843 |
| ILMN_1660125 | SFMBT2 | 1.327689835 |
| ILMN_1738854 | CACHD1 | 1.32771151 |
| ILMN_1682034 | HEY2 | 1.327996031 |
| ILMN_1702231 | C1orf54 | 1.328534332 |
| ILMN_1781400 | SLC7A2 | 1.328570512 |
| ILMN_1761247 | PIR | 1.328867166 |
| ILMN_2259119 | PRMT2 | 1.329225118 |
| ILMN_1681679 | TSPO | 1.329660203 |
| ILMN_1803788 | LGALS3 | 1.3298149 |
| ILMN_1719599 | SYTL4 | 1.329919797 |
| ILMN_1675268 | LRP4 | 1.330020664 |
| ILMN_1736567 | CD74 | 1.331116886 |
| ILMN_2130441 | HLA-H | 1.332048268 |
| ILMN_1683133 | KLF15 | 1.332471322 |
| ILMN_1777397 | MSX1 | 1.333305969 |
| ILMN_2197365 | RGS2 | 1.334884967 |
| ILMN_1654289 | ELK1 | 1.334923332 |
| ILMN_2179717 | C9orf61 | 1.336291611 |
| ILMN_1768973 | HIST2H2AC | 1.336334149 |
| ILMN_1774836 | PLOD3 | 1.337460354 |
| ILMN_1796749 | KIF1C | 1.33827213 |
| ILMN_1701603 | ALPL | 1.338442458 |
| ILMN_2184966 | ZHX2 | 1.338828124 |
| ILMN_1753182 | COL20A1 | 1.339243854 |
| ILMN_1795865 | FGFRL1 | 1.339610467 |
| ILMN_1731418 | SP110 | 1.340284786 |
| ILMN_1782439 | CNN3 | 1.340935205 |
| ILMN_1802167 | ALDH1L1 | 1.34107932 |
| ILMN_1709479 | YAP1 | 1.341768801 |
| ILMN_1757338 | PLSCR4 | 1.341989739 |
| ILMN_1698934 | CMTM7 | 1.342479655 |
| ILMN_1804448 | MSI2 | 1.342625697 |
| ILMN_1767523 | IL17RB | 1.343641644 |
| ILMN_1680110 | C10orf116 | 1.34426038 |
| ILMN_1732154 | BCAN | 1.344655146 |
| ILMN_2203950 | HLA-A | 1.345111215 |
| ILMN_2085862 | SLC15A3 | 1.3459625 |
| ILMN_1728197 | CLDN5 | 1.346396612 |
| ILMN_2200659 | SNHG5 | 1.346663975 |
| ILMN_2155719 | NBPF10 | 1.348762329 |
| ILMN_2056032 | CD99 | 1.35087478 |
| ILMN_1798360 | CMKOR1 | 1.351860155 |
| ILMN_2144426 | HIST2H2AA3 | 1.352184014 |
| ILMN_1736670 | PPP1R3C | 1.353817566 |
| ILMN_2216157 | GNA12 | 1.354245467 |
| ILMN_1749213 | SDF2L1 | 1.354720976 |
| ILMN_1781952 | MGST1 | 1.354736305 |
| ILMN_2058251 | VIM | 1.354806838 |
| ILMN_1668863 | LYPD1 | 1.355006983 |
| ILMN_1761280 | NTSR2 | 1.360418039 |
| ILMN_1744897 | KCNN3 | 1.36042729 |
| ILMN_2390853 | CTSH | 1.36272381 |
| ILMN_1723358 | SCARA3 | 1.362824308 |
| ILMN_1675848 | MRCL3 | 1.363338259 |
| ILMN_1773427 | ANKRD15 | 1.363898988 |
| ILMN_1661755 | C9orf88 | 1.363944079 |
| ILMN_2124769 | YBX1 | 1.36765071 |
| ILMN_1705247 | ACSL5 | 1.367657046 |
| ILMN_1723035 | OLR1 | 1.36769625 |
| ILMN_1670638 | PITPNC1 | 1.367892968 |
| ILMN_1673769 | KCNG1 | 1.370111644 |
| ILMN_1750386 | NPPA | 1.370563137 |
| ILMN_1670379 | ANTXR1 | 1.371195234 |
| ILMN_1747683 | AQP4 | 1.371277292 |
| ILMN_1693826 | HAVCR2 | 1.371614522 |
| ILMN_1753312 | PLXDC2 | 1.374163389 |
| ILMN_1773576 | CPNE3 | 1.375129641 |
| ILMN_1712583 | METRN | 1.3780468 |
| ILMN_1732410 | SLC16A9 | 1.380328943 |
| ILMN_1660436 | HSPA1B | 1.381550236 |
| ILMN_2184184 | ANXA1 | 1.381923015 |
| ILMN_1676616 | PTPRZ1 | 1.382212794 |
| ILMN_1751079 | TAP1 | 1.385550053 |
| ILMN_1809477 | CARHSP1 | 1.385652772 |
| ILMN_1680132 | IGSF4 | 1.386411325 |
| ILMN_2205963 | C10orf54 | 1.386894281 |
| ILMN_2352097 | GPR56 | 1.386938495 |
| ILMN_2384122 | GPR56 | 1.387425194 |
| ILMN_2385220 | DFFA | 1.388729352 |
| ILMN_2128750 | PTTG1IP | 1.390959596 |
| ILMN_1701025 | EPHX1 | 1.391766624 |
| ILMN_2227011 | ACSBG1 | 1.394607348 |
| ILMN_1812262 | DDR1 | 1.396131103 |
| ILMN_1775170 | MT1X | 1.398211578 |
| ILMN_1693334 | P4HA1 | 1.399932983 |
| ILMN_1744604 | CYBA | 1.401760599 |
| ILMN_1685194 | CLDN10 | 1.402888402 |
| ILMN_1803988 | MCL1 | 1.403682662 |
| ILMN_1653028 | COL4A1 | 1.404711523 |
| ILMN_1668039 | GYPC | 1.404728614 |
| ILMN_2138589 | MERTK | 1.405217169 |
| ILMN_1760027 | WAS | 1.407637733 |
| ILMN_1670535 | NDRG2 | 1.411625495 |
| ILMN_1709747 | ENDOGL1 | 1.417857251 |
| ILMN_1747019 | PDYN | 1.419964487 |
| ILMN_1724533 | LY96 | 1.422076541 |
| ILMN_2302118 | CCDC50 | 1.422142687 |
| ILMN_1757406 | HIST1H1C | 1.422752303 |
| ILMN_1732296 | ID3 | 1.423123586 |
| ILMN_1719695 | NFKBIZ | 1.423475111 |
| ILMN_2109708 | ECGF1 | 1.425340435 |
| ILMN_1705213 | TMBIM1 | 1.426750314 |
| ILMN_2390919 | FBLN2 | 1.427232232 |
| ILMN_1651496 | HIST1H2BD | 1.427546661 |
| ILMN_1697448 | TXNIP | 1.429543296 |
| ILMN_2175912 | ITGB2 | 1.431048056 |
| ILMN_1772359 | LAPTM5 | 1.434751146 |
| ILMN_1813741 | KCNJ16 | 1.434771222 |
| ILMN_2190084 | VAMP8 | 1.434805821 |
| ILMN_1813639 | C20orf58 | 1.436080804 |
| ILMN_1778977 | TYROBP | 1.43820372 |
| ILMN_2336781 | SOD2 | 1.439605517 |
| ILMN_2322996 | EYA2 | 1.442796588 |
| ILMN_1699856 | RALGDS | 1.445595952 |
| ILMN_2347068 | MKNK2 | 1.445680924 |
| ILMN_1687440 | HIPK2 | 1.446628634 |
| ILMN_1687384 | G1P3 | 1.448875551 |
| ILMN_1806023 | JUN | 1.451374405 |
| ILMN_2086470 | PDGFRA | 1.453070176 |
| ILMN_2132982 | IGFBP5 | 1.458937944 |
| ILMN_1740938 | APOE | 1.459970407 |
| ILMN_2184064 | ARRDC4 | 1.460135156 |
| ILMN_1801246 | IFITM1 | 1.460372479 |
| ILMN_2062468 | IGFBP7 | 1.460457711 |
| ILMN_1685005 | TNFRSF1A | 1.463416696 |
| ILMN_1671337 | SLC2A5 | 1.46404106 |
| ILMN_1766054 | ABCA1 | 1.477720314 |
| ILMN_2172174 | NP | 1.486822375 |
| ILMN_2364022 | SLC16A3 | 1.487026141 |
| ILMN_2394777 | DTNA | 1.489346211 |
| ILMN_1794017 | SERTAD1 | 1.489616024 |
| ILMN_1732071 | HIST2H2BE | 1.489840157 |
| ILMN_1691717 | RHBDF2 | 1.492220423 |
| ILMN_2138765 | ADFP | 1.494781928 |
| ILMN_1723480 | BST2 | 1.496075322 |
| ILMN_1675448 | ZFP36L1 | 1.497557616 |
| ILMN_1659895 | MSN | 1.501647858 |
| ILMN_2090105 | TAGLN2 | 1.502315072 |
| ILMN_1686664 | MT2A | 1.507462376 |
| ILMN_1713124 | AKR1C3 | 1.509948155 |
| ILMN_1718977 | GADD45B | 1.51425696 |
| ILMN_2074860 | RN7SK | 1.516139975 |
| ILMN_1698732 | PALLD | 1.518206478 |
| ILMN_2361603 | NDRG2 | 1.518558116 |
| ILMN_2115490 | NBPF20 | 1.52846846 |
| ILMN_1705750 | TGM2 | 1.537833644 |
| ILMN_1701613 | RARRES3 | 1.538608409 |
| ILMN_1652549 | DTNA | 1.542206535 |
| ILMN_2355168 | MGST1 | 1.543990602 |
| ILMN_1781155 | LYN | 1.547240495 |
| ILMN_1740015 | CD14 | 1.548489789 |
| ILMN_2169152 | SRGN | 1.551518608 |
| ILMN_2409167 | ANXA2 | 1.55748268 |
| ILMN_1662932 | LCP1 | 1.565422786 |
| ILMN_1728478 | CXCL16 | 1.566911305 |
| ILMN_1697176 | GFAP | 1.573654136 |
| ILMN_2165753 | LOC649853 | 1.589360981 |
| ILMN_1707727 | ANGPTL4 | 1.591563923 |
| ILMN_1770338 | TM4SF1 | 1.595979975 |
| ILMN_1651498 | GADD45G | 1.59911241 |
| ILMN_1661599 | DDIT4 | 1.611960674 |
| ILMN_1753143 | RHPN2 | 1.620718361 |
| ILMN_1729188 | HAMP | 1.623494622 |
| ILMN_1801205 | GPNMB | 1.626693272 |
| ILMN_1684982 | PDK4 | 1.627757229 |
| ILMN_1775304 | DNAJB1 | 1.632662357 |
| ILMN_2046730 | S100A10 | 1.63345915 |
| ILMN_1730201 | DTNA | 1.636094723 |
| ILMN_1805750 | IFITM3 | 1.675939595 |
| ILMN_1756982 | CLIC1 | 1.682842296 |
| ILMN_1673352 | IFITM2 | 1.682897581 |
| ILMN_2054297 | PTGS2 | 1.688191961 |
| ILMN_1700183 | AGTRL1 | 1.692387336 |
| ILMN_1789074 | HSPA1A | 1.697058402 |
| ILMN_1803429 | CD44 | 1.700522592 |
| ILMN_1774077 | GBP2 | 1.712235274 |
| ILMN_1782050 | CEBPD | 1.747427977 |
| ILMN_1720829 | ZFP36 | 1.754441848 |
| ILMN_1785902 | C1QC | 1.75467867 |
| ILMN_2302757 | FCGBP | 1.770651662 |
| ILMN_1674236 | HSPB1 | 1.779867804 |
| ILMN_1711566 | TIMP1 | 1.781871241 |
| ILMN_1796409 | C1QB | 1.791729263 |
| ILMN_1735502 | MGC33846 | 1.800927745 |
| ILMN_2396444 | CD14 | 1.830377645 |
| ILMN_1789007 | APOC1 | 1.839643827 |
| ILMN_1708934 | ADM | 1.856402442 |
| ILMN_1782788 | CSDA | 1.874264646 |
| ILMN_1720282 | NQO1 | 1.875783057 |
| ILMN_1801616 | EMP1 | 1.955142358 |
| ILMN_1767556 | C10orf10 | 1.998536768 |
| ILMN_1784602 | CDKN1A | 2.12220785 |
| ILMN_1659766 | BAG3 | 2.177420749 |
| ILMN_1729801 | S100A8 | 2.32258342 |
| ILMN_1788874 | SERPINA3 | 2.745885705 |

TABLE B
ILLUSTRATIVE HUMAN DASD POLYNUCLEOTIDE SEQUENCES
INDENTIFIED USING THE DISCLOSURE PRESENTED HEREIN

ACOT7 Entrez ID: 11332; OMIM: 602587; Uniprot ID: BACH_HUMAN; ENSEMBL
ID: ENSG00000097021
ATGGCGCGGCCCGGGCTCATTCATTCCGCGCCGGGCCTGCCAGACACCTGCGCCCTTCTGCAGCCGCCC
GCCGCATCCGCCGCCGCAGCCCCCAGCATGTCGGGCCCAGACGTCGAGACGCCGTCCGCCATCCAGATC
TGCCGGATCATGCGGCCAGATGATGCCAACGTGGCCGGCAATGTCCACGGGGGGACCATCCTGAAGATG
ATCGAGGAGGCAGGCGCCATCATCAGCACCCGGCATTGCAACAGCCAGAACGGGGAGCGCTGTGTGGCC
GCCCTGGCTCGTGTCGAGCGCACCGACTTCCTGTCTCCCATGTGCATCGGTGAGGTGGCGCATGTCAGC
GCGGAGATCACCTACACCTCCAAGCACTCTGTGGAGGTGCAGGTCAACGTGATGTCCGAAAACATCCTC
ACAGGTGCCAAAAAGCTGACCAATAAGGCCACCCTGTGGTATGTGCCCCTGTCGCTGAAGAATGTGGAC
AAGGTCCTCGAGGTGCCTCCTGTTGTGTATTCCCGGCAGGAGCAGGAGGAGGAGGGCCGGAAGCGGTAT
GAAGCCCAGAAGCTGGAGCGCATGGAGACCAAGTGGAGGAACGGGGACATCGTCCAGCCAGTCCTCAAC
CCAGAGCCGAACACTGTCAGCTACAGCCAGTCCAGCTTGATCCACCTGGTGGGGCCTTCAGACTGCACC
CTGCACGGCTTTGTGCACGGAGGTGTGACCATGAAGCTCATGGATGAGGTCGCCGGGATCGTGGCTGCA
CGCCACTGCAAGACCAACATCGTCACAGCTTCCGTGGACGCCATTAATTTTCATGACAAGATCAGAAAA
GGCTGCGTCATCACCATCTCGGGACGCATGACCTTCACGAGCAATAAGTCCATGGAGATCGAGGTGTTG
GTGGACGCCGACCCTGTTGTGGACAGCTCTCAGAAGCGCTACCGGGCCGCCAGTGCCTTCTTCACCTAC
GTGTCGCTGAGCCAGGAAGGCAGGTCGCTGCCTGTGCCCCAGCTGGTGCCCGAGACCGAGGACGAGAAG
AAGCGCTTTGAGGAAGGCAAAGGGCGGTACCTGCAGATGAAGGCGAAGCGACAGGGCCACGCGGAGCCT
CAGCCCTAG (SEQ ID NO: 1)
ALDH4A1 Entrez ID: 8659; OMIM: 606811; Uniprot ID: AL4A1_HUMAN; ENSEMBL
ID: ENSG00000159423
ATGGAAGCCATCCCATGCGTGGTGGGGGATGAGGAGGTGTGGACGTCGGACGTGCAGTACCAAGTGTCG
CCTTTTAACCATGGACATAAGGTGGCCAAGTTCTGTTATGCAGACAAGAGCCTGCTCAACAAAGCCATT
GAGGCTGCCCTGGCTGCCCGGAAAGAGTGGGACCTGAAGCCTATTGCAGACCGGGCCCAGATCTTCCTG
AAGGCGGCAGACATGCTGAGTGGGCCGCGCAGGGCTGAGATCCTCGCCAAGACCATGGTGGGACAGGGT
AAGACCGTGATCCAAGCGGAGATTGACGCTGCAGCGGAACTCATCGACTTCTTCCGGTTCAATGCCAAG
TATGCGGTGGAGCTGGAGGGGCAGCAGCCCATCAGCGTGCCCCCGAGCACCAACAGCACGGTGTACCGG
GGTCTGGAGGGCTTCGTGGCGGCCATCTCGCCCTTTAACTTCACTGCAATCGGCGGCAACCTGGCGGGG
GCACCGGCCCTGATGGGCAACGTGGTCCTATGGAAGCCCAGTGACACTGCCATGCTGGCCAGCTATGCT
GTCTACCGCATCCTTCGGGAGGCTGGCCTGCCCCCCAACATCATCCAGTTTGTGCCAGCTGATGGGCCC
CTATTTGGGGACACTGTCACCAGCTCAGAGCACCTCTGTGGCATCAACTTCACAGGCAGTGTGCCCACC
TTCAAACACCTGTGGAAGCAGGTGGCCCAGAACCTGGACCGGTTCCACACCTTCCCACGCCTGGCTGGA
GAGTGCGGCGGAAAGAACTTCCACTTCGTGCACCGCTCGGCCGACGTGGAGAGCGTGGTGAGCGGGACC
CTCCGCTCAGCCTTCGAGTACGGTGGCCAGAAGTGTTCCGCGTGCTCGCGTCTCTACGTGCCGCACTCG
CTGTGGCCGCAGATCAAAGGGCGGCTGCTGGAGGAGCACAGTCGGATCAAAGTGGGCGACCCTGCAGAG
GATTTTGGGACCTTCTTCTCTGCAGTGATTGATGCCAAGTCCTTTGCCCGTATCAAGAAGTGGCTGGAG
CACGCACGCTCCTCACCCAGCCTCACCATCCTGGCCGGGGGCAAGTGTGATGACTCCGTGGGCTACTTT
GTGGAGCCCTGCATCGTGGAGAGCAAGGACCCTCAGGAGCCCATCATGAAGGAGGAGATCTTCGGGCCT
GTACTGTCTGTGTACGTCTACCCGGATGACAAGTACAAGGAGACGCTGCAGCTGGTTGACAGCACCACC
AGCTATGGCCTCACGGGGGCAGTGTTCTCCCAGGATAAGGACGTCGTGCAGGAGGCCACAAAGGTGCTG
AGGAATGCTGCCGGCAACTTCTACATCAACGACAAGTCCACTGGCTCGATAGTGGGCCAGCAGCCCTTT
GGGGGGGCCCGAGCCTCTGGAACCAATGACAAGCCAGGGGGCCCACACTACATCCTGCGCTGGACGTCG
CCGCAGGTCATCAAGGAGACACATAAGCCCCTGGGGGACTGGAGCTACGCGTACATGCAGTGA (SEQ
ID NO: 2)
ATP1B1 Entrez ID: 8659; OMIM: 606811; Uniprot ID: AL4A1_HUMAN; ENSEMBL
ID: ENSG00000159423
ATGGCCCGCGGGAAAGCCAAGGAGGAGGGCAGCTGGAAGAAATTCATCTGGAACTCAGAGAAGAAGGAG
TTTCTGGGCAGGACCGGTGGCAGTTGGTTTAAGATCCTTCTATTCTACGTAATATTTTATGGCTGCCTG
GCTGGCATCTTCATCGGAACCATCCAAGTGATGCTGCTCACCATCAGTGAATTTAAGCCCACATATCAG
GACCGAGTGGCCCCGCCAGGATTAACACAGATTCCTCAGATCCAGAAGACTGAAATTTCCTTTCGTCCT
AATGATCCCAAGAGCTATGAGGCATATGTACTGAACATAGTTAGGTTCCTGGAAAAGTACAAAGATTCA
GCCCAGAGGGATGACATGATTTTTGAAGATTGTGGCGATGTGCCCAGTGAACCGAAAGAACGAGGAGAC
TTTAATCATGAACGAGGAGAGCGAAAGGTCTGCAGATTCAAGCTTGAATGGCTGGGAAATTGCTCTGGA
TTAAATGATGAAACTTATGGCTACAAAGAGGGCAAACCGTGCATTATTATAAAGCTCAACCGAGTTCTA
GGCTTCAAACCTAAGCCTCCCAAGAATGAGTCCTTGGAGACTTACCCAGTGATGAAGTATAACCCAAAT
GTCCTTCCCGTTCAGTGCACTGGCAAGCGAGATGAAGATAAGGATAAAGTTGGAAATGTGGAGTATTTT
GGACTGGGCAACTCCCCTGGTTTTCCTCTGCAGTATTATCCGTACTATGGCAAACTCCTGCAGCCCAAA
TACCTGCAGCCCCTGCTGGCCGTACAGTTCACCAATCTTACCATGGACACTGAAATTCGCATAGAGTGT
AAGGCGTACGGTGAGAACATTGGGTACAGTGAGAAAGACCGTTTTCAGGGACGTTTTGATGTAAAAATT
GAAGTTAAGAGCTGA (SEQ ID NO: 3)
ATP6V0D1 Entrez ID: 9114; OMIM: 607028; Uniprot ID: VA0D1_HUMAN;
ENSEMBL ID: ENSG00000159720
ATGTCGTTCTTCCCGGAGCTTTACTTTAACGTGGACAATGGCTACTTGGAGGGACTGGTGCGCGGCCTG
AAGGCCGGGGTGCTCAGCCAGGCCGACTACCTCAACCTGGTGCAGTGCGAGACGCTAGAGGACTTGAAA
CTGCATCTGCAGAGCACTGATTATGGTAACTTCCTGGCCAACGAGGCATCACCTCTGACGGTGTCAGTC
ATCGATGACCGGCTCAAGGAGAAGATGGTGGTGGAGTTCCGCCACATGAGGAACCATGCCTATGAGCCA
CTCGCCAGCTTCCTAGACTTCATTACTTACAGTTACATGATCGACAACGTGATCCTGCTCATCACAGGC
ACGCTGCACCAGCGCTCCATCGCTGAGCTCGTGCCCAAGTGCCACCCACTAGGCAGCTTCGAGCAGATG
GAGGCCGTGAACATTGCTCAGACACCTGCTGAGCTCTACAATGCCATTCTGGTGGACACGCCTCTTGCG
GCTTTTTTCCAGGACTGCATTTCAGAGCAGGACCTTGACGAGATGAACATCGAGATCATCCGCAACACC
CTCTACAAGGCCTACCTGGAGTCCTTCTACAAGTTCTGCACCCTACTGGGCGGGACTACGGCTGATGCC
ATGTGCCCCATCCTGGAGTTTGAAGCAGACCGCCGCGCCTTCATCATCACCATCAATTCTTTCGGCACA
GAGCTGTCCAAAGAGGACCGTGCCAAGCTCTTTCCACACTGTGGGCGGCTCTACCCTGAGGGCCTGGCG
CAGCTGGCTCGGGCTGACGACTATGAACAGGTCAAGAACGTGGCCGATTACTACCCGGAGTACAAGCTG
CTCTTCGAGGGTGCAGGTAGCAACCCTGGAGACAAGACGCTGGAGGACCGATTCTTTGAGCACGAGGTA
AAGCTGAACAAGTTGGCCTTCCTGAACCAGTTCCACTTTGGTGTCTTCTATGCCTTCGTGAAGCTCAAG
GAGCAGGAGTGTCGCAACATCGTGTGGATCGCTGAATGTATCGCCCAGCGCCACCGCGCCAAAATCGAC
AACTACATCCCTATCTTCTAG (SEQ ID NO: 4)
C1orf54 Entrez ID: 79630; OMIM:; Uniprot ID: CA054_HUMAN; ENSEMBL ID:
ENSG00000118292
ATGGATGTCCTCTTTGTAGCCATCTTTGCTGTGCCACTTATCCTGGGACAAGAATATGAGGATGAAGAA
AGACTGGGAGAGGATGAATATTATCAGGTGGTCTATTATTATACAGTCACCCCCAGTTATGATGACTTT
AGTGCAGATTTCACCATTGATTACTCCATATTTGAGTCAGAGGACAGGCTGAACAGGTTGGATAAGGAC
ATAACAGAAGCAATAGAGACTACCATTAGTCTTGAAACAGCACGTGCAGACCATCCGAAGCCTGTAACT
GTGAAACCAGTAACAACGGAACCTAGTCCAGATCTGAACGATGCCGTGTCCAGTTTGCGAAGTCCTATT
CCCCTCCTCCTGTCGTGTGCCTTTGTTCAGGTGGGGATGTATTTCATGTAG (SEQ ID NO: 5)
CD74 Entrez ID: 972; OMIM: 142790; Uniprot ID: HG2A_HUMAN; ENSEMBL ID:
ENSG00000019582
ATGCACAGGAGGAGAAGCAGGAGCTGTCGGGAAGATCAGAAGCCAGTCATGGATGACCAGCGCGACCTT
ATCTCCAACAATGAGCAACTGCCCATGCTGGGCCGGCGCCCTGGGGCCCCGGAGAGCAAGTGCAGCCGC
GGAGCCCTGTACACAGGCTTTTCCATCCTGGTGACTCTGCTCCTCGCTGGCCAGGCCACCACCGCCTAC
TTCCTGTACCAGCAGCAGGGCCGGCTGGACAAACTGACAGTCACCTCCCAGAACCTGCAGCTGGAGAAC
CTGCGCATGAAGCTTCCCAAGCCTCCCAAGCCTGTGAGCAAGATGCGCATGGCCACCCCGCTGCTGATG
CAGGCGCTGCCCATGGGAGCCCTGCCCCAGGGGCCCATGCAGAATGCCACCAAGTATGGCAACATGACA
GAGGACCATGTGATGCACCTGCTCCAGAGTCACTGGAACTGGAGGACCCGTCTTCTGGGCTGGGTGTGA
(SEQ ID NO: 6)
CEBPD Entrez ID: 1052; OMIM: 116898; Uniprot ID: CEBPD_HUMAN; ENSEMBL
ID: ENSG000002218693
ATGAGCGCCGCGCTCTTCAGCCTGGACGGCCCGGCGCGCGGCGCGCCCTGGCCTGCGGAGCCTGCGCCC
TTCTACGAACCGGGCCGGGCGGGCAAGCCGGGCCGCGGGGCCGAGCCAGGGGCCCTAGGCGAGCCAGGC
GCCGCCGCCCCCGCCATGTACGACGACGAGAGCGCCATCGACTTCAGCGCCTACATCGACTCCATGGCC
GCCGTGCCCACCCTGGAGCTGTGCCACGACGAGCTCTTCGCCGACCTCTTCAACAGCAATCACAAGGCG
GGCGGCGCGGGGCCCCTGGAGCTTCTTCCCGGCGGCCCCGCGCGCCCCTTGGGCCCGGGCCCTGCCGCT
CCCCGCCTGCTCAAGCGCGAGCCCGACTGGGGCGACGGCGACGCGCCCGGCTCGCTGTTGCCCGCGCAG
GTGGCCGCGTGCGCACAGACCGTGGTGAGCTTGGCGGCCGCAGGGCAGCCCACCCCGCCCACGTCGCCG
GAGCCGCCGCGCAGCAGCCCCAGGCAGACCCCCGCGCCCGGCCCCGCCCGGGAGAAGAGCGCCGGCAAG
AGGGGCCCGGACCGCGGCAGCCCCGAGTACCGGCAGCGGCGCGAGCGCAACAACATCGCCGTGCGCAAG
AGCCGCGACAAGGCCAAGCGGCGCAACCAGGAGATGCAGCAGAAGTTGGTGGAGCTGTCGGCTGAGAAC
GAGAAGCTGCACCAGCGCGTGGAGCAGCTCACGCGGGACCTGGCCGGCCTCCGGCAGTTCTTCAAGCAG
CTGCCCAGCCCGCCCTTCCTGCCGGCCGCCGGGACAGCAGACTGCCGGTAA (SEQ ID NO: 7)
CFLAR Entrez ID: 8837; OMIM: 603599; Uniprot ID: CFLAR_HUMAN; ENSEMBL
ID: ENSG00000003402
ATGTCTGCTGAAGTCATCCATCAGGTTGAAGAAGCACTTGATACAGATGAGAAGGAGATGCTGCTCTTT
TTGTGCCGGGATGTTGCTATAGATGTGGTTCCACCTAATGTCAGGGACCTTCTGGATATTTTACGGGAA
AGAGGTAAGCTGTCTGTCGGGGACTTGGCTGAACTGCTCTACAGAGTGAGGCGATTTGACCTGCTCAAA
CGTATCTTGAAGATGGACAGAAAAGCTGTGGAGACCCACCTGCTCAGGAACCCTCACCTTGTTTCGGAC
TATAGAGTGCTGATGGCAGAGATTGGTGAGGATTTGGATAAATCTGATGTGTCCTCATTAATTTTCCTC
ATGAAGGATTACATGGGCCGAGGCAAGATAAGCAAGGAGAAGAGTTTCTTGGACCTTGTGGTTGAGTTG
GAGAAACTAAATCTGGTTGCCCCAGATCAACTGGATTTATTAGAAAAATGCCTAAAGAACATCCACAGA
ATAGACCTGAAGACAAAAATCCAGAAGTACAAGCAGTCTGTTCAAGGAGCAGGGACAAGTTACAGGAAT
GTTCTCCAAGCAGCAATCCAAAAGAGTCTCAAGGATCCTTCAAATAACTTCAGGCTCCATAATGGGAGA
AGTAAAGAACAAAGACTTAAGGAACAGCTTGGCGCTCAACAAGAACCAGTGAAGAAATCCATTCAGGAA
TCAGAAGCTTTTTTGCCTCAGAGCATACCTGAAGAGAGATACAAGATGAAGAGCAAGCCCCTAGGAATC
TGCCTGATAATCGATTGCATTGGCAATGAGACAGAGCTTCTTCGAGACACCTTCACTTCCCTGGGCTAT
GAAGTCCAGAAATTCTTGCATCTCAGTATGCATGGTATATCCCAGATTCTTGGCCAATTTGCCTGTATG
CCCGAGCACCGAGACTACGACAGCTTTGTGTGTGTCCTGGTGAGCCGAGGAGGCTCCCAGAGTGTGTAT
GGTGTGGATCAGACTCACTCAGGGCTCCCCCTGCATCACATCAGGAGGATGTTCATGGGAGATTCATGC
CCTTATCTAGCAGGGAAGCCAAAGATGTTTTTTATTCAGAACTATGTGGTGTCAGAGGGCCAGCTGGAG
GACAGCAGCCTCTTGGAGGTGGATGGGCCAGCGATGAAGAATGTGGAATTCAAGGCTCAGAAGCGAGGG
CTGTGCACAGTTCACCGAGAAGCTGACTTCTTCTGGAGCCTGTGTACTGCGGACATGTCCCTGCTGGAG
CAGTCTCACAGCTCACCATCCCTGTACCTGCAGTGCCTCTCCCAGAAACTGAGACAAGAAAGAAAACGC
CCACTCCTGGATCTTCACATTGAACTCAATGGCTACATGTATGATTGGAACAGCAGAGTTTCTGCCAAG
GAGAAATATTATGTCTGGCTGCAGCACACTCTGAGAAAGAAACTTATCCTCTCCTACACATAA (SEQ
ID NO: 8)
CIRBP ENTREZ ID: 1153; OMIM: 602649; UNIPROT ID: CIRBP_HUMAN; ENSEMBL
ID: ENSG00000099622
ATGGCATCAGATGAAGGCAAACTTTTTGTTGGAGGGCTGAGTTTTGACACCAATGAGCAGTCGCTGGAG
CAGGTCTTCTCAAAGTACGGACAGATCTCTGAAGTGGTGGTTGTGAAAGACAGGGAGACCCAGAGATCT
CGGGGATTTGGGTTTGTCACCTTTGAGAACATTGACGACGCTAAGGATGCCATGATGGCCATGAATGGG
AAGTCTGTAGATGGACGGCAGATCCGAGTAGACCAGGCAGGCAAGTCGTCAGACAACCGATCCCGTGGG
TACCGTGGTGGCTCTGCCGGGGGCCGGGGCTTCTTCCGTGGGGGCCGAGGACGGGGCCGTGGGTTCTCT
AGAGGAGGAGGGGACCGAGGCTATGGGGGGAACCGGTTCGAGTCCAGGAGTGGGGGCTACGGAGGCTCC
AGAGACTACTATAGCAGCCGGAGTCAGAGTGGTGGCTACAGTGACCGGAGCTCGGGCGGGTCCTACAGA
GACAGTTATGACAGTTACGCTACACACAACGAGTAA (SEQ ID NO: 9)
CMTM7 Entrez ID: 112616; OMIM: 607890; Uniprot ID: CKLF7_HUMAN; ENSEMBL
ID: ENSG00000153551
ATGTCGCACGGAGCCGGGCTCGTCCGCACCACGTGCAGCAGCGGCAGCGCGCTCGGACCCGGGGCCGGC
GCGGCCCAGCCCAGCGCGAGCCCCTTGGAGGGGCTGCTGGACCTCAGCTACCCCCGCACCCACGCGGCC
CTGCTGAAAGTGGCGCAAATGGTCACCCTGCTGATTGCCTTCATCTGTGTGCGGAGCTCCCTGTGGACC
AACTACAGCGCCTACAGCTACTTTGAAGTGGTCACCATTTGCGACTTGATAATGATCCTCGCCTTTTAC
CTGGTCCACCTCTTCCGCTTCTACCGCGTGCTCACCTGTATCAGCTGGCCCCTGTCGGAACTTCTGCAC
TATTTAATCGGTACCCTGCTCCTCCTCATCGCCTCCATTGTGGCAGCTTCCAAGAGTTACAACCAGAGC
GGACTGGTAGCCGGAGCGATCTTTGGTTTCATGGCCACCTTCCTCTGCATGGCAAGCATATGGCTGTCC
TATAAGATCTCGTGTGTAACCCAGTCCACAGATGCAGCCGTCTGA (SEQ ID NO: 10)
CPNE3 ENTREZ ID: 8895; OMIM: 604207; UNIPROT ID: CPNE3_HUMAN; ENSEMBL
ID: ENSG00000085719
ATGGCTGCCCAGTGTGTCACAAAGGTGGCGCTGAATGTTTCCTGTGCCAATCTTTTGGATAAAGATATA
GGGTCAAAGTCAGACCCTTTATGTGTGTTGTTTTTGAATACAAGTGGTCAACAGTGGTATGAGGTTGAG
CGCACAGAAAGGATTAAGAATTGCTTGAATCCCCAATTTTCCAAGACATTTATTATTGATTACTACTTT
GAAGTGGTTCAGAAATTGAAATTTGGGGTTTATGACATCGACAACAAAACTATTGAGCTGAGTGATGAT
GACTTCTTAGGGGAATGTGAATGTACCCTTGGACAAATTGTTTCCAGCAAGAAGCTAACTCGACCACTG
GTGATGAAAACTGGCAGACCTGCAGGAAAAGGGAGCATTACGATTTCAGCTGAAGAAATAAAAGATAAT
AGAGTGGTCTTGTTTGAAATGGAAGCCAGAAAACTGGATAATAAGGATCTATTTGGAAAGTCAGACCCA
TACCTGGAATTCCACAAGCAGACATCTGATGGAAACTGGCTAATGGTTCATCGGACAGAGGTTGTTAAA
AACAACTTGAATCCTGTTTGGAGGCCTTTCAAGATCTCTCTTAACTCACTGTGTTACGGAGATATGGAC
AAAACCATTAAGGTGGAGTGTTATGATTATGACAATGATGGGTCACATGATCTCATTGGAACATTTCAG
ACCACCATGACAAAACTGAAAGAAGCCTCCAGAAGCTCACCTGTTGAATTTGAATGCATAAATGAGAAA
AAAAGGCAAAAGAAAAAAAGCTACAAGAATTCAGGTGTTATCAGTGTGAAACAGTGTGAGATTACAGTA
GAATGCACATTCCTTGACTATATAATGGGAGGATGTCAGCTGAATTTTACTGTGGGAGTGGACTTCACT
GGCTCCAATGGTGACCCAAGGTCTCCAGACTCCCTTCATTACATCAGCCCCAATGGCGTTAATGAGTAT
TTGACTGCTCTCTGGTCTGTGGGACTGGTCATTCAAGATTATGATGCTGATAAGATGTTTCCAGCTTTT
GGTTTTGGCGCTCAGATACCTCCTCAGTGGCAGGTATCACATGAATTTCCAATGAACTTCAACCCATCC
AATCCCTACTGCAATGGAATCCAAGGCATTGTAGAGGCGTATCGGTCTTGTCTTCCTCAGATAAAACTC
TATGGACCAACTAATTTTTCTCCAATCATAAATCACGTGGCCAGGTTTGCTGCTGCAGCCACGCAACAG
CAGACAGCTTCTCAATATTTTGTGCTTTTGATTATTACTGATGGTGTGATCACAGACCTTGATGAAACC
AGACAAGCTATAGTTAATGCCTCCAGGCTGCCTATGTCCATCATAATTGTTGGAGTTGGAGGTGCTGAC
TTCAGCGCCATGGAGTTTCTGGATGGTGATGGTGGAAGTCTCCGCTCCCCATTGGGCGAAGTGGCCATC
AGAGATATTGTCCAGTTTGTGCCTTTCAGACAGTTCCAGAATGCTCCAAAAGAAGCACTTGCTCAGTGT
GTCTTGGCAGAGATTCCCCAGCAGGTGGTGGGCTACTTCAATACATACAAACTCCTTCCTCCCAAGAAC
CCAGCCACGAAACAACAGAAGCAGTGA (SEQ ID NO: 11)
DKFZP564O0823 Entrez ID: 25849; OMIM:; Uniprot ID: PARM1_HUMAN;
ENSEMBL ID: ENSG00000169116
ATGGTCTACAAGACTCTCTTCGCTCTTTGCATCTTAACTGCAGGATGGAGGGTACAGAGTCTGCCTACA
TCAGCTCCTTTGTCTGTTTCTCTTCCGACAAACATTGTACCACCGACCACCATCTGGACTAGCTCTCCA
CAAAACACTGATGCAGACACTGCCTCCCCATCCAACGGCACTCACAACAACTCGGTGCTCCCAGTTACA
GCATCAGCCCCAACATCTCTGCTTCCTAAGAACATTTCCATAGAGTCCAGAGAAGAGGAGATCACCAGC
CCAGGTTCGAATTGGGAAGGCACAAACACAGACCCCTCACCTTCTGGGTTCTCGTCAACAAGCGGTGGA
GTCCACTTAACAACCACGTTGGAGGAACACAGCTCGGGCACTCCTGAAGCAGGCGTGGCAGCTACACTG
TCGCAGTCCGCTGCTGAGCCTCCCACACTCATCTCCCCTCAAGCTCCAGCCTCATCACCCTCATCCCTA
TCAACCTCACCACCTGAGGTCTTTTCTGCCTCCGTTACTACCAACCATAGCTCCACTGTGACCAGCACC
CAACCCACTGGAGCTCCAACTGCACCAGAGTCCCCGACAGAGGAGTCCAGCTCTGACCACACACCCACT
TCACATGCCACAGCTGAGCCAGTACCCCAGGAGAAAACACCCCCAACAACTGTGTCAGGCAAAGTGATG
TGTGAGCTCATAGACATGGAGACCACCACCACCTTTCCCAGGGTGATCATGCAGGAAGTAGAACATGCA
TTAAGTTCAGGCAGCATCGCCGCCATTACCGTGACAGTCATTGCCGTGGTGCTGCTGGTGTTTGGAGTT
GCAGCCTACCTAAAAATCAGGCATTCCTCCTATGGAAGACTTTTGGACGACCATGACTACGGGTCCTGG
GGAAACTACAACAACCCTCTGTACGATGACTCCTAA (SEQ ID NO: 12)
EMP3 Entrez ID: 2014; OMIM: 602335; Uniprot ID: EMP3_HUMAN; ENSEMBL ID:
ENSG00000142227
ATGTCACTCCTCTTGCTGGTGGTCTCAGCCCTTCACATCCTCATTCTTATACTGCTTTTCGTGGCCACT
TTGGACAAGTCCTGGTGGACTCTCCCTGGGAAAGAGTCCCTGAATCTCTGGTACGACTGCACGTGGAAC
AACGACACCAAAACATGGGCCTGCAGTAATGTCAGCGAGAATGGCTGGCTGAAGGCGGTGCAGGTCCTC
ATGGTGCTCTCCCTCATTCTCTGCTGTCTCTCCTTCATCCTGTTCATGTTCCAGCTCTACACCATGCGA
CGAGGAGGTCTCTTCTATGCCACCGGCCTCTGCCAGCTTTGCACCAGCGTGGCGGTGTTTACTGGCGCC
TTGATCTATGCCATTCACGCCGAGGAGATCCTGGAGAAGCACCCGCGAGGGGGCAGCTTCGGATACTGC
TTCGCCCTGGCCTGGGTGGCCTTCCCCCTCGCCCTGGTCAGCGGCATCATCTACATCCACCTACGGAAG
CGGGAGTGA (SEQ ID NO: 13)
FAM3C Entrez ID: 10447; OMIM: 608618; Uniprot ID: FAM3C_HUMAN; ENSEMBL
ID: ENSG00000196937
ATGAGGGTAGCAGGTGCTGCAAAGTTGGTGGTAGCTGTGGCAGTGTTTTTACTGACATTTTATGTTATT
TCTCAAGTATTTGAAATAAAAATGGATGCAAGTTTAGGAAATCTATTTGCAAGATCAGCATTGGACACA
GCTGCACGTTCTACAAAGCCTCCCAGATATAAGTGTGGGATCTCAAAAGCTTGCCCTGAGAAGCATTTT
GCTTTTAAAATGGCAAGTGGAGCAGCCAACGTGGTGGGACCCAAAATCTGCCTGGAAGATAATGTTTTA
ATGAGTGGTGTTAAGAATAATGTTGGAAGAGGGATCAATGTTGCCTTGGCAAATGGAAAAACAGGAGAA
GTATTAGACACTAAATATTTTGACATGTGGGGAGGAGATGTGGCACCATTTATTGAGTTTCTGAAGGCC
ATACAAGATGGAACAATAGTTTTAATGGGAACATACGATGATGGAGCAACCAAACTCAATGATGAGGCA
CGGCGGCTCATTGCTGATTTGGGGAGCACATCTATTACTAATCTTGGTTTTAGAGACAACTGGGTCTTC
TGTGGTGGGAAGGGCATTAAGACAAAAAGCCCTTTTGAACAGCACATAAAGAACAATAAGGATACAAAC
AAATATGAAGGATGGCCTGAAGTTGTAGAAATGGAAGGATGCATCCCCCAGAAGCAAGACTAA (SEQ
ID NO: 14)
FAM46A Entrez ID: 55603; OMIM: 611357; Uniprot ID: FA46A_HUMAN; ENSEMBL
ID: ENSG00000112773
ATGGCGGAGGGTGAAGGGTACTTCGCCATGTCTGAGGACGAGCTGGCCTGCAGCCCCTACATCCCCCTA
GGCGGCGACTTCGGCGGCGGCGACTTCGGCGGCGGCGACTTCGGCGGCGGCGACTTCGGCGGTGGCGGC
AGCTTCGGTGGGCATTGCTTGGACTATTGCGAAAGCCCTACGGCGCACTGCAATGTGCTGAACTGGGAG
CAAGTGCAGCGGCTGGACGGCATCCTGAGCGAGACCATTCCGATTCACGGGCGCGGCAACTTCCCCACG
CTCGAGCTGCAGCCGAGCCTGATCGTGAAGGTGGTGCGGCGGCGCCTGGCCGAGAAGCGCATTGGCGTC
CGCGACGTGCGCCTCAACGGCTCGGCAGCCAGCCATGTCCTGCACCAGGACAGCGGCCTGGGCTACAAG
GACCTGGACCTCATCTTCTGCGCCGACCTGCGCGGGGAAGGGGAGTTTCAGACTGTGAAGGACGTCGTG
CTGGACTGCCTGTTGGACTTCTTACCCGAGGGGGTGAACAAAGAGAAGATCACACCACTCACGCTCAAG
GAAGCTTATGTGCAGAAAATGGTTAAAGTGTGCAATGACTCTGACCGATGGAGTCTTATATCCCTGTCA
AACAACAGTGGCAAAAATGTGGAACTGAAATTTGTGGATTCCCTCCGGAGGCAGTTTGAATTCAGTGTA
GATTCTTTTCAAATCAAATTAGACTCTCTTCTGCTCTTTTATGAATGTTCAGAGAACCCAATGACTGAG
ACATTTCACCCCACAATAATCGGGGAGAGCGTCTATGGCGATTTCCAGGAAGCCTTTGATCACCTTTGT
AACAAGATCATTGCCACCAGGAACCCAGAGGAAATCCGAGGGGGAGGCCTGCTTAAGTACTGCAACCTC
TTGGTGAGGGGCTTTAGGCCCGCCTCTGATGAAATCAAGACCCTTCAAAGGTATATGTGTTCCAGGTTT
TTCATCGACTTCTCAGACATTGGAGAGCAGCAGAGAAAACTGGAGTCCTATTTGCAGAACCACTTTGTG
GGATTGGAAGACCGCAAGTATGAGTATCTCATGACCCTTCATGGAGTGGTAAATGAGAGCACAGTGTGC
CTGATGGGACATGAAAGAAGACAGACTTTAAACCTTATCACCATGCTGGCTATCCGGGTGTTAGCTGAC
CAAAATGTCATTCCTAATGTGGCTAATGTCACTTGCTATTACCAGCCAGCCCCCTATGTAGCAGATGCC
AACTTTAGCAATTACTACATTGCACAGGTTCAGCCAGTATTCACGTGCCAGCAACAGACCTACTCCACT
TGGCTACCCTGCAATTAA (SEQ ID NO: 15)
G1P3 Entrez ID: 2537; OMIM: 147572; Uniprot ID: IFI6_HUMAN; ENSEMBL ID:
ENSG00000126709
ATGCGGCAGAAGGCGGTATCGCTTTTCTTGTGCTACCTGCTGCTCTTCACTTGCAGTGGGGTGGAGGCA
GGTAAGAAAAAGTGCTCGGAGAGCTCGGACAGCGGCTCCGGGTTCTGGAAGGCCCTGACCTTCATGGCC
GTCGGAGGAGGACTCGCAGTCGCCGGGCTGCCCGCGCTGGGCTTCACCGGCGCCGGCATCGCGGCCAAC
TCGGTGGCTGCCTCGCTGATGAGCTGGTCTGCGATCCTGAATGGGGGCGGCGTGCCCGCCGGGGGGCTA
GTGGCCACGCTGCAGAGCCTCGGGGCTGGTGGCAGCAGCGTCGTCATAGGTAATATTGGTGCCCTGATG
GGCTACGCCACCCACAAGTATCTCGATAGTGAGGAGGATGAGGAGTAG (SEQ ID NO: 16)
GNA12 Entrez ID: 2768; OMIM: 604394; Uniprot ID: GNA12_HUMAN; ENSEMBL
ID: ENSG00000146535
ATGTCCGGGGTGGTGCGGACCCTCAGCCGCTGCCTGCTGCCGGCCGAGGCCGGCGGGGCCCGCGAGCGC
AGGGCGGGCAGCGGCGCGCGCGACGCGGAGCGCGAGGCCCGGAGGCGTAGCCGCGACATCGACGCGCTG
CTGGCCCGCGAGCGGCGCGCGGTCCGGCGCCTGGTGAAGATCCTGCTGCTGGGCGCGGGCGAGAGCGGC
AAGTCCACGTTCCTCAAGCAGATGCGCATCATCCACGGCCGCGAGTTCGACCAGAAGGCGCTGCTGGAG
TTCCGCGACACCATCTTCGACAACATCCTCAAGGGCTCAAGGGTTCTTGTTGATGCACGAGATAAGCTT
GGCATTCCTTGGCAGTATTCTGAAAATGAGAAGCATGGGATGTTCCTGATGGCCTTCGAGAACAAGGCG
GGGCTGCCTGTGGAGCCGGCCACCTTCCAGCTGTACGTCCCGGCCCTGAGCGCACTCTGGAGGGATTCT
GGCATCAGGGAGGCTTTCAGCCGGAGAAGCGAGTTTCAGCTGGGGGAGTCGGTGAAGTACTTCCTGGAC
AACTTGGACCGGATCGGCCAGCTGAATTACTTTCCTAGTAAGCAAGATATCCTGCTGGCTAGGAAAGCC
ACCAAGGGAATTGTGGAGCATGACTTCGTTATTAAGAAGATCCCCTTTAAGATGGTGGATGTGGGCGGC
CAGCGGTCCCAGCGCCAGAAGTGGTTCCAGTGCTTCGACGGGATCACGTCCATCCTGTTCATGGTCTCC
TCCAGCGAGTACGACCAGGTCCTCATGGAGGACAGGCGCACCAACCGGCTGGTGGAGTCCATGAACATC
TTCGAGACCATCGTCAACAACAAGCTCTTCTTCAACGTCTCCATCATTCTCTTCCTCAACAAGATGGAC
CTCCTGGTGGAGAAGGTGAAGACCGTGAGCATCAAGAAGCACTTCCCGGACTTCAGGGGCGACCCGCAC
AGGCTGGAGGACGTCCAGCGCTACCTGGTCCAGTGCTTCGACAGGAAGAGACGGAACCGCAGCAAGCCA
CTCTTCCACCACTTCACCACCGCCATCGACACCGAGAACGTCCGCTTCGTGTTCCATGCTGTGAAAGAC
ACCATCCTGCAGGAGAACCTGAAGGACATCATGCTGCAGTGA (SEQ ID NO: 17)
HSPB1 Entrez ID: 3315; OMIM: 602195; Uniprot ID: HSPB1_RUMAN; ENSEMBL
ID: ENSG00000106211
ATGACCGAGCGCCGCGTCCCCTTCTCGCTCCTGCGGGGCCCCAGCTGGGACCCCTTCCGCGACTGGTAC
CCGCATAGCCGCCTCTTCGACCAGGCCTTCGGGCTGCCCCGGCTGCCGGAGGAGTGGTCGCAGTGGTTA
GGCGGCAGCAGCTGGCCAGGCTACGTGCGCCCCCTGCCCCCCGCCGCCATCGAGAGCCCCGCAGTGGCC
GCGCCCGCCTACAGCCGCGCGCTCAGCCGGCAACTCAGCAGCGGGGTCTCGGAGATCCGGCACACTGCG
GACCGCTGGCGCGTGTCCCTGGATGTCAACCACTTCGCCCCGGACGAGCTGACGGTCAAGACCAAGGAT
GGCGTGGTGGAGATCACCGGCAAGCACGAGGAGCGGCAGGACGAGCATGGCTACATCTCCCGGTGCTTC
ACGCGGAAATACACGCTGCCCCCCGGTGTGGACCCCACCCAAGTTTCCTCCTCCCTGTCCCCTGAGGGC
ACACTGACCGTGGAGGCCCCCATGCCCAAGCTAGCCACGCAGTCCAACGAGATCACCATCCCAGTCACC
TTCGAGTCGCGGGCCCAGCTTGGGGGCCCAGAAGCTGCAAAATCCGATGAGACTGCCGCCAAGTAA
(SEQ ID NO: 18)
ID3 Entrez ID: 3399; OMIM: 600277; Uniprot ID: ID3_RUMAN; ENSEMBL ID:
ENSG00000117318
ATGAAGGCGCTGAGCCCGGTGCGCGGCTGCTACGAGGCGGTGTGCTGCCTGTCGGAACGCAGTCTGGCC
ATCGCCCGGGGCCGAGGGAAGGGCCCGGCAGCTGAGGAGCCGCTGAGCTTGCTGGACGACATGAACCAC
TGCTACTCCCGCCTGCGGGAACTGGTACCCGGAGTCCCGAGAGGCACTCAGCTTAGCCAGGTGGAAATC
CTACAGCGCGTCATCGACTACATTCTCGACCTGCAGGTAGTCCTGGCCGAGCCAGCCCCTGGACCCCCT
GATGGCCCCCACCTTCCCATCCAGACAGCCGAGCTCACTCCGGAACTTGTCATCTCCAACGACAAAAGG
AGCTTTTGCCACTGA (SEQ ID NO: 19)
IFITM2 Entrez ID: 10581; OMIM: 605578; Uniprot ID: IFM2_RUMAN; ENSEMBL
ID: ENSG00000185201
ATGAACCACATTGTGCAAACCTTCTCTCCTGTCAACAGCGGCCAGCCTCCCAACTACGAGATGCTCAAG
GAGGAGCAGGAAGTGGCTATGCTGGGGGTGCCCCACAACCCTGCTCCCCCGATGTCCACCGTGATCCAC
ATCCGCAGCGAGACCTCCGTGCCTGACCATGTGGTCTGGTCCCTGTTCAACACCCTCTTCATGAACACC
TGCTGCCTGGGCTTCATAGCATTCGCGTACTCCGTGAAGTCTAGGGACAGGAAGATGGTTGGCGACGTG
ACCGGGGCCCAGGCCTATGCCTCCACCGCCAAGTGCCTGAACATCTGGGCCCTGATTTTGGGCATCTTC
ATGACCATTCTGCTCATCATCATCCCAGTGTTGGTCGTCCAGGCCCAGCGATAG (SEQ ID NO: 20)
ITPR1 Entrez ID: 3708; OMIM: 147265; Uniprot ID: ITPR1_RUMAN; ENSEMBL
ID: ENSG00000150995
ATGTCTGACAAAATGTCTAGCTTCCTACATATTGGAGACATTTGTTCTCTGTACGCGGAGGGATCGACA
AATGGATTTATTAGCACCTTGGGCCTGGTTGATGATCGTTGTGTTGTACAGCCAGAAACCGGGGACCTT
AACAATCCACCTAAGAAATTCAGAGACTGCCTCTTTAAGCTATGTCCCATGAACCGCTACTCTGCCCAA
AAGCAGTTCTGGAAAGCCGCTAAGCCTGGGGCCAACAGCACCACAGACGCAGTGCTACTCAACAAACTG
CACCACGCTGCAGACTTGGAAAAGAAGCAGAATGAGACAGAAAACAGGAAATTGCTGGGGACCGTAATC
CAGTATGGCAATGTGATCCAGCTCCTGCATTTGAAAAGTAATAAATACCTAACAGTGAATAAGAGGCTT
CCTGCTCTGTTGGAGAAGAATGCCATGAGAGTCACATTGGACGAGGCTGGAAATGAAGGGTCCTGGTTT
TATATTCAGCCATTCTACAAGCTGCGATCCATTGGAGACAGCGTGGTCATAGGTGACAAGGTGGTTCTG
AACCCCGTCAATGCTGGTCAGCCCCTACATGCTAGCAGCCATCAACTGGTAGATAACCCAGGCTGCAAT
GAGGTCAATTCCGTCAACTGCAATACAAGCTGGAAAATAGTCCTTTTCATGAAATGGAGTGATAACAAA
GACGACATATTAAAGGGGGGTGACGTGGTGAGGCTGTTTCATGCTGAGCAGGAGAAGTTTCTCACCTGT
GACGAACACAGGAAGAAGCAGCACGTCTTCCTGAGAACCACGGGCCGGCAGTCGGCCACATCTGCCACC
AGTTCAAAAGCCCTGTGGGAGGTGGAGGTGGTCCAGCATGACCCATGTCGGGGCGGAGCAGGGTATTGG
AACAGCCTTTTCCGTTTCAAGCATCTGGCCACGGGGCATTACTTGGCAGCAGAGGTAGACCCTGACTTT
GAGGAAGAATGCCTGGAGTTTCAGCCCTCAGTGGACCCTGATCAGGACGCCTCTCGAAGTAGGTTGCGG
AATGCCCAAGAAAAGATGGTATACTCCCTGGTCTCTGTGCCTGAAGGCAATGACATCTCCTCCATTTTC
GAGCTAGATCCCACCACTCTGCGTGGAGGTGACAGCCTTGTCCCAAGGAACTCTTATGTTCGGCTCAGA
CACCTATGTACTAATACCTGGGTTCACAGCACAAATATTCCTATTGACAAGGAAGAAGAAAAGCCCGTG
ATGCTGAAAATTGGCACCTCTCCTGTGAAGGAGGATAAGGAAGCATTTGCCATAGTTCCGGTTTCTCCT
GCTGAAGTTCGGGACCTGGACTTTGCCAATGATGCCAGCAAGGTGCTGGGCTCCATTGCTGGGAAGCTA
GAGAAGGGCACCATCACCCAGAATGAAAGGAGGTCTGTAACCAAGCTGCTAGAAGATTTGGTTTACTTC
GTCACTGGTGGAACTAATTCTGGTCAAGATGTTCTCGAAGTTGTCTTCTCCAAGCCCAACAGAGAACGG
CAGAAACTGATGAGAGAACAGAATATTCTCAAGCAGATCTTCAAGTTGTTACAAGCCCCATTCACAGAC
TGCGGTGATGGCCCAATGCTTCGGCTGGAAGAGCTCGGGGACCAGCGGCACGCTCCTTTCAGACACATC
TGCCGGCTCTGCTACAGGGTGCTGAGACACTCGCAGCAAGACTACAGGAAGAACCAGGAGTATATAGCC
AAGCAGTTTGGCTTCATGCAGAAGCAGATTGGCTATGATGTGTTGGCTGAAGACACTATCACTGCCCTG
CTCCACAATAATCGGAAACTCCTGGAAAAACACATTACCGCGGCAGAGATTGACACATTTGTCAGCCTG
GTGCGAAAGAACAGGGAGCCCAGATTCTTAGATTACCTCTCCGACCTCTGTGTCTCCATGAACAAATCA
ATTCCAGTGACCCAGGAACTGATATGTAAAGCTGTGCTGAACCCCACCAACGCTGACATCCTGATTGAG
ACCAAGTTGGTTCTTTCTCGTTTTGAATTTGAAGGTGTCTCTTCCACTGGAGAGAATGCTCTGGAGGCA
GGAGAAGACGAGGAAGAGGTGTGGCTGTTTTGGAGGGACAGCAACAAAGAGATTCGCAGCAAGAGTGTG
AGGGAATTGGCTCAGGATGCTAAAGAAGGGCAGAAGGAGGACCGAGACGTTCTCAGCTACTACAGATAT
CAGCTGAACCTCTTTGCGAGGATGTGTCTGGACCGCCAATACCTGGCCATCAACGAAATCTCAGGCCAG
CTGGATGTCGATCTCATTCTCCGCTGCATGTCTGACGAGAACCTGCCCTATGACCTCAGGGCGTCCTTC
TGCCGCCTCATGCTTCACATGCATGTGGACCGAGATCCCCAGGAACAAGTCACCCCCGTGAAATATGCC
CGCCTCTGGTCGGAGATTCCCTCGGAGATCGCCATTGACGACTATGATAGTAGTGGAGCTTCCAAAGAT
GAAATTAAGGAGAGATTTGCTCAGACCATGGAGTTTGTGGAGGAGTATTTAAGAGATGTGGTTTGTCAG
AGGTTCCCTTTCTCTGATAAAGAGAAGAATAAGCTTACGTTTGAGGTTGTAAATTTAGCTAGGAATCTC
ATATACTTTGGTTTCTACAACTTCTCTGACCTTCTACGATTAACTAAGATCCTTCTGGCCATATTGGAC
TGTGTACATGTGACAACAATCTTCCCCATTAGCAAGATGGCGAAAGGAGAAGAGAATAAAGGCAGTAAC
GTGATGAGATCTATTCATGGCGTGGGAGAGCTGATGACCCAGGTGGTGCTCCGGGGAGGAGGCTTTTTG
CCCATGACTCCCATGGCTGCTGCCCCTGAAGGCAATGTGAAGCAGGCAGAGCCTGAGAAGGAGGACATC
ATGGTCATGGACACCAAGCTGAAGATCATTGAGATACTCCAGTTTATTTTGAATGTGAGGTTGGATTAT
AGGATCTCCTGCCTCCTGTGTATATTTAAGCGAGAGTTTGATGAAAGCAATTCCCAGACTTCAGAAACA
TCCTCCGGAAACAGCAGCCAAGAAGGGCCAAGTAATGTACCAGGTGCTCTTGACTTTGAACACATTGAA
GAACAAGCAGAAGGCATCTTTGGAGGAAGTGAGGAGAACACCCCACTGGACTTGGATGACCACGGCGGC
AGAACCTTTCTCCGTGTCCTGCTCCACTTGACGATGCATGACTACCCACCCCTGGTGTCAGGGGCCCTG
CAGCTCCTCTTCCGGCACTTCAGCCAGAGGCAGGAGGTGCTCCAGGCCTTCAAACAGGTTCAACTGCTG
GTTACCAGCCAAGATGTGGACAACTACAAACAGATCAAACAAGACTTGGATCAACTGAGGTCCATCGTG
GAAAAGTCAGAGCTTTGGGTGTACAAAGGGCAGGGCCCCGATGAGACTATGGATGGTGCATCTGGAGAA
AATGAACATAAGAAAACGGAGGAGGGAAATAACAAGCCACAAAAGCATGAAAGCACCAGCAGCTACAAC
TACAGAGTGGTCAAAGAGATTTTGATTCGGCTTAGCAAACTCTGTGTTCAAGAGAGTGCCTCAGTGAGA
AAGAGCAGGAAGCAGCAACAGCGTCTGCTCCGGAACATGGGCGCGCACGCCGTGGTGCTGGAGCTGCTG
CAGATTCCCTATGAGAAGGCCGAAGATACCAAGATGCAAGAGATAATGAGGTTGGCTCATGAATTTTTG
CAGAATTTCTGCGCAGGCAACCAGCAGAATCAAGCTTTGCTACATAAACACATAAACCTGTTTCTCAAC
CCAGGGATCCTGGAGGCAGTAACCATGCAGCACATCTTCATGAACAATTTCCAGCTTTGCAGTGAGATC
AACGAGAGAGTTGTTCAGCACTTCGTTCACTGCATAGAGACTCACGGTCGGAATGTCCAGTATATAAAG
TTCTTACAGACAATTGTCAAGGCAGAAGGGAAATTTATTAAAAAATGCCAAGACATGGTTATGGCCGAG
CTGGTCAATTCGGGAGAGGATGTCCTCGTGTTCTACAACGACAGAGCCTCTTTCCAGACTCTGATCCAG
ATGATGCGGTCAGAACGGGATCGGATGGATGAGAACAGCCCTCTCATGTACCACATCCACTTGGTCGAG
CTCCTGGCTGTGTGCACGGAGGGTAAGAATGTCTACACAGAGATCAAGTGCAACTCCCTGCTCCCGCTG
GATGACATCGTTCGCGTGGTGACCCACGAGGACTGCATCCCTGAGGTTAAAATTGCATACATTAACTTC
CTGAATCACTGCTATGTGGATACAGAGGTGGAAATGAAGGAGATTTATACCAGCAATCACATGTGGAAA
TTGTTTGAGAATTTCCTTGTAGACATCTGCAGGGCCTGTAACAACACTAGTGACAGGAAACATGCAGAC
TCGATTTTGGAGAAGTATGTCACCGAAATCGTCATGAGTATTGTTACTACTTTCTTCAGCTCTCCCTTC
TCAGACCAGAGTACGACTTTGCAGACTCGCCAGCCTGTCTTTGTGCAACTGCTGCAAGGCGTGTTCAGG
GTTTACCACTGCAACTGGTTAATGCCAAGCCAAAAAGCCTCCGTGGAGAGCTGTATTCGGGTGCTGTCT
GATGTAGCCAAGAGCCGGGCCATTGCCATTCCCGTGGACCTGGACAGCCAAGTCAACAACCTCTTTCTC
AAGTCCCACAGCATTGTGCAGAAAACAGCCATGAACTGGCGGCTCTCAGCCCGCAATGCCGCACGCAGG
GACTCTGTTCTGGCAGCTTCCAGAGACTACCGGAATATCATTGAGAGATTGCAGGACATCGTCTCCGCG
CTGGAGGACCGTCTCAGGCCCCTGGTGCAGGCAGAGTTATCTGTGCTCGTGGATGTTCTCCACAGACCC
GAGCTGCTTTTCCCAGAGAACACAGACGCCAGAAGGAAATGTGAAAGTGGCGGTTTCATTTGCAAGTTA
ATAAAGCATACAAAACAGCTGCTAGAAGAAAATGAAGAGAAGCTCTGCATTAAGGTCCTACAGACCCTG
AGGGAAATGATGACCAAAGATAGAGGCTATGGAGAAAAGGGTGAGGCGCTCAGGCAAGTTCTGGTCAAC
CGTTACTATGGAAACGTCAGACCTTCGGGACGAAGAGAGAGCCTTACCAGCTTTGGCAATGGCCCACTG
TCAGCAGGAGGACCCGGCAAGCCCGGGGGAGGAGGGGGAGGTTCCGGATCCAGCTCTATGAGCAGGGGT
GAGATGAGTCTGGCCGAGGTTCAGTGTCACCTTGACAAGGAGGGGGCTTCCAATCTAGTTATCGACCTC
ATCATGAACGCATCCAGTGACCGAGTGTTCCATGAAAGCATTCTCCTGGCCATTGCCCTTCTGGAAGGA
GGCAACACCACCATCCAGCACTCCTTTTTCTGTCGCTTGACAGAAGATAAGAAGTCAGAGAAATTCTTT
AAGGTGTTTTATGACCGGATGAAGGTGGCCCAGCAAGAAATCAAAGCAACAGTGACAGTGAACACCAGT
GACTTGGGAAATAAAAAGAAAGACGATGAGGTAGACAGGGATGCCCCATCACGGAAAAAAGCTAAAGAG
CCCACAACACAGATAACAGAAGAGGTCCGGGATCAGCTCCTGGAGGCCTCCGCTGCCACCAGGAAAGCC
TTCACCACTTTCAGGAGGGAGGCTGATCCCGACGACCACTACCAGCCTGGAGAGGGCACCCAGGCCACT
GCCGACAAGGCCAAGGACGACCTGGAGATGAGCGCGGTCATCACCATCATGCAGCCCATCCTCCGCTTC
CTTCAGCTCCTGTGTGAAAACCACAACCGAGACCTGCAGAACTTCCTCCGTTGCCAAAATAACAAGACC
AACTACAATTTGGTATGTGAGACCCTGCAGTTTCTGGACTGTATTTGTGGAAGCACAACTGGAGGCCTT
GGTCTTCTGGGCTTGTATATAAATGAAAAGAACGTAGCGCTTATCAACCAAACCCTGGAAAGTCTGACC
GAATACTGTCAAGGACCTTGCCATGAGAACCAGAACTGCATAGCCACCCATGAATCCAATGGCATTGAC
ATCATCACAGCCCTGATCCTCAATGATATCAATCCTTTGGGAAAGAAGAGGATGGACCTTGTGTTAGAA
CTGAAGAACAATGCCTCGAAGTTGCTCCTGGCCATCATGGAAAGCAGGCACGACAGTGAAAACGCAGAG
AGGATACTTTATAACATGAGGCCCAAGGAACTGGTGGAAGTGATCAAGAAAGCCTACATGCAAGGTGAA
GTGGAATTTGAGGATGGAGAAAACGGTGAGGATGGGGCGGCGTCCCCCAGGAACGTGGGGCACAACATC
TACATATTAGCCCATCAGTTGGCTCGGCATAACAAAGAACTTCAGAGCATGCTGAAACCTGGTGGCCAA
GTGGACGGAGATGAAGCCCTGGAGTTTTATGCCAAGCACACGGCGCAGATAGAGATTGTCAGATTAGAC
CGAACAATGGAACAGATAGTCTTTCCCGTGCCCAGCATATGTGAATTCCTAACCAAGGAGTCAAAACTA
CGAATTTACTATACTACAGAGAGAGACGAACAAGGCAGCAAAATCAATGATTTCTTTCTGCGGTCTGAA
GACCTCTTCAATGAAATGAATTGGCAGAAGAAACTGAGAGCCCAGCCCGTGTTGTACTGGTGTGCCCGC
AACATGTCTTTCTGGAGCAGCATTTCGTTTAACCTGGCCGTCCTGATGAACCTGCTGGTGGCGTTTTTC
TACCCGTTTAAGGGAGTCCGAGGAGGAACCCTGGAGCCCCACTGGTCGGGACTCCTGTGGACAGCCATG
CTCATCTCTCTGGCCATCGTCATTGCCCTCCCCAAGCCCCATGGCATCCGGGCCTTAATTGCCTCCACA
ATTCTACGACTGATATTTTCAGTCGGGTTACAACCCACGTTGTTTCTTCTGGGCGCTTTCAATGTATGC
AATAAAATCATCTTTCTAATGAGCTTTGTGGGCAACTGTGGGACATTCACAAGAGGCTACCGAGCCATG
GTTCTGGATGTTGAGTTCCTCTATCATTTGTTGTATCTGGTGATCTGTGCCATGGGGCTCTTTGTCCAT
GAATTCTTCTACAGTCTGCTGCTTTTTGATTTAGTGTACAGAGAAGAGACTTTGCTTAATGTCATTAAA
AGTGTCACTCGCAATGGACGGTCCATCATCCTGACAGCAGTTCTGGCTCTGATCCTCGTTTACCTGTTC
TCAATAGTGGGCTATCTTTTCTTCAAGGATGACTTTATCTTGGAAGTAGATAGGCTGCCCAATGAAACA
GCTGTTCCAGAAACCGGCGAGAGTTTGGCAAGCGAGTTCCTGTTCTCCGATGTGTGTAGGGTGGAGAGT
GGGGAGAACTGCTCCTCTCCTGCACCCAGAGAAGAGCTGGTCCCTGCAGAAGAGACGGAACAGGATAAA
GAGCACACATGTGAGACGCTGCTGATGTGCATTGTCACTGTGCTGAGTCACGGGCTGCGGAGCGGGGGT
GGAGTAGGAGATGTACTCAGGAAGCCGTCCAAAGAGGAACCCCTGTTTGCTGCTAGAGTTATTTATGAC
CTCTTGTTCTTCTTCATGGTCATCATCATTGTTCTTAACCTGATTTTTGGGGTTATCATTGACACTTTT
GCTGACCTGAGGAGTGAGAAGCAGAAGAAGGAAGAGATCTTGAAGACCACGTGCTTTATCTGTGGCTTG
GAAAGAGACAAGTTTGACAACAAGACTGTCACCTTTGAAGAGCACATCAAGGAAGAACACAACATGTGG
CACTATCTGTGCTTCATCGTCCTGGTGAAAGTAAAGGACTCCACCGAATATACTGGGCCTGAGAGTTAC
GTGGCAGAAATGATCAAGGAAAGAAACCTTGACTGGTTCCCCAGGATGAGAGCCATGTCATTGGTCAGC
AGTGATTCTGAAGGAGAACAGAATGAGCTGAGAAACCTGCAGGAGAAGCTGGAGTCCACCATGAAACTT
GTCACGAACCTTTCTGGCCAGCTGTCGGAATTAAAGGATCAGATGACAGAACAAAGGAAGCAGAAACAA
AGAATTGGTCTTCTAGGACATCCTCCTCACATGAATGTCAACCCACAACAACCAGCATAA (SEQ
ID NO: 21)
JUN Entrez ID: 3725; OMIM: 165160; Uniprot ID: JUN_HUMAN; ENSEMBL ID:
ENSG00000177606
ATGACTGCAAAGATGGAAACGACCTTCTATGACGATGCCCTCAACGCCTCGTTCCTCCCGTCCGAGAGC
GGACCTTATGGCTACAGTAACCCCAAGATCCTGAAACAGAGCATGACCCTGAACCTGGCCGACCCAGTG
GGGAGCCTGAAGCCGCACCTCCGCGCCAAGAACTCGGACCTCCTCACCTCGCCCGACGTGGGGCTGCTC
AAGCTGGCGTCGCCCGAGCTGGAGCGCCTGATAATCCAGTCCAGCAACGGGCACATCACCACCACGCCG
ACCCCCACCCAGTTCCTGTGCCCCAAGAACGTGACAGATGAGCAGGAGGGCTTCGCCGAGGGCTTCGTG
CGCGCCCTGGCCGAACTGCACAGCCAGAACACGCTGCCCAGCGTCACGTCGGCGGCGCAGCCGGTCAAC
GGGGCAGGCATGGTGGCTCCCGCGGTAGCCTCGGTGGCAGGGGGCAGCGGCAGCGGCGGCTTCAGCGCC
AGCCTGCACAGCGAGCCGCCGGTCTACGCAAACCTCAGCAACTTCAACCCAGGCGCGCTGAGCAGCGGC
GGCGGGGCGCCCTCCTACGGCGCGGCCGGCCTGGCCTTTCCCGCGCAACCCCAGCAGCAGCAGCAGCCG
CCGCACCACCTGCCCCAGCAGATGCCCGTGCAGCACCCGCGGCTGCAGGCCCTGAAGGAGGAGCCTCAG
ACAGTGCCCGAGATGCCCGGCGAGACACCGCCCCTGTCCCCCATCGACATGGAGTCCCAGGAGCGGATC
AAGGCGGAGAGGAAGCGCATGAGGAACCGCATCGCTGCCTCCAAGTGCCGAAAAAGGAAGCTGGAGAGA
ATCGCCCGGCTGGAGGAAAAAGTGAAAACCTTGAAAGCTCAGAACTCGGAGCTGGCGTCCACGGCCAAC
ATGCTCAGGGAACAGGTGGCACAGCTTAAACAGAAAGTCATGAACCACGTTAACAGTGGGTGCCAACTC
ATGCTAACGCAGCAGTTGCAAACATTTTGA (SEQ ID NO: 22)
LY96 Entrez ID: 23643; OMIM: 605243; Uniprot ID: LY96_HUMAN; ENSEMBL
ID: ENSG00000154589
ATGTTACCATTTCTGTTTTTTTCCACCCTGTTTTCTTCCATATTTACTGAAGCTCAGAAGCAGTATTGG
GTCTGCAACTCATCCGATGCAAGTATTTCATACACCTACTGTGGGAGAGATTTAAAGCAATTATATTTC
AATCTCTATATAACTGTCAACACCATGAATCTTCCAAAGCGCAAAGAAGTTATTTGCCGAGGATCTGAT
GACGATTACTCTTTTTGCAGAGCTCTGAAGGGAGAGACTGTGAATACAACAATATCATTCTCCTTCAAG
GGAATAAAATTTTCTAAGGGAAAATACAAATGTGTTGTTGAAGCTATTTCTGGGAGCCCAGAAGAAATG
CTCTTTTGCTTGGAGTTTGTCATCCTACACCAACCTAATTCAAATTAG (SEQ ID NO: 23)
MAP2K1 Entrez ID: 5604; OMIM: 176872; Uniprot ID: MP2K1_HUMAN; ENSEMBL
ID: ENSG00000169032
ATGCCCAAGAAGAAGCCGACGCCCATCCAGCTGAACCCGGCCCCCGACGGCTCTGCAGTTAACGGGACC
AGCTCTGCGGAGACCAACTTGGAGGCCTTGCAGAAGAAGCTGGAGGAGCTAGAGCTTGATGAGCAGCAG
CGAAAGCGCCTTGAGGCCTTTCTTACCCAGAAGCAGAAGGTGGGAGAACTGAAGGATGACGACTTTGAG
AAGATCAGTGAGCTGGGGGCTGGCAATGGCGGTGTGGTGTTCAAGGTCTCCCACAAGCCTTCTGGCCTG
GTCATGGCCAGAAAGCTAATTCATCTGGAGATCAAACCCGCAATCCGGAACCAGATCATAAGGGAGCTG
CAGGTTCTGCATGAGTGCAACTCTCCGTACATCGTGGGCTTCTATGGTGCGTTCTACAGCGATGGCGAG
ATCAGTATCTGCATGGAGCACATGGATGGAGGTTCTCTGGATCAAGTCCTGAAGAAAGCTGGAAGAATT
CCTGAACAAATTTTAGGAAAAGTTAGCATTGCTGTAATAAAAGGCCTGACATATCTGAGGGAGAAGCAC
AAGATCATGCACAGAGATGTCAAGCCCTCCAACATCCTAGTCAACTCCCGTGGGGAGATCAAGCTCTGT
GACTTTGGGGTCAGCGGGCAGCTCATCGACTCCATGGCCAACTCCTTCGTGGGCACAAGGTCCTACATG
TCGCCAGAAAGACTCCAGGGGACTCATTACTCTGTGCAGTCAGACATCTGGAGCATGGGACTGTCTCTG
GTAGAGATGGCGGTTGGGAGGTATCCCATCCCTCCTCCAGATGCCAAGGAGCTGGAGCTGATGTTTGGG
TGCCAGGTGGAAGGAGATGCGGCTGAGACCCCACCCAGGCCAAGGACCCCCGGGAGGCCCCTTAGCTCA
TACGGAATGGACAGCCGACCTCCCATGGCAATTTTTGAGTTGTTGGATTACATAGTCAACGAGCCTCCT
CCAAAACTGCCCAGTGGAGTGTTCAGTCTGGAATTTCAAGATTTTGTGAATAAATGCTTAATAAAAAAC
CCCGCAGAGAGAGCAGATTTGAAGCAACTCATGGTTCATGCTTTTATCAAGAGATCTGATGCTGAGGAA
GTGGATTTTGCAGGTTGGCTCTGCTCCACCATCGGCCTTAACCAGCCCAGCACACCAACCCATGCTGCT
GGCGTCTAA (SEQ ID NO: 24)
MCL1 Entrez ID: 7249; OMIM: 191092; Uniprot ID: TSC2_HUMAN; ENSEMBL ID:
ENSG00000103197
ATGTTTGGCCTCAAAAGAAACGCGGTAATCGGACTCAACCTCTACTGTGGGGGGGCCGGCTTGGGGGCC
GGCAGCGGCGGCGCCACCCGCCCGGGAGGGCGACTTTTGGCCACCGGCGCCAAGGACACAAAGCCAATG
GGCAGGTCTGGGGCCACCAGCAGGAAGGCGCTGGAGACCTTACGACGGGTTGGGGATGGCGTGCAGCGC
AACCACGAGACGGCCTTCCAAGGCATGCTTCGGAAACTGGACATCAAAAACGAAGACGATGTGAAATCG
TTGTCTCGAGTGATGATCCATGTTTTCAGCGACGGCGTAACAAACTGGGGCAGGATTGTGACTCTCATT
TCTTTTGGTGCCTTTGTGGCTAAACACTTGAAGACCATAAACCAAGAAAGCTGCATCGAACCATTAGCA
GAAAGTATCACAGACGTTCTCGTAAGGACAAAACGGGACTGGCTAGTTAAACAAAGAGGCTGGGATGGG
TTTGTGGAGTTCTTCCATGTAGAGGACCTAGAAGGTGGCATCAGGAATGTGCTGCTGGCTTTTGCAGGT
GTTGCTGGAGTAGGAGCTGGTTTGGCATATCTAATAAGATAG (SEQ ID NO: 25)
MT2A Entrez ID: 4502; OMIM: 156360; Uniprot ID: MT2_HUMAN; ENSEMBL ID:
ENSG00000125148
ATGGATCCCAACTGCTCCTGCGCCGCCGGTGACTCCTGCACCTGCGCCGGCTCCTGCAAATGCAAAGAG
TGCAAATGCACCTCCTGCAAGAAAAGCTGCTGCTCCTGCTGCCCTGTGGGCTGTGCCAAGTGTGCCCAG
GGCTGCATCTGCAAAGGGGCGTCGGACAAGTGCAGCTGCTGCGCCTGA (SEQ ID NO: 26)
NEFM Entrez ID: 4741; OMIM: 162250; Uniprot ID: NFM_HUMAN; ENSEMBL ID:
ENSG00000104722
ATGGCTCGTCATTTGCGCGAATACCAGGACCTCCTCAACGTCAAGATGGCTCTGGATATAGAAATCGCT
GCGTACAGAAAACTCCTGGAGGGTGAAGAGACTAGATTTAGCACATTTGCAGGAAGCATCACTGGGCCA
CTGTATACACACCGACCCCCAATCACAATATCCAGTAAGATTCAGAAACCCAAGGTGGAAGCTCCCAAG
CTTAAGGTCCAACACAAATTTGTCGAGGAGATCATAGAGGAAACCAAAGTGGAGGATGAGAAGTCAGAA
ATGGAAGAGGCCCTGACAGCCATTACAGAGGAATTGGCCGTTTCCATGAAGGAAGAGAAGAAAGAAGCA
GCAGAAGAAAAGGAAGAGGAACCCGAAGCTGAAGAAGAAGAAGTAGCTGCCAAAAAGTCTCCAGTGAAA
GCAACTGCACCTGAAGTTAAAGAAGAGGAAGGGGAAAAGGAGGAAGAAGAAGGCCAGGAAGAAGAGGAG
GAAGAAGATGAGGGAGCTAAGTCAGACCAAGCCGAAGAGGGAGGATCCGAGAAGGAAGGCTCTAGTGAA
AAAGAGGAAGGTGAGCAGGAAGAAGGAGAAACAGAAGCTGAAGCTGAAGGAGAGGAAGCCGAAGCTAAA
GAGGAAAAGAAAGTGGAGGAAAAGAGTGAGGAAGTGGCTACCAAGGAGGAGCTGGTGGCAGATGCCAAG
GTGGAAAAGCCAGAAAAAGCCAAGTCTCCTGTGCCAAAATCACCAGTGGAAGAGAAAGGCAAGTCTCCT
GTGCCCAAGTCACCAGTGGAAGAGAAAGGCAAGTCTCCTGTGCCCAAGTCACCAGTGGAAGAGAAAGGC
AAGTCTCCTGTGCCGAAATCACCAGTGGAAGAGAAAGGCAAGTCTCCTGTGTCAAAATCACCAGTGGAA
GAGAAAGCCAAATCTCCTGTGCCAAAATCACCAGTGGAAGAGGCAAAGTCAAAAGCAGAAGTGGGGAAA
GGTGAACAGAAAGAGGAAGAAGAAAAGGAAGTCAAGGAAGCTCCCAAGGAAGAGAAGGTAGAGAAAAAG
GAAGAGAAACCAAAGGATGTGCCAGAGAAGAAGAAAGCTGAGTCCCCTGTAAAGGAGGAAGCTGTGGCA
GAGGTGGTCACCATCACCAAATCGGTAAAGGTGCACTTGGAGAAAGAGACCAAAGAAGAGGGGAAGCCA
CTGCAGCAGGAGAAAGAGAAGGAGAAAGCGGGAGGAGAGGGAGGAAGTGAGGAGGAAGGGAGTGATAAA
GGTGCCAAGGGATCCAGGAAGGAAGACATAGCTGTCAATGGGGAGGTAGAAGGAAAAGAGGAGGTAGAG
CAGGAGACCAAGGAAAAAGGCAGTGGGAGGGAAGAGGAGAAAGGCGTTGTCACCAATGGCCTAGACTTG
AGCCCAGCAGATGAAAAGAAGGGGGGTGATAAAAGTGAGGAGAAAGTGGTGGTGACCAAAACGGTAGAA
AAAATCACCAGTGAGGGGGGAGATGGTGCTACCAAATACATCACTAAATCTGTAACCGTCACTCAAAAG
GTTGAAGAGCATGAAGAGACCTTTGAGGAGAAACTAGTGTCTACTAAAAAGGTAGAAAAAGTCACTTCA
CACGCCATAGTAAAGGAAGTCACCCAGAGTGACTAA (SEQ ID NO: 27)
NQO1 Entrez ID: 1728; OMIM: 125860; Uniprot ID: NQO1_HUMAN; ENSEMBL ID:
ENSG00000181019
ATGGTCGGCAGAAGAGCACTGATCGTACTGGCTCACTCAGAGAGGACGTCCTTCAACTATGCCATGAAG
GAGGCTGCTGCAGCGGCTTTGAAGAAGAAAGGATGGGAGGTGGTGGAGTCGGACCTCTATGCCATGAAC
TTCAATCCCATCATTTCCAGAAAGGACATCACAGGTAAACTGAAGGACCCTGCGAACTTTCAGTATCCT
GCCGAGTCTGTTCTGGCTTATAAAGAAGGCCATCTGAGCCCAGATATTGTGGCTGAACAAAAGAAGCTG
GAAGCCGCAGACCTTGTGATATTCCAGTTCCCCCTGCAGTGGTTTGGAGTCCCTGCCATTCTGAAAGGC
TGGTTTGAGCGAGTGTTCATAGGAGAGTTTGCTTACACTTACGCTGCCATGTATGACAAAGGACCCTTC
CGGAGTAAGAAGGCAGTGCTTTCCATCACCACTGGTGGCAGTGGCTCCATGTACTCTCTGCAAGGGATC
CACGGGGACATGAATGTCATTCTCTGGCCAATTCAGAGTGGCATTCTGCATTTCTGTGGCTTCCAAGTC
TTAGAACCTCAACTGACATATAGCATTGGGCACACTCCAGCAGACGCCCGAATTCAAATCCTGGAAGGA
TGGAAGAAACGCCTGGAGAATATTTGGGATGAGACACCACTGTATTTTGCTCCAAGCAGCCTCTTTGAC
CTAAACTTCCAGGCAGGATTCTTAATGAAAAAAGAGGTACAGGATGAGGAGAAAAACAAGAAATTTGGC
CTTTCTGTGGGCCATCACTTGGGCAAGTCCATCCCAACTGACAACCAGATCAAAGCTAGAAAATGA
(SEQ ID NO: 28)
P4HA1 Entrez ID: 5033; OMIM: 176710; Uniprot ID: P4HA1_HUMAN; ENSEMBL
ID: ENSG00000122884
ATGATCTGGTATATATTAATTATAGGAATTCTGCTTCCCCAGTCTTTGGCTCATCCAGGCTTTTTTACT
TCAATTGGTCAGATGACTGATTTGATCCATACTGAGAAAGATCTGGTGACTTCTCTGAAAGATTATATT
AAGGCAGAAGAGGACAAGTTAGAACAAATAAAAAAATGGGCAGAGAAGTTAGATCGGCTAACTAGTACA
GCGACAAAAGATCCAGAAGGATTTGTTGGGCATCCAGTAAATGCATTCAAATTAATGAAACGTCTGAAT
ACTGAGTGGAGTGAGTTGGAGAATCTGGTCCTTAAGGATATGTCAGATGGCTTTATCTCTAACCTAACC
ATTCAGAGACAGTACTTTCCTAATGATGAAGATCAGGTTGGGGCAGCCAAAGCTCTGTTACGTCTCCAG
GATACCTACAATTTGGATACAGATACCATCTCAAAGGGTAATCTTCCAGGAGTGAAACACAAATCTTTT
CTAACGGCTGAGGACTGCTTTGAGTTGGGCAAAGTGGCCTATACAGAAGCAGATTATTACCATACGGAA
CTGTGGATGGAACAAGCCCTAAGGCAACTGGATGAAGGCGAGATTTCTACCATAGATAAAGTCTCTGTT
CTAGATTATTTGAGCTATGCGGTATATCAGCAGGGAGACCTGGATAAGGCACTTTTGCTCACAAAGAAG
CTTCTTGAACTAGATCCTGAACATCAGAGAGCTAATGGTAACTTAAAATATTTTGAGTATATAATGGCT
AAAGAAAAAGATGTCAATAAGTCTGCTTCAGATGACCAATCTGATCAGAAAACTACACCAAAGAAAAAA
GGGGTTGCTGTGGATTACCTGCCAGAGAGACAGAAGTACGAAATGCTGTGCCGTGGGGAGGGTATCAAA
ATGACCCCTCGGAGACAGAAAAAACTCTTTTGCCGCTACCATGATGGAAACCGTAATCCTAAATTTATT
CTGGCTCCAGCTAAACAGGAGGATGAATGGGACAAGCCTCGTATTATTCGCTTCCATGATATTATTTCT
GATGCAGAAATTGAAATCGTCAAAGACCTAGCAAAACCAAGGCTGAGCCGAGCTACAGTACATGACCCT
GAGACTGGAAAATTGACCACAGCACAGTACAGAGTATCTAAGAGTGCCTGGCTCTCTGGCTATGAAAAT
CCTGTGGTGTCTCGAATTAATATGAGAATACAAGATCTAACAGGACTAGATGTTTCCACAGCAGAGGAA
TTACAGGTAGCAAATTATGGAGTTGGAGGACAGTATGAACCCCATTTTGACTTTGCACGGAAAGATGAG
CCAGATGCTTTCAAAGAGCTGGGGACAGGAAATAGAATTGCTACATGGCTGTTTTATATGAGTGATGTG
TCTGCAGGAGGAGCCACTGTTTTTCCTGAAGTTGGAGCTAGTGTTTGGCCCAAAAAAGGAACTGCTGTT
TTCTGGTATAATCTGTTTGCCAGTGGAGAAGGAGATTATAGTACACGGCATGCAGCCTGTCCAGTGCTA
GTTGGCAACAAATGGGTATCCAATAAATGGCTCCATGAACGTGGACAAGAATTTCGAAGACCTTGTACG
TTGTCAGAATTGGAATGA (SEQ ID NO: 29)
PLOD2 Entrez ID: 5352; OMIM: 601865; Uniprot ID: PLOD2_HUMAN; ENSEMBL
ID: ENSG00000152952
ATGGGGGGATGCACGGTGAAGCCTCAGCTGCTGCTCCTGGCGCTCGTCCTCCACCCCTGGAATCCCTGT
CTGGGTGCGGACTCGGAGAAGCCCTCGAGCATCCCCACAGATAAATTATTAGTCATAACTGTAGCAACA
AAAGAAAGTGATGGATTCCATCGATTTATGCAGTCAGCCAAATATTTCAATTATACTGTGAAGGTCCTT
GGTCAAGGAGAAGAATGGAGAGGTGGTGATGGAATTAATAGTATTGGAGGGGGCCAGAAAGTGAGATTA
ATGAAAGAAGTCATGGAACACTATGCTGATCAAGATGATCTGGTTGTCATGTTTACTGAATGCTTTGAT
GTCATATTTGCTGGTGGTCCAGAAGAAGTTCTAAAAAAATTCCAAAAGGCAAACCACAAAGTGGTCTTT
GCAGCAGATGGAATTTTGTGGCCAGATAAAAGACTAGCAGACAAGTATCCTGTTGTGCACATTGGGAAA
CGCTATCTGAATTCAGGAGGATTTATTGGCTATGCTCCATATGTCAACCGTATAGTTCAACAATGGAAT
CTCCAGGATAATGATGATGATCAGCTCTTTTACACTAAAGTTTACATTGATCCACTGAAAAGGGAAGCT
ATTAACATCACATTGGATCACAAATGCAAAATTTTCCAGACCTTAAATGGAGCTGTAGATGAAGTTGTT
TTAAAATTTGAAAATGGCAAAGCCAGAGCTAAGAATACATTTTATGAAACATTACCAGTGGCAATTAAT
GGAAATGGACCCACCAAGATTCTCCTGAATTATTTTGGAAACTATGTACCCAATTCATGGACACAGGAT
AATGGCTGCACTCTTTGTGAATTCGATACAGTCGACTTGTCTGCAGTAGATGTCCATCCAAACGTATCA
ATAGGTGTTTTTATTGAGCAACCAACCCCTTTTCTACCTCGGTTTCTGGACATATTGTTGACACTGGAT
TACCCAAAAGAAGCACTTAAACTTTTTATTCATAACAAAGAAGTTTATCATGAAAAGGACATCAAGGTA
TTTTTTGATAAAGCTAAGCATGAAATCAAAACTATAAAAATAGTAGGACCAGAAGAAAATCTAAGTCAA
GCGGAAGCCAGAAACATGGGAATGGACTTTTGCCGTCAGGATGAAAAGTGTGATTATTACTTTAGTGTG
GATGCAGATGTTGTTTTGACAAATCCAAGGACTTTAAAAATTTTGATTGAACAAAACAGAAAGATCATT
GCTCCTCTTGTAACTCGTCATGGAAAGCTGTGGTCCAATTTCTGGGGAGCATTGAGTCCTGATGGATAC
TATGCACGATCTGAAGATTATGTGGATATTGTTCAAGGGAATAGAGTAGGAGTATGGAATGTCCCATAT
ATGGCTAATGTGTACTTAATTAAAGGAAAGACACTCCGATCAGAGATGAATGAAAGGAACTATTTTGTT
CGTGATAAACTGGATCCTGATATGGCTCTTTGCCGAAATGCTAGAGAAATGGGTGTATTTATGTACATT
TCTAATAGACATGAATTTGGAAGGCTATTATCCACTGCTAATTACAATACTTCCCATTATAACAATGAC
CTCTGGCAGATTTTTGAAAATCCTGTGGACTGGAAGGAAAAGTATATAAACCGTGATTATTCAAAGATT
TTCACTGAAAATATAGTTGAACAGCCCTGTCCAGATGTCTTTTGGTTCCCCATATTTTCTGAAAAAGCC
TGTGATGAATTGGTAGAAGAAATGGAACATTACGGCAAATGGTCTGGGGGAAAACATCATGATAGCCGT
ATATCTGGTGGTTATGAAAATGTCCCAACTGATGATATCCACATGAAGCAAGTTGATCTGGAGAATGTA
TGGCTTCATTTTATCCGGGAGTTCATTGCACCAGTTACACTGAAGGTCTTTGCAGGCTATTATACGAAG
GGATTTGCACTACTGAATTTTGTAGTAAAATACTCCCCTGAACGACAGCGTTCTCTTCGTCCTCATCAT
GATGCTTCTACATTTACCATAAACATTGCACTTAATAACGTGGGAGAAGACTTTCAGGGAGGTGGTTGC
AAATTTCTAAGGTACAATTGCTCTATTGAGTCACCACGAAAAGGCTGGAGCTTCATGCATCCTGGGAGA
CTCACACATTTGCATGAAGGACTTCCTGTTAAAAATGGAACAAGATACATTGCAGTGTCATTTATAGAT
CCCTAA (SEQ ID NO: 30)
PLTP Entrez ID: 5360; OMIM: 172425; Uniprot ID: PLTP_HUMAN; ENSEMBL ID:
ENSG00000100979
ATGGCCCTCTTCGGGGCCCTCTTCCTAGCGCTGCTGGCAGGCGCACATGCAGAGTTCCCAGGCTGCAAG
ATCCGCGTCACCTCCAAGGCGCTGGAGCTGGTGAAGCAGGAGGGGCTGCGCTTTCTGGAGCAAGAGCTG
GAGACTATCACCATTCCGGACCTGCGGGGCAAAGAAGGCCACTTCTACTACAACATCTCTGAGGTGAAG
GTCACAGAGCTGCAACTGACATCTTCCGAGCTCGATTTCCAGCCACAGCAGGAGCTGATGCTTCAAATC
ACCAATGCCTCCTTGGGGCTGCGCTTCCGGAGACAGCTGCTCTACTGGTTCTTCTATGATGGGGGCTAC
ATCAACGCCTCAGCTGAGGGTGTGTCCATCCGCACTGGTCTGGAGCTCTCCCGGGATCCCGCTGGACGG
ATGAAAGTGTCCAATGTCTCCTGCCAGGCCTCTGTCTCCAGAATGCACGCGGCCTTCGGGGGAACCTTC
AAGAAGGTGTATGATTTTCTCTCCACGTTCATCACCTCAGGGATGCGCTTCCTCCTCAACCAGCAGATC
TGCCCTGTCCTCTACCACGCAGGGACGGTCCTGCTCAACTCCCTCCTGGACACCGTGCCTGTGCGCAGT
TCTGTGGACGAGCTTGTTGGCATTGACTATTCCCTCATGAAGGATCCTGTGGCTTCCACCAGCAACCTG
GACATGGACTTCCGGGGGGCCTTCTTCCCCCTGACTGAGAGGAACTGGAGCCTCCCCAACCGGGCAGTG
GAGCCCCAGCTGCAGGAGGAAGAGCGGATGGTGTATGTGGCCTTCTCTGAGTTCTTCTTCGACTCTGCC
ATGGAGAGCTACTTCCGGGCGGGGGCCCTGCAGCTGTTGCTGGTGGGGGACAAGGTGCCCCACGACCTG
GACATGCTGCTGAGGGCCACCTACTTTGGGAGCATTGTCCTGCTGAGCCCAGCAGTGATTGACTCCCCA
TTGAAGCTGGAGCTGCGGGTCCTGGCCCCACCGCGCTGCACCATCAAGCCCTCTGGCACCACCATCTCT
GTCACTGCTAGCGTCACCATTGCCCTGGTCCCACCAGACCAGCCTGAGGTCCAGCTGTCCAGCATGACT
ATGGACGCCCGTCTCAGCGCCAAGATGGCTCTCCGGGGGAAGGCCCTGCGCACGCAGCTGGACCTGCGC
AGGTTCCGAATCTATTCCAACCATTCTGCACTGGAGTCGCTGGCTCTGATCCCATTACAGGCCCCTCTG
AAGACCATGCTGCAGATTGGGGTGATGCCCATGCTCAATGAGCGGACCTGGCGTGGGGTGCAGATCCCA
CTACCTGAGGGCATCAACTTTGTGCATGAGGTGGTGACGAACCATGCGGGATTCCTCACCATCGGGGCT
GATCTCCACTTTGCCAAAGGGCTGCGAGAGGTGATTGAGAAGAACCGGCCTGCTGATGTCAGGGCGTCC
ACTGCCCCCACACCGTCCACAGCAGCTGTCTGA (SEQ ID NO: 31)
PTBP1 Entrez ID: 5725; OMIM: 600693; Uniprot ID: PTBP1_HUMAN; ENSEMBL
ID: ENSG00000011304
ATGGACGGCATTGTCCCAGATATAGCCGTTGGTACAAAGCGGGGATCTGACGAGCTTTTCTCTACTTGT
GTCACTAACGGACCGTTTATCATGAGCAGCAACTCGGCTTCTGCAGCAAACGGAAATGACAGCAAGAAG
TTCAAAGGTGACAGCCGAAGTGCAGGCGTCCCCTCTAGAGTGATCCACATCCGGAAGCTCCCCATCGAC
GTCACGGAGGGGGAAGTCATCTCCCTGGGGCTGCCCTTTGGGAAGGTCACCAACCTCCTGATGCTGAAG
GGGAAAAACCAGGCCTTCATCGAGATGAACACGGAGGAGGCTGCCAACACCATGGTGAACTACTACACC
TCGGTGACCCCTGTGCTGCGCGGCCAGCCCATCTACATCCAGTTCTCCAACCACAAGGAGCTGAAGACC
GACAGCTCTCCCAACCAGGCGCGGGCCCAGGCGGCCCTGCAGGCGGTGAACTCGGTCCAGTCGGGGAAC
CTGGCCTTGGCTGCCTCGGCGGCGGCCGTGGACGCAGGGATGGCGATGGCCGGGCAGAGCCCCGTGCTC
AGGATCATCGTGGAGAACCTCTTCTACCCTGTGACCCTGGATGTGCTGCACCAGATTTTCTCCAAGTTC
GGCACAGTGTTGAAGATCATCACCTTCACCAAGAACAACCAGTTCCAGGCCCTGCTGCAGTATGCGGAC
CCCGTGAGCGCCCAGCACGCCAAGCTGTCGCTGGACGGGCAGAACATCTACAACGCCTGCTGCACGCTG
CGCATCGACTTTTCCAAGCTCACCAGCCTCAACGTCAAGTACAACAATGACAAGAGCCGTGACTACACA
CGCCCAGACCTGCCTTCCGGGGACAGCCAGCCCTCGCTGGACCAGACCATGGCCGCGGCCTTCGGTGCA
CCTGGTATAATCTCAGCCTCTCCGTATGCAGGAGCTGGTTTCCCTCCCACCTTTGCCATTCCTCAAGCT
GCAGGCCTTTCCGTTCCGAACGTCCACGGCGCCCTGGCCCCCCTGGCCATCCCCTCGGCGGCGGCGGCA
GCTGCGGCGGCAGGTCGGATCGCCATCCCGGGCCTGGCGGGGGCAGGAAATTCTGTATTGCTGGTCAGC
AACCTCAACCCAGAGAGAGTCACACCCCAAAGCCTCTTTATTCTTTTCGGCGTCTACGGTGACGTGCAG
CGCGTGAAGATCCTGTTCAATAAGAAGGAGAACGCCCTAGTGCAGATGGCGGACGGCAACCAGGCCCAG
CTGGCCATGAGCCACCTGAACGGGCACAAGCTGCACGGGAAGCCCATCCGCATCACGCTCTCGAAGCAC
CAGAACGTGCAGCTGCCCCGCGAGGGCCAGGAGGACCAGGGCCTGACCAAGGACTACGGCAACTCACCC
CTGCACCGCTTCAAGAAGCCGGGCTCCAAGAACTTCCAGAACATATTCCCGCCCTCGGCCACGCTGCAC
CTCTCCAACATCCCGCCCTCAGTCTCCGAGGAGGATCTCAAGGTCCTGTTTTCCAGCAATGGGGGCGTC
GTCAAAGGATTCAAGTTCTTCCAGAAGGACCGCAAGATGGCACTGATCCAGATGGGCTCCGTGGAGGAG
GCGGTCCAGGCCCTCATTGACCTGCACAACCACGACCTCGGGGAGAACCACCACCTGCGGGTCTCCTTC
TCCAAGTCCACCATCTAG (SEQ ID NO: 32)
PTTG1IP Entrez ID: 754; OMIM: 603784; Uniprot ID: PTTG_HUMAN; ENSEMBL
ID: ENSG00000183255
ATGGCGCCCGGAGTGGCCCGCGGGCCGACGCCGTACTGGAGGTTGCGCCTCGGTGGCGCCGCGCTGCTC
CTGCTGCTCATCCCGGTGGCCGCCGCGCAGGAGCCTCCCGGAGCTGCTTGTTCTCAGAACACAAACAAA
ACCTGTGAAGAGTGCCTGAAGAACGTCTCCTGTCTTTGGTGCAACACTAACAAGGCTTGTCTGGACTAC
CCAGTTACAAGCGTCTTGCCACCGGCTTCCCTTTGTAAATTGAGCTCTGCACGCTGGGGAGTTTGTTGG
GTGAACTTTGAGGCGCTGATCATCACCATGTCGGTAGTCGGGGGAACCCTCCTCCTGGGCATTGCCATC
TGCTGCTGCTGCTGCTGCAGGAGGAAGAGGAGCCGGAAGCCGGACAGGAGTGAGGAGAAGGCCATGCGT
GAGCGGGAGGAGAGGCGGATACGGCAGGAGGAACGGAGAGCAGAGATGAAGACAAGACATGATGAAATC
AGAAAAAAATATGGCCTGTTTAAAGAAGAAAACCCGTATGCTAGATTTGAAAACAACTAA (SEQ ID
NO: 33)
RHBDF2 Entrez ID: 79651; OMIM:; Uniprot ID: RHDF2_HUMAN; ENSEMBL ID:
ENSG00000129667
ATGGCCTCTGCTGACAAGAATGGCGGGAGCGTGTCCTCTGTGTCCAGCAGCCGCCTGCAGAGCCGGAAG
CCACCCAACCTCTCCATCACCATCCCGCCACCCGAGAAAGAGACCCAGGCCCCTGGCGAGCAGGACAGC
ATGCTGCCTGAGAGGAAGAACCCAGCCTACTTGAAGAGCGTCAGCCTCCAGGAGCCACGCAGCCGATGG
CAGGAGAGTTCAGAGAAGCGCCCTGGCTTCCGCCGCCAGGCCTCACTGTCCCAGAGCATCCGCAAGGGC
GCAGCCCAGTGGTTTGGAGTCAGCGGCGACTGGGAGGGGCAGCGGCAGCAGTGGCAGCGCCGCAGCCTG
CACCACTGCAGCATGCGCTACGGCCGCCTGAAGGCCTCGTGCCAGCGTGACCTGGAGCTCCCCAGCCAG
GAGGCACCGTCCTTCCAGGGCACTGAGTCCCCAAAGCCCTGCAAGATGCCCAAGATTGTGGATCCGCTG
GCCCGGGGCCGGGCCTTCCGCCACCCGGAGGAGATGGACAGGCCCCACGCCCCGCACCCACCGCTGACC
CCCGGAGTCCTGTCCCTCACCTCCTTCACCAGTGTCCGTTCTGGCTACTCCCACCTGCCACGCCGCAAG
AGAATGTCTGTGGCCCACATGAGCTTGCAAGCTGCCGCTGCCCTCCTCAAGGGGCGCTCGGTGCTGGAT
GCCACCGGACAGCGGTGCCGGGTGGTCAAGCGCAGCTTTGCCTTCCCGAGCTTCCTGGAGGAGGATGTG
GTCGATGGGGCAGACACGTTTGACTCCTCCTTTTTTAGTAAGGAAGAAATGAGCTCCATGCCTGATGAT
GTCTTTGAGTCCCCCCCACTCTCTGCCAGCTACTTCCGAGGGATCCCACACTCAGCCTCCCCTGTCTCC
CCCGATGGGGTGCAAATCCCTCTGAAGGAGTATGGCCGAGCCCCAGTCCCCGGGCCCCGGCGCGGCAAG
CGCATCGCCTCCAAGGTGAAGCACTTTGCCTTTGATCGGAAGAAGCGGCACTACGGCCTCGGCGTGGTG
GGCAACTGGCTGAACCGCAGCTACCGCCGCAGCATCAGCAGCACTGTGCAGCGGCAGCTGGAGAGCTTC
GACAGCCACCGGCCCTACTTCACCTACTGGCTGACCTTCGTCCATGTCATCATCACGCTGCTGGTGATT
TGCACGTATGGCATCGCACCCGTGGGCTTTGCCCAGCACGTCACCACCCAGCTGGTGCTGCGGAACAAA
GGTGTGTACGAGAGCGTGAAGTACATCCAGCAGGAGAACTTCTGGGTTGGCCCCAGCTCGATTGACCTG
ATCCACCTGGGGGCCAAGTTCTCACCCTGCATCCGGAAGGACGGGCAGATCGAGCAGCTGGTGCTGCGC
GAGCGAGACCTGGAGCGGGACTCAGGCTGCTGTGTCCAGAATGACCACTCCGGATGCATCCAGACCCAG
CGGAAGGACTGCTCGGAGACTTTGGCCACTTTTGTCAAGTGGCAGGATGACACTGGGCCCCCCATGGAC
AAGTCTGATCTGGGCCAGAAGCGGACTTCGGGGGCTGTCTGCCACCAGGACCCCAGGACCTGCGAGGAG
CCAGCCTCCAGCGGTGCCCACATCTGGCCCGATGACATCACTAAGTGGCCGATCTGCACAGAGCAGGCC
AGGAGCAACCACACAGGCTTCCTGCACATGGACTGCGAGATCAAGGGCCGCCCCTGCTGCATCGGCACC
AAGGGCAGCTGTGAGATCACCACCCGGGAATACTGTGAGTTCATGCACGGCTATTTCCATGAGGAAGCA
ACACTCTGCTCCCAGGTGCACTGCTTGGACAAGGTGTGTGGGCTGCTGCCCTTCCTCAACCCTGAGGTC
CCAGATCAGTTCTACAGGCTCTGGCTGTCTCTCTTCCTACATGCTGGCGTGGTGCACTGCCTCGTGTCT
GTGGTCTTTCAAATGACCATCCTGAGGGACCTGGAGAAGCTGGCCGGCTGGCACCGTATCGCCATCATC
TTCATCCTCAGTGGCATCACAGGCAACCTCGCCAGTGCCATCTTTCTCCCATACCGGGCAGAGGTGGGC
CCGGCCGGCTCACAGTTCGGCCTCCTCGCCTGCCTCTTCGTGGAGCTCTTCCAGAGCTGGCCGCTGCTG
GAGAGGCCCTGGAAGGCCTTCCTCAACCTCTCGGCCATCGTGCTCTTCCTGTTCATCTGTGGCCTCCTG
CCCTGGATCGACAACATCGCCCACATCTTCGGCTTCCTCAGTGGCCTGCTGCTGGCCTTCGCCTTCCTG
CCCTACATCACCTTCGGCACCAGCGACAAGTACCGCAAGCGGGCACTCATCCTGGTGTCACTGCTGGCC
TTTGCCGGCCTCTTCGCCGCCCTCGTGCTGTGGCTGTACATCTACCCCATTAACTGGCCCTGGATCGAG
CACCTCACCTGCTTCCCCTTCACCAGCCGCTTCTGCGAGAAGTATGAGCTGGACCAGGTGCTGCACTGA
(SEQ ID NO: 34)
SERTAD1 Entrez ID: 29950; OMIM:; Uniprot ID: SRTD1_HUMAN; ENSEMBL ID:
ENSG00000197019
ATGCTGAGCAAGGGTCTGAAGCGGAAACGGGAGGAGGAGGAGGAGAAGGAACCTCTGGCAGTCGACTCC
TGGTGGCTAGATCCTGGCCACACAGCGGTGGCACAGGCACCCCCGGCCGTGGCCTCTAGCTCCCTCTTT
GACCTCTCAGTGCTCAAGCTCCACCACAGCCTGCAGCAGAGTGAGCCGGACCTGCGGCACCTGGTGCTG
GTCGTGAACACTCTGCGGCGCATCCAGGCGTCCATGGCACCCGCGGCTGCCCTGCCACCTGTGCCTAGC
CCACCTGCAGCCCCCAGTGTGGCTGACAACTTACTGGCAAGCTCGGACGCTGCCCTTTCAGCCTCCATG
GCCAGCCTCCTGGAGGACCTCAGCCACATTGAGGGCCTGAGTCAGGCTCCCCAACCCTTGGCAGACGAG
GGGCCACCAGGCCGTAGCATCGGGGGAGCAGCGCCCAGCCTGGGTGCCTTGGACCTGCTGGGCCCAGCC
ACTGGCTGTCTACTGGACGATGGGCTTGAGGGCCTGTTTGAGGATATTGACACCTCTATGTATGACAAT
GAACTTTGGGCACCAGCCTCTGAGGGCCTCAAACCAGGCCCTGAGGATGGGCCGGGCAAGGAGGAAGCT
CCGGAGCTGGACGAGGCCGAATTGGACTACCTCATGGATGTGCTGGTGGGCACACAGGCACTGGAGCGA
CCGCCGGGGCCAGGGCGCTGA (SEQ ID NO: 35)
SLC2A5 Entrez ID: 6518; OMIM: 138230; Uniprot ID: GTR5_HUMAN; ENSEMBL
ID: ENSG00000142583
ATGGAGCAACAGGATCAGAGCATGAAGGAAGGGAGGCTGACGCTTGTGCTTGCCCTGGCAACCCTGATA
GCTGCCTTTGGGTCATCCTTCCAGTATGGGTACAACGTGGCTGCTGTCAACTCCCCAGCACTGCTCATG
CAACAATTTTACAATGAGACTTACTATGGTAGGACCGGTGAATTCATGGAAGACTTCCCCTTGACGTTG
CTGTGGTCTGTAACCGTGTCCATGTTTCCATTTGGAGGGTTTATCGGATCCCTCCTGGTCGGCCCCTTG
GTGAATAAATTTGGCAGAAAAGGGGCCTTGCTGTTCAACAACATATTTTCTATCGTGCCTGCGATCTTA
ATGGGATGCAGCAGAGTCGCCACATCATTTGAGCTTATCATTATTTCCAGACTTTTGGTGGGAATATGT
GCAGGTGTATCTTCCAACGTGGTCCCCATGTACTTAGGGGAGCTGGCCCCTAAAAACCTGCGGGGGGCT
CTCGGGGTGGTGCCCCAGCTCTTCATCACTGTTGGCATCCTTGTGGCCCAGATCTTTGGTCTTCGGAAT
CTCCTTGCAAACGTAGATGGTGAGTTCAGGACATCTCGGGAGCACCCCCACCCCTTCACCACTACCCTT
GGCCCCCTCCTTGTGTTCCAAAGCCACCACCACAGGACAGGACTTTCTGCAGACTGGTCTCTTCTAACA
GGCTGGATGTCCTTGGGGGGCCCATCCTGTCCCGAGCCAACATAG (SEQ ID NO: 36)
STAMBPL1 Entrez ID: 57559; OMIM: 612352; Uniprot ID: STALP_HUMAN;
ENSEMBL ID: ENSG00000138134
ATGGATCAGCCTTTTACTGTGAATTCTCTGAAAAAGTTAGCTGCTATGCCTGACCATACAGATGTTTCC
CTAAGCCCAGAAGAGCGAGTCCGTGCCCTAAGCAAGCTTGGTTGTAATATCACCATCAGTGAAGACATC
ACTCCACGACGTTACTTTAGGTCTGGAGTAGAGATGGAGAGGATGGCGTCTGTGTATTTGGAAGAAGGA
AATTTGGAAAATGCCTTTGTTCTTTATAATAAATTTATAACCTTATTTGTAGAAAAGCTTCCTAACCAT
CGAGATTACCAGCAATGTGCAGTACCTGAAAAGCAGGATATTATGAAGAAACTGAAGGAGATTGCATTC
CCAAGGACAGATGAATTGAAAAACGACCTTTTAAAGAAATATAACGTAGAATACCAAGAATATTTGCAA
AGCAAAAACAAATATAAAGCTGAAATTCTCAAAAAATTGGAGCATCAGAGATTGATAGAGGCAGAAAGG
AAGCGGATTGCTCAGATGCGCCAGCAGCAGCTAGAATCGGAGCAGTTTCTGTTTTTCGAAGATCAACTC
AAGAAGCAAGAGTTAGCCCGAGGTCAAATGCGAAGTCAGCAAACCTCAGGGCTGTCAGAGCAGATTGAT
GGGAGCGCTTTGTCCTGCTTTTCCACACACCAGAACAATTCCTTGCTGAATGTATTTGCAGATCAACCT
AATAAAAGTGATGCAACCAATTATGCTAGCCACTCTCCTCCTGTAAACAGGGCCTTAACACCAGCTGCT
ACTCTAAGTGCTGTTCAGAATTTAGTGGTTGAAGGACTGCGATGTGTAGTTTTGCCAGAAGATCTTTGC
CACAAATTTCTGCAACTGGCAGAATCTAATACAGTGAGAGGAATAGAAACCTGTGGAATACTCTGTGGA
AAACTGACACATAATGAATTTACTATTACCCATGTAATTGTGCCAAAGCAGTCTGCGGGACCAGACTAT
TGTGACATGGAGAATGTAGAGGAATTATTCAATGTTCAGGATCAACATGATCTCCTCACTCTAGGATGG
ATCCATACACATCCCACTCAAACTGCATTTTTATCCAGCGTTGATCTTCACACTCACTGTTCCTATCAA
CTCATGTTGCCAGAGGCCATTGCCATTGTTTGCTCACCAAAGCATAAAGACACTGGCATCTTCAGGCTC
ACCAATGCTGGCATGCTTGAGGTTTCTGCTTGTAAAAAAAAGGGCTTTCATCCACACACCAAGGAGCCC
AGGCTGTTCAGTATATGCAAACATGTGTTGGTAAAAGACATAAAAATAATTGTGTTGGATCTGAGGTGA
(SEQ ID NO: 37)
TESC Entrez ID: 54997; OMIM: 611585; Uniprot ID: TESC_HUMAN; ENSEMBL
ID: ENSG00000088992
ATGGGCGCTGCCCACTCCGCGTCTGAGGAGGTGCGGGAGCTCGAGGGCAAGACCGGCTTCTCATCGGAT
CAGATCGAGCAGCTCCATCGGAGATTTAAGCAGCTGAGTGGAGATCAGCCTACCATTCGGAACCTGCGC
AAGGGACCCAGTGGCCTGGCTGATGAGATCAATTTCGAGGACTTCCTGACCATCATGTCCTACTTCCGG
CCCATCGACACCACCATGGACGAGGAACAGGTGGAGCTGTCCCGGAAGGAGAAGCTGAGATTTCTGTTC
CACATGTACGACTCGGACAGCGACGGCCGCATCACTCTGGAAGAATATCGAAATGTGGTCGAGGAGCTG
CTGTCGGGAAACCCTCACATCGAGAAGGAGTCCGCTCGCTCCATCGCCGACGGGGCCATGATGGAGGCG
GCCAGCGTGTGCATGGGGCAGATGGAGCCTGATCAGGTGTACGAGGGGATCACCTTCGAGGACTTCCTG
AAGATCTGGCAGGGGATCGACATTGAGACCAAGATGCACGTCCGCTTCCTTAACATGGAAACCATGGCC
CTCTGCCACTGA (SEQ ID NO: 38)
TIMP1 Entrez ID: 7076; OMIM: 305370; Uniprot ID: TIMP1_HUMAN; ENSEMBL
ID: ENSG00000102265
ATGGCCCCCTTTGAGCCCCTGGCTTCTGGCATCCTGTTGTTGCTGTGGCTGATAGCCCCCAGCAGGGCC
TGCACCTGTGTCCCACCCCACCCACAGACGGCCTTCTGCAATTCCGACCTCGTCATCAGGGCCAAGTTC
GTGGGGACACCAGAAGTCAACCAGACCACCTTATACCAGCGTTATGAGATCAAGATGACCAAGATGTAT
AAAGGGTTCCAAGCCTTAGGGGATGCCGCTGACATCCGGTTCGTCTACACCCCCGCCATGGAGAGTGTC
TGCGGATACTTCCACAGGTCCCACAACCGCAGCGAGGAGTTTCTCATTGCTGGAAAACTGCAGGATGGA
CTCTTGCACATCACTACCTGCAGTTTTGTGGCTCCCTGGAACAGCCTGAGCTTAGCTCAGCGCCGGGGC
TTCACCAAGACCTACACTGTTGGCTGTGAGGAATGCACAGTGTTTCCCTGTTTATCCATCCCCTGCAAA
CTGCAGAGTGGCACTCATTGCTTGTGGACGGACCAGCTCCTCCAAGGCTCTGAAAAGGGCTTCCAGTCC
CGTCACCTTGCCTGCCTGCCTCGGGAGCCAGGGCTGTGCACCTGGCAGTCCCTGCGGTCCCAGATAGCC
TGA (SEQ ID NO: 39)
TNPO1 Entrez ID: 3842; OMIM: 602901; Uniprot ID: TNPO1_HUMAN; ENSEMBL
ID: ENSG00000083312
ATGGTGTGGGACCGGCAAACCAAGATGGAGTATGAGTGGAAACCTGACGAGCAAGGGCTTCAGCAAATC
CTGCAGCTGTTGAAGGAGTCCCAGTCCCCAGACACCACCATCCAGAGAACCGTGCAACAAAAACTGGAA
CAACTTAATCAGTATCCAGACTTTAACAACTACTTGATTTTTGTTCTTACAAAATTAAAATCTGAAGAT
GAACCCACAAGATCATTGAGTGGTCTTATCTTGAAGAATAATGTGAAAGCACACTTTCAGAACTTCCCA
AATGGTGTAACAGACTTTATTAAAAGTGAATGTTTAAATAATATTGGTGACTCCTCTCCTCTGATTAGA
GCCACTGTTGGTATTTTGATCACAACTATAGCCTCCAAGGGAGAATTGCAGAATTGGCCTGACCTCTTA
CCAAAACTCTGTAGCCTGTTGGATTCTGAAGATTATAATACCTGTGAGGGAGCATTTGGTGCCCTTCAG
AAGATTTGTGAAGATTCTGCTGAGATTTTAGACAGTGATGTTTTAGATCGTCCTCTCAACATCATGATT
CCCAAATTTTTACAGTTCTTCAAGCATAGTAGTCCAAAAATAAGGTCTCACGCTGTTGCATGTGTCAAT
CAGTTTATCATCAGTAGGACTCAAGCTCTAATGTTGCACATTGATTCTTTTATTGAGAATCTCTTTGCA
TTAGCTGGTGATGAAGAACCAGAGGTACGGAAAAATGTGTGCCGAGCACTTGTGATGTTGCTCGAAGTT
CGAATGGATCGCCTGCTTCCTCACATGCATAATATAGTTGAGTACATGCTACAGAGGACTCAAGATCAA
GATGAAAATGTGGCTTTAGAAGCCTGTGAATTTTGGCTAACTTTAGCTGAACAGCCAATATGCAAAGAT
GTACTCGTAAGGCATCTTCCTAAGTTGATTCCTGTGTTAGTGAATGGCATGAAGTACTCAGACATAGAT
ATTATCCTACTTAAGGGTGATGTTGAAGAAGACGAAACGATTCCTGATAGTGAACAGGATATACGGCCA
CGTTTTCACCGATCGAGGACGGTGGCTCAGCAGCATGATGAAGATGGAATTGAAGAGGAAGATGATGAT
GATGATGAAATTGATGATGATGATACAATTTCTGACTGGAATCTAAGAAAATGTTCTGCTGCTGCCCTG
GATGTTCTTGCAAATGTGTATCGTGATGAACTGCTGCCACATATTTTGCCCCTTTTGAAAGAATTACTT
TTTCATCATGAATGGGTTGTTAAAGAATCAGGCATTTTGGTTTTAGGAGCAATTGCTGAAGGTTGCATG
CAGGGCATGATTCCATACTTGCCTGAGCTTATTCCTCACCTTATTCAGTGCCTCTCTGATAAAAAGGCT
CTTGTGCGTTCCATAACATGCTGGACTCTTAGCCGCTATGCACACTGGGTGGTCAGCCAGCCGCCAGAC
ACGTACCTGAAGCCATTAATGACAGAATTGCTAAAGCGCATCCTGGACAGCAACAAGAGAGTACAAGAA
GCTGCCTGCAGTGCCTTTGCTACCCTAGAAGAGGAGGCTTGTACAGAACTTGTTCCTTACCTTGCTTAT
ATACTTGATACCCTGGTCTTTGCATTTAGTAAATACCAGCATAAGAACCTGCTCATTCTTTACGATGCC
ATAGGAACATTAGCAGATTCAGTAGGACATCATTTAAACAAACCAGAATATATTCAGATGCTAATGCCT
CCACTGATCCAGAAATGGAACATGTTAAAGGATGAAGATAAAGATCTCTTCCCTTTACTTGAGTGCCTA
TCTTCAGTTGCCACAGCACTGCAGTCTGGATTCCTTCCGTACTGTGAACCTGTGTATCAGCGTTGTGTA
AACCTAGTACAGAAGACTCTTGCACAAGCCATGCTAAACAATGCTCAACCAGATCAATATGAAGCTCCA
GATAAAGATTTTATGATAGTGGCTCTTGATTTACTGAGTGGCCTGGCTGAAGGACTTGGAGGCAACATT
GAACAGCTGGTAGCCCGAAGTAACATCCTGACACTAATGTATCAGTGCATGCAGGATAAAATGCCAGAA
GTTCGACAGAGTTCTTTTGCCCTGTTAGGTGACCTCACAAAAGCTTGCTTTCAGCATGTTAAGCCTTGT
ATAGCTGATTTCATGCCAATATTGGGAACCAACCTAAATCCAGAATTCATTTCAGTCTGCAACAATGCC
ACATGGGCAATTGGAGAAATCTCCATTCAAATGGGTATAGAGATGCAGCCTTATATTCCTATGGTGTTG
CACCAGCTTGTAGAAATCATTAACAGACCCAACACACCAAAGACGTTGTTAGAGAATACAGCAATAACA
ATTGGTCGTCTTGGTTACGTTTGTCCTCAAGAGGTGGCCCCCATGCTACAGCAGTTTATAAGACCCTGG
TGCACCTCTCTGAGAAACATAAGAGACAATGAGGAAAAGGATTCAGCATTCCGTGGAATTTGTACCATG
ATCAGTGTGAATCCCAGTGGCGTAATCCAAGATTTTATATTTTTTTGTGATGCCGTTGCATCATGGATT
AACCCAAAAGATGATCTCAGAGACATGTTCTGTAAGATCCTTCATGGATTTAAAAATCAAGTTGGCGAT
GAAAATTGGAGGCGTTTCTCTGACCAGTTTCCTCTTCCCTTAAAAGAGCGTCTTGCAGCTTTTTATGGT
GTTTAA (SEQ ID NO: 40)
TXNIP Entrez ID: 10628; OMIM: 606599; Uniprot ID: TXNIP_HUMAN; ENSEMBL
ID: ENSG00000117289
ATGGTGATGTTCAAGAAGATCAAGTCTTTTGAGGTGGTCTTTAACGACCCTGAAAAGGTGTACGGCAGT
GGCGAGAAGGTGGCTGGCCGGGTGATAGTGGAGGTGTGTGAAGTTACTCGTGTCAAAGCCGTTAGGATC
CTGGCTTGCGGAGTGGCTAAAGTGCTTTGGATGCAGGGATCCCAGCAGTGCAAACAGACTTCGGAGTAC
CTGCGCTATGAAGACACGCTTCTTCTGGAAGACCAGCCAACAGGTGAGAATGAGATGGTGATCATGAGA
CCTGGAAACAAATATGAGTACAAGTTCGGCTTTGAGCTTCCTCAGGGGCCTCTGGGAACATCCTTCAAA
GGAAAATATGGGTGTGTAGACTACTGGGTGAAGGCTTTTCTTGACCGCCCGAGCCAGCCAACTCAAGAG
ACAAAGAAAAACTTTGAAGTAGTGGATCTGGTGGATGTCAATACCCCTGATTTAATGGCACCTGTGTCT
GCTAAAAAAGAAAAGAAAGTTTCCTGCATGTTCATTCCTGATGGGCGGGTGTCTGTCTCTGCTCGAATT
GACAGAAAAGGATTCTGTGAAGGTGATGAGATTTCCATCCATGCTGACTTTGAGAATACATGTTCCCGA
ATTGTGGTCCCCAAAGCTGCCATTGTGGCCCGCCACACTTACCTTGCCAATGGCCAGACCAAGGTGCTG
ACTCAGAAGTTGTCATCAGTCAGAGGCAATCATATTATCTCAGGGACATGCGCATCATGGCGTGGCAAG
AGCCTTCGGGTTCAGAAGATCAGGCCTTCTATCCTGGGCTGCAACATCCTTCGAGTTGAATATTCCTTA
CTGATCTATGTTAGCGTTCCTGGATCCAAGAAGGTCATCCTTGACCTGCCCCTGGTAATTGGCAGCAGA
TCAGGTCTAAGCAGCAGAACATCCAGCATGGCCAGCCGAACCAGCTCTGAGATGAGTTGGGTAGATCTG
AACATCCCTGATACCCCAGAAGCTCCTCCCTGCTATATGGATGTCATTCCTGAAGATCACCGATTGGAG
AGCCCAACCACTCCTCTGCTAGATGACATGGATGGCTCTCAAGACAGCCCTATCTTTATGTATGCCCCT
GAGTTCAAGTTCATGCCACCACCGACTTATACTGAGGTGGATCCCTGCATCCTCAACAACAATGTGCAG
TGA (SEQ ID NO: 41)
VAMP1 Entrez ID: 6843; OMIM: 185880; Uniprot ID: VAMP1_HUMAN; ENSEMBL
ID: ENSG00000139190
ATGTCTGCTCCAGCTCAGCCACCTGCTGAAGGGACAGAAGGGACTGCCCCAGGTGGGGGTCCCCCTGGC
CCTCCTCCTAACATGACCAGTAACAGACGACTACAGCAAACCCAGGCACAAGTGGAGGAGGTGGTGGAC
ATCATACGTGTGAACGTGGACAAGGTCCTGGAGAGGGACCAGAAGCTGTCAGAGCTGGATGACCGAGCT
GATGCCTTGCAGGCAGGAGCATCACAATTTGAGAGCAGTGCTGCCAAGCTAAAGAGGAAGTATTGGTGG
AAAAACTGCAAGATGATGATCATGCTGGGAGCCATCTGTGCCATCATCGTGGTAGTTATTGTAATCTAC
TTTTTTACTTGA (SEQ ID NO: 42)
VHL Entrez ID: 7428; OMIM: 608537; Uniprot ID: VHL_HUMAN; ENSEMBL ID:
ENSG00000134086
ATGCCCCGGAGGGCGGAGAACTGGGACGAGGCCGAGGTAGGCGCGGAGGAGGCAGGCGTCGAAGAGTAC
GGCCCTGAAGAAGACGGCGGGGAGGAGTCGGGCGCCGAGGAGTCCGGCCCGGAAGAGTCCGGCCCGGAG
GAACTGGGCGCCGAGGAGGAGATGGAGGCCGGGCGGCCGCGGCCCGTGCTGCGCTCGGTGAACTCGCGC
GAGCCCTCCCAGGTCATCTTCTGCAATCGCAGTCCGCGCGTCGTGCTGCCCGTATGGCTCAACTTCGAC
GGCGAGCCGCAGCCCTACCCAACGCTGCCGCCTGGCACGGGCCGCCGCATCCACAGCTACCGAGGTCAC
CTTTGGCTCTTCAGAGATGCAGGGACACACGATGGGCTTCTGGTTAACCAAACTGAATTATTTGTGCCA
TCTCTCAATGTTGACGGACAGCCTATTTTTGCCAATATCACACTGCCAGTGTATACTCTGAAAGAGCGA
TGCCTCCAGGTTGTCCGGAGCCTAGTCAAGCCTGAGAATTACAGGAGACTGGACATCGTCAGGTCGCTC
TACGAAGATCTGGAAGACCACCCAAATGTGCAGAAAGACCTGGAGCGGCTGACACAGGAGCGCATTGCA
CATCAACGGATGGGAGATTGA (SEQ ID NO: 43)
ZFP36L1 Entrez ID: 677; OMIM: 601064; Uniprot ID: TISB_HUMAN; ENSEMBL
ID: ENSG00000185650
ATGACCACCACCCTCGTGTCTGCCACCATCTTCGACTTGAGCGAAGTTTTATGCAAGGGTAACAAGATG
CTCAACTATAGTGCTCCCAGTGCAGGGGGTTGCCTGCTGGACAGAAAGGCAGTGGGCACCCCTGCTGGT
GGGGGCTTCCCTCGGAGGCACTCAGTCACCCTGCCCAGCTCCAAGTTCCACCAGAACCAGCTCCTCAGC
AGCCTCAAGGGTGAGCCAGCCCCCGCTCTGAGCTCGCGAGACAGCCGCTTCCGAGACCGCTCCTTCTCG
GAAGGGGGCGAGCGGCTGCTGCCCACCCAGAAGCAGCCCGGGGGCGGCCAGGTCAACTCCAGCCGCTAC
AAGACGGAGCTGTGCCGCCCCTTTGAGGAAAACGGTGCCTGTAAGTACGGGGACAAGTGCCAGTTCGCA
CACGGCATCCACGAGCTCCGCAGCCTGACCCGCCACCCCAAGTACAAGACGGAGCTGTGCCGCACCTTC
CACACCATCGGCTTTTGCCCCTACGGGCCCCGCTGCCACTTCATCCACAACGCTGAAGAGCGCCGTGCC
CTGGCCGGGGCCCGGGACCTCTCCGCTGACCGTCCCCGCCTCCAGCATAGCTTTAGCTTTGCTGGGTTT
CCCAGTGCCGCTGCCACCGCCGCTGCCACCGGGCTGCTGGACAGCCCCACGTCCATCACCCCACCCCCT
ATTCTGAGCGCCGATGACCTCCTGGGCTCACCTACCCTGCCCGATGGCACCAATAACCCTTTTGCCTTC
TCCAGCCAGGAGCTGGCAAGCCTCTTTGCCCCTAGCATGGGGCTGCCCGGGGGTGGCTCCCCGACCACC
TTCCTCTTCCGGCCCATGTCCGAGTCCCCTCACATGTTTGACTCTCCCCCCAGCCCTCAGGATTCTCTC
TCGGACCAGGAGGGCTACCTGAGCAGCTCCAGCAGCAGCCACAGTGGCTCAGACTCCCCGACCTTGGAC
AACTCAAGACGCCTGCCCATCTTCAGCAGACTTTCCATCTCAGATGACTAA (SEQ ID NO: 44)
As is known in the art, individuals diagnosed with autism shown dysregulated gene expression in leukocytes (see, e.g. Nishimura et al., Human Molecular Genetics 2007 16 (14): 1682-1698). Moreover, gene ontology enrichment analysis showed that genes upregulated in autistic cortex were enriched for gene ontology categories implicated in immune and inflammatory response. Genes including those identified in Table B were shown to have overlapping expression patterns with brain and blood cells in one or more data sets, data supporting their utility as peripheral biomarkers.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. A method of identifying a test mammalian cell having a gene expression profile observed in individuals diagnosed with autism comprising:
observing an expression profile of at least one gene comprising a sequence selected from the group consisting of SEQ ID NOs: 1-44 in the test mammalian cell;
wherein an expression profile of a gene in the group that is at least two standard deviations from a mean expression profile of the gene in a control mammalian cell obtained from an individual not affected with autism identifies the test mammalian cell as having a gene expression profile observed in individuals diagnosed with autism.

2. The method of claim 1, wherein the expression of a gene in the group is at least three, four or five standard deviations from the mean expression of the gene observed in a control mammalian cell.

3. The method of claim 1, wherein the expression level of a gene in the group is at least 20, 30, 40, 50, 60 or 70% above or below the expression level of the gene observed in the control mammalian cell.

4. The method of claim 1, wherein mRNA expression is observed.

5. The method of claim 4, wherein the expression profile is observed using:
(a) a microarray of polynucleotides; and/or
(b) a quantitative PCR (qPCR) process.

6. The method of claim 1, wherein an expression profile of at least, 2, 3, 4, 5, 6, 7, 8, 9 or 10 genes in the group is observed.

7. The method of claim 1, wherein the expression profile of the test mammalian cell is observed using a computer system comprising a processor element and a memory storage element adapted to process and store data from one or more expression profiles.

8. The method of claim 1, wherein the test mammalian cell or the control mammalian cell is a leukocyte obtained from the peripheral blood.

9. The method of claim 1, wherein the test mammalian cell is obtained from an individual previously identified as exhibiting restricted repetitive behaviors or speech delay.

10. The method of claim 1, wherein the control mammalian cell is obtained from an individual previously identified as not exhibiting restricted repetitive behaviors or speech delay.

11. The method of claim 1, wherein the test mammalian cell and the control mammalian cell are obtained from individuals who are related as siblings or as a parent and a child.

12. The method of claim 1, wherein the test mammalian cell is obtained from an individual identified as having a family member previously identified as exhibiting restricted repetitive behaviors or speech delay.

13. The method of claim 1, wherein the test mammalian cell is obtained from an individual where at least one evaluation is performed from a diagnostic procedure for autism comprising:
(a) a Autism Diagnostic Interview (ADI-R);
(b) an Autism Diagnostic Observation Schedule (ADOS);
(c) an IQ surrogate test based on Raven's Progressive Matrices; or
(d) observations of restricted repetitive behaviors or speech delay.

14. The method of claim 13, wherein the at least one evaluation is performed prior to observing the expression profile of the at least one gene.

15. A kit comprising:
a container;
a primer composition contained within said container, wherein the primer composition includes a polymerase chain reaction (PCR) primer effective in the quantitative real time analysis of the mRNA expression levels of one or more genes selected from the group consisting of SEQ ID NOs: 1-44; and
a buffer composition.

16. The kit of claim 15, wherein the kit comprises a plurality of chain reaction (PCR) primers effective in the quantitative real time analysis of the mRNA expression levels of different genes selected from the group consisting of SEQ ID NOs: 1-44.

17. The kit of claim 15, further comprising a computer readable a memory storage element adapted to process and store data from one or more expression profiles.

18. The kit of claim 17, wherein the memory storage element organizes expression profile data into a format adapted for electronic comparisons with a library of expression profile data.

19. The kit of claim 1, wherein the primer is adapted to bind to mRNA expressed by a human leukocyte.

20. A method of observing an effect of a compound on an expression profile of at least one gene comprising a sequence selected from the group consisting of SEQ ID NOs: 1-44, the method comprising the steps of:
observing an expression profile of the at least one gene in the presence of the compound; and
comparing the expression profile that is observed in the presence of the compound with the expression profile that is observed in the absence of the compound;
so that the effect of the compound on an expression profile of the at least one gene is observed.
